(19) United States
(12) Patent Application Publication
(10) Pub. No.: US 2006/0058553 A1
(43) Pub. Date: Mar. 16, 2006

(54) Novel bicyclic hydroxamates as inhibitors of histone deacetylase

(75) Inventors: Ellen M Leahy, San Leandro, CA (US); Erik J Verner, Foster City, CA (US)

Correspondence Address: CELERA, AN APPLERA CORPORATION BUSINESS, 180 KIMBALL WAY, SOUTH SAN FRANCISCO, CA, 94080, US

(73) Assignee: AXYS PHARMACEUTICALS, INC., SO. San Francisco, CA (US)

(21) Appl. No.: 10/503,508

(22) PCT Filed: Feb. 7, 2003
(86) PCT No.: PCT/US03/03846

§ 371 Date: Oct. 12, 2005

Related U.S. Application Data
(60) Provisional application No. 60/355,700, filed on Feb. 7, 2002.

(51) Int. Cl. C07D 235/04

(52) U.S. Cl. 562/622; 564/123; 548/305.4

(57) ABSTRACT

The present invention is directed to certain bicyclic hydroxamate derivatives that are inhibitors of histone deacetylase and are therefore useful in the treatment of diseases associated with histone deacetylase activity. Pharmaceutical compositions and processes for preparing these compounds are also disclosed

### BACKGROUND OF THE INVENTION

1. Field of the Invention

The present invention is directed to certain bicyclic hydroxamate derivatives that are inhibitors of histone deacetylase and are therefore useful in the treatment of diseases associated with histone deacetylase activity. Pharmaceutical compositions and processes for preparing these compounds are also disclosed.

2. State of the Art

Interest in histone deacetylase enzymes (HDACs) as targets for pharmaceutical development has centered on the role of HDACs in regulating genes associated with cell-cycle progression and the development and progression of cancer (reviewed in Kramer et. al. 2001. Trends Endocrinol. Metab. 12:294-300). Several studies have shown that treatment of various cell lines with HDAC inhibitors leads to hyper acetylation of histone proteins and cell-cycle arrest in late G₁ phase or at the G₂/M transition. Genes involved in the cell cycle that have been shown to be up regulated by HDAC inhibitors include p21, p27, p53 and cyclin E. Cyclin A and cyclin D have been reported to be down regulated by HDAC inhibitors. In tumor cell lines, several studies have shown that treatment with HDAC inhibitors can lead to growth inhibition, growth arrest, terminal differentiation and/or apoptosis. In vivo studies have demonstrated growth inhibition of tumors and a reduction in tumor metastasis as a result of treatment with HDAC inhibitors.

The clearest link between abnormal HDAC activity and cancer occurs in acute promyelocytic leukemia. In this condition, a chromosomal translocation leads to the fusion of the retinoic acid receptor RARα with the promyelocytic leukemia (PML) or promyelocytic leukemia zinc-finger (PLZF) proteins. Both PML-RARα and PLZF-RARα promote the progression of leukemia by repressing retinoic acid-regulated genes through the abnormal recruitment of SMRT-mSin3-HDAC complex (Lin et. al., 1998, Nature 391:811-814; Grignani et al., 1998, Nature 391:815-818). Whereas the PML-RARα form of the disease is treatable with retinoic acid, the PLZF-RARα form is resistant to this treatment. For a patient with the retinoic acid-resistant form of the disease, the addition of the HDAC inhibitor sodium butyrate to the dosing regimen led to complete clinical and cytogenic remission (Warrell et al., 1998, J. Natl. Cancer. Inst. 90:1621-1625). HDACs have also been associated with Huntington's disease (Steffan, et al., Nature 413:739-744, “Histone deacetylase inhibitors arrest polyglutamine-dependent neurodegeneration in Drosophila”).

In summary, an increase in HDAC activity contributes to the pathology and/or symptomatology of a number of diseases. Accordingly, molecules that inhibit the activity of HDAC are useful as therapeutic agents in the treatment of such diseases.

### SUMMARY OF THE INVENTION

In a first aspect, this invention provides a compound of Formula I: 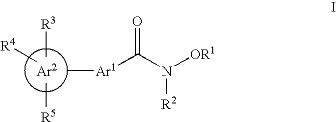
wherein:
R¹ is hydrogen or alkyl;
R² is hydrogen;
Ar¹ is phenylene or a six membered heteroarylene ring containing one or two nitrogen ring atoms, the rest of the ring atoms being carbon; wherein said Ar¹ group is optionally substituted with one or two groups independently selected from alkyl, halo, hydroxy, alkoxy, haloalkoxy, or haloalkyl;
Ar² is aryl, benzimidazol-2-yl, cycloalkyl or heterocycloalkyl;
R³ is hydrogen, alkyl, halo, hydroxy, or alkoxy; and
R⁴ and R⁵ are independently selected from the group consisting of hydrogen, alkyl, halo, haloalkyl, nitro, cyano, carboxy, carboxyalkyl, alkoxycarbonyl, optionally substituted phenyl, optionally substituted heteroaryl, optionally substituted heterocycloalkyl, cycloalkyl, heterocycloaminoalkyl, —X—R⁶, or —(C₁₋₆alkylene)-Y—R⁷ where X and Y are independently —O—, —S—, —SO—, —SO₂—, —NR⁸—, —CO—, —NR⁹CO—, —CONR¹⁰—, —NR¹¹SO₂—, —SO₂NR¹²—, —NHC(O)O—, —OC(O)NH—, —NR¹³CONR¹⁴—, or —NR¹⁵SO₂NR¹⁶— where R⁶ and R⁷ are independently hydrogen, alkyl, hydroxyalkyl, optionally substituted phenyl, optionally substituted heteroaryl, optionally substituted heterocycloalkyl, cycloalkyl, optionally substituted phenylalkyl, optionally substituted phenoxyalkyl, optionally substituted phenylalkenyl, optionally substituted phenylaminoalkyl, optionally substituted heteroaralkyl, optionally substituted heteroaryloxyalkyl, optionally substituted heterocycloalkylalkyl, or cycloalkylalkyl, R⁸, R⁹, R¹¹, R¹³, and R¹⁵ are independently hydrogen, alkyl, hydroxyalkyl, alkoxyalkyl, or optionally substituted phenylalkyl; R¹⁰, R¹², R¹⁴, and R¹⁶ are independently hydrogen, alky, optionally substituted phenylalkyl, alkoxy, hydroxyalkyl, haloalkyl, alkoxyalkyl, carboxyalkyl, cyanoalkyl, aminoalkyl, aminocarbonylalkyl, alkylaminocarbonylalkyl, dialkylaminocarbonylalkyl, or acyl or R⁴ and R⁵ together form methylenedioxy; and individual isomers, mixtures of isomers; or a pharmaceutically acceptable salt thereof provided that: (i) at least one of R³, R⁴ and R⁵ is not hydrogen; (ii) when Ar² is cycloalkyl, then at least two of R³, R⁴ and R⁵ are hydrogen; (iii) when R¹ and R³ are hydrogen, Ar¹ is phenylene and Ar² is phenyl, and one of R⁴ and R⁵ is methoxy, then the other of R⁴ and R⁵ is not —OR⁶ where R⁶ is cyclopentyl or phenylpentyl; (iv) when Ar¹ is phenylene and Ar² is phenyl then at least one of R³, R⁴ and R⁵ is not alkyl; (v) when Ar¹ is phenylene, Ar² is aryl and is located at the 3-position of the phenylene ring, then Ar² is not substituted with an optionally substituted phenyl; (vi) when Ar¹ is phenylene and Ar² is phenyl, and R⁴ or R⁵ is —CONR¹⁰R⁶ or C₁₋₆alkylene)-CONR¹⁰R⁷ then said R⁴ or R⁵ is not located at the 4-position of the phenyl ring; and (vii) when Ar¹ is phenylene and Ar² is phenyl and two of R³, R⁴ and R⁵ are hydrogen, then the remaining of R³, R⁴ and R⁵ is not nitro.
   A. Preferably, the compound of Formula I is represented by Formula Ia: 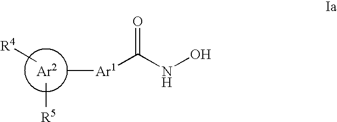
   wherein:
Ar¹ is phenylene or a six membered heteroarylene ring containing one or two nitrogen ring atoms, the rest of the ring atoms being carbon; wherein said Ar¹ group is optionally substituted with one or two groups independently selected from alkyl, halo, hydroxy, alkoxy, trifluoromethoxy, or trifluoromethyl;
Ar² is aryl, benzimidazol-2-yl, cycloalkyl or heterocycloalkyl and is located at the 4-position of Ar¹;
R⁴ and R⁵ are independently selected from the group consisting of hydrogen, alkyl, halo, haloalkyl, cyano, carboxy, carboxyalkyl, alkoxycarbonyl, optionally substituted phenyl, optionally substituted heteroaryl, optionally substituted heterocycloalkyl, cycloalkyl, —X—R⁶, or —(C₁₋₆alkylene)-Y—R⁷ where X and Y are independently —O—, —S—, —SO—, —SO₂—, —NR⁸—, —CO, —NR⁹CO—, —CONR¹⁰—, —NR¹¹SO₂—, —SO₂NR¹²—, —NHC(O)O—, —OC(O)NH—, —NR¹³CONR¹⁴—, or —NR¹⁵SO₂NR¹⁶— where R⁶ and R⁷ are independently hydrogen, alkyl, hydroxyalkyl, optionally substituted phenyl, optionally substituted heteroaryl, optionally substituted heterocycloalkyl, cycloalkyl, optionally substituted phenylalkyl, optionally substituted phenoxyalkyl, optionally substituted heteroaralkyl, optionally substituted heteroaryloxyalkyl, optionally substituted heterocycloalkylalkyl, or cycloalkylalkyl, R⁸, R⁹, R¹¹, R¹³, and R¹⁵ are independently hydrogen, alkyl, or optionally substituted phenylalkyl; R¹⁰, R¹², R¹⁴, and R¹⁶ are independently hydrogen, alkyl, optionally substituted phenylalkyl, alkoxy, hydroxyalkyl, haloalkyl, alkoxyalkyl, carboxyalkyl, cyanoalkyl, aminoalkyl, aminocarbonylalkyl, alkylaminocarbonylalkyl, dialkylaminocarbonylalkyl, or acyl or R⁴ and R⁵ together form methylenedioxy; and individual isomers, mixtures of isomers; or a pharmaceutically acceptable salt thereof.

Within this preferred group (A), a more preferred group of compounds is that wherein:
(i) Ar¹ is phenylene, Ar² is phenyl wherein R⁴ is hydrogen and R⁵ is cyano, optionally substituted phenyl, optionally substituted heteroaryl, —X—R⁶ (where X is —O—, —NH—, —SO₂—, —CO—, —NHCO—, —CONR¹⁰—, —NHSO₂— or —NHCONH— [where R¹⁰ is hydrogen, alkyl or haloalkyl; and R⁶ is alkyl (except when X is —O— or —NH—), hydroxyalkyl, optionally substituted phenyl, optionally substituted heteroaryl, optionally substituted heterocycloalkyl, optionally substituted phenylalkyl, optionally substituted phenoxyalkyl, or optionally substituted heteroaralkyl], or —(C₁₋₆alkylene)-Y—R⁷ [where Y is —CO— or —CONR¹⁰—; and R⁷ is alkyl, hydroxyalkyl, optionally substituted phenyl, optionally substituted heteroaryl, optionally substituted heterocycloalkyl, optionally substituted phenylalkyl, optionally substituted phenoxyalkyl, or optionally substituted heteroaralkyl and R¹⁰ is hydrogen or alkyl] and is located at the the 3-postion of the phenyl ring. Even more preferably R⁵ is —NHSO₂CH₃, —SO₂CH₃, thiophen-3-yl, cyano, —NHSO₂phenyl, hydroxymethyl, —NHSO₂(3-chlorophenyl), —NHSO₂(4-fluorophenyl), —NHSO₂(3,4-dichlorophenyl), —NHCOphenyl, —NHSO₂(benzyl), —NHSO₂(4-chlorophenyl), —NHSO₂(3-trifluoromethylphenyl), —NHSO₂(4-methoxyphenyl), —NHCO(3,4-dichlorophenyl), —NHCONH(3-methoxyphenyl), —NHCO(3,4-dimethoxyphenyl), —NHSO₂(2,5-dimethoxyphenyl), —NHSO₂(4-trifluoromethoxyphenyl), —NHCO(3-fluorophenyl), —NHCO(2,4-dichlorophenyl), —CONHphenyl, —NHCO(4-methylphenyl), —NHCO(3-trifluoromethylphenyl), -Obenzyl, —O-(3-methoxybenzyl), —NHCO(3,4-methylenedioxyphenyl), —NHCO(4-methoxyphenyl), —CONH(benzyl), —CONH(3,3,3-trifluoroethyl), —NHCO(3-methylbutyl), —CONH(CH₃), —NHCOCH(CH₃)₂, —NHCO(benzyl), —NHCO(CH₂)₂phenyl, —NHCO(CH₂)₂(4-trifluorophenyl), —NHCO(CH₂)₂(4-methoxyphenyl), —NHCO(3,5-dichlorophenyl), —NHCO(CH₂O)phenyl, —NHCO(3-methylbutyl), —NHCOCH₂thiophen-2-yl, —NHCO(CH₂)₂(2,4-dichlorophenyl), —NHCO(CH₂)₂(3,4-methylenedioxyphenyl), —NHCO(4-trifluoromethylphenyl), —NHCO(4-ethoxyphenyl), —NHCO(4-dimethylaminophenyl), —NHCO(4-fluorophenyl), —NHCO(2,4-difluorophenyl), —NHCO(4-chlorophenyl), —CON(CH₃)₂, —NHCO(4-isopropylphenyl), —NHCO(4-trifluoromethoxyphenyl), —NHCO(3-fluoro-4-methoxyphenyl), —NHCO(4-methoxy-2-methylphenyl), —NHCO(2,4-dimethoxyphenyl), —NHCO(4-chloro-2-methoxyphenyl), —NHCO(pyridin-4-yl), —NHCO(pyridin-3-yl), —COmorpholin-4-yl, —CON(CH₃)(phenyl), —CONH(4-chlorophenyl), —CONH(CH₂)-2(4-methoxyphenyl), —CONH(4-chlorobenzyl), —CONH(CH₂)₂phenyl, —CONH(CH₂)₂OH, —COpiperidin-1-yl, —NHCO(2-methylphenyl), —NHCO(2,4-dimethylphenyl), —NHCO(2,5-dimethylphenyl), —NHCO(2-methylthiophen-5-yl), —CH₂CONH(CH₂)₂(4-methoxyphenyl), —CH₂CONHCH₂(4-chlorophenyl), —CH₂CON(CH₃)₂, —CH₂CO(morpholin-4-yl, —CH₂CON(CH₃)(phenyl), —CH₂CONH(CH₂)₂phenyl, or —CH₂CONH(4-chlorophenyl). Most preferably, R⁵ is —NHCOR⁶, —CONHR⁶, or —NHSO₂R⁶ where R⁶ is alkyl, hydroxyalkyl, optionally substituted phenyl, optionally substituted heteroaryl, optionally substituted heterocycloalkyl, optionally substituted phenylalkyl, optionally substituted phenoxyalkyl, or optionally substituted heteroaralkyl. Most preferably, R⁵ is —NHSO₂phenyl, —NHSO₂(4-methoxyphenyl), —NHSO₂(4-chlorophenyl), —NHCO(phenyl), —NHCO(2,4-dichlorophenyl), —NHCO(4-methylphenyl), —NHCO(3,4-methlenedioxyphenyl), —NHCO(4-methoxyphenyl), —NHCO(4-chlorophenyl), —NHCO(4-methoxy-2-methylphenyl), —NHCO(2,4-dimethylphenyl), —NHCO(3,4-dichlorophenyl), —NHCO(3,4-dimethoxyphenyl), —NHCO(4-ethoxyphenyl), —NHCO(4-fluorophenyl), —NHCO(2,4-difluorophenyl), —NHCO(4-dimethylaminophenyl), or —NHSO₂(benzyl).
(ii) Within this preferred group (A), another even more preferred group of compounds is that wherein Ar¹ is phenylene, Ar² is phenyl; R⁴ is alkyl, halo, alkoxy, —NHCOR⁶ (where R⁶ is optionally substituted phenyl) or —CONR⁶R¹⁰ (where R⁶ is hydrogen, alkyl, optionally substituted phenyl, optionally substituted phenylalkyl or hydroxyalkyl and R¹⁰ is hydrogen, alkyl, alkoxy, carboxyalkyl, hydroxyalkyl, aminocarbonylalkyl, or aminoalkyl), and is at the 3-position of the phenyl ring; and R⁵ is alkyl, halo, alkoxy, carboxy, —CONR⁶R¹⁰ (where R⁶ is hydrogen, alkyl, optionally substituted phenyl, optionally substituted phenylalkyl or hydroxyalkyl and R¹⁰ is hydrogen, alkyl, alkoxy, carboxyalkyl, hydroxyalkyl, aminocarbonylalkyl, or aminoalkyl), —COR⁶ (where R⁶ is optionally substituted heterocycloalkyl), or —NHSO₂R⁶ (where R⁶ is optionally substituted phenyl) and is at the 5-position of the phenyl(Ar²) ring provided that at least one of R⁴ and R⁵ is —NHCOR⁶, —CONR⁶R¹⁰, —CONR⁶R¹⁰, —COR⁶ or —NHSO₂R⁶. Preferably the Ar² ring is 5-(benzylaminocarbonyl)-3-(phenylcarbonylamino)phenyl, 5-(carboxy)-3-(phenylcarbonylamino)phenyl, 5-(morpholin-4-ylcarbonyl)-3-(phenylcarbonylamino)phenyl, 5-(N,N-dimethylaminocarbonyl)-3-(4-methylphenylcarbonylamino)phenyl, 5-(morpholin-4ylcarbonyl)-3-(4-methylphenylcarbonylamino)phenyl, 5-(phenylaminocarbonyl)-3-(4-methyl-phenylcarbonylamino)phenyl, 5-(N,N-dimethylaminocarbonyl)-3-(4-methoxyphenylcarbonyl-amino)phenyl, 5-(morpholin-4-ylcarbonyl)-3-(4-methoxyphenyl-carbonylamino)phenyl, 5-(phenylaminocarbonyl)-3-(4-methoxyphenylcarbonylamino)phenyl, 5-(N,N-dimethyl-aminocarbonyl)-3-(3,4-dimethoxyphenylcarbonylamino)phenyl, 5-(N,N-dimethyl-aminocarbonyl)-3-(phenylcarbonylamino)phenyl, 5-(morpholin-4-ylcarbonyl)-3-(3,4-dimethoxyphenylcarbonylamino)phenyl, 5-(phenylaminocarbonyl)-3-(3,4-dimethoxyphenylcarbonylamino)phenyl, 5-(piperidin-1-ylcarbonyl)-3-(4-methylphenyl-carbonylamino)phenyl, 5-(piperazin-1-ylcarbonyl)-3-(4-methylphenylcarbonylamino)phenyl, 5-(N-methylaminocarbonyl)-3-(4-methylphenylcarbonylamino)phenyl, 5-(N-carboxymethyl-N-methylaminocarbonyl)-3-(4-methylphenylcarbonylamino)phenyl, 5-(N-aminocarbonyl-methylaminocarbonyl)-3-(4-methylphenylcarbonylamino)-phenyl, 5-[N-(2-N-methylamino-ethyl)-N-methylaminocarbonyl]-3-(4-methylphenylcarbonylamino)]-phenyl, 5-(N-carboxy-methylaminocarbonyl)-3-(4-methylphenylcarbonylamino)-phenyl, 5-(N,N-dimethyl-aminocarbonyl)-3-(3,4-methylenephenylcarbonylamino)-phenyl, 5-(morpholin-4-yl-carbonyl)-3-(3,4-methylenephenylcarbonylamino)-phenyl, 5-(phenylaminocarbonyl)-3-(3,4-methylene-phenylcarbonylamino)-phenyl, 5-(N,N-dimethylaminocarbonyl)-3-(2,4-dichloro-phenylcarbonylamino)-phenyl, 5-(phenylaminocarbonyl)-3-(2,4-dichlorophenyl-carbonylamino)-phenyl, 5-(N,N-dimethylaminocarbonyl)-3-(4-chlorophenylcarbonylamino)-phenyl, 5-(morpholin-4-ylcarbonyl)-3-(4-chlorophenylcarbonylamino)-phenyl, 5-(N-ethyl-N-methyl-aminocarbonyl)-3-(4-methylphenylcarbonylamino)-phenyl, 5-(4-methylpiperazin-1-ylcarbonyl)-3-(4-methylphenylcarbonylamino)-phenyl, 5-(3-(RS)-aminocarbonylpiperidin-1-ylcarbonyl)-3-(4-methylphenylcarbonylamino)-phenyl, 5-(N,N-diethylaminocarbonyl)-3-(4-methylphenyl-carbonylamino)-phenyl, 5-(2-N-methylaminoethylaminocarbonyl)-3-(4-methylphenyl-carbonylamino)-phenyl, 5-(4-hydroxypiperidin-1-ylcarbonyl)-3-(4-methylphenyl-carbonylamino)-phenyl, 5-(pyrrolidin-1-ylcarbonyl)-3-(4-methylphenyl-carbonylamino)-phenyl, 5-(3-(RS)-hydroxymethylpiperidin-1-ylcarbonyl)-3-(4-methyl-phenylcarbonylamino)-phenyl, 5-(2-(RS)-hydroxymethylpiperidin-1-ylcarbonyl)-3-(4-methylphenylcarbonylamino)-phenyl, 5-(N-2-hydroxyethyl-N-methylaminocarbonyl)-3-(4-methylphenylcarbonylamino)-phenyl, 5-(N-4-chlorobenzyllaminocarbonyl)-3-(4-methyl-phenylcarbonylamino)-phenyl, 5-(N-3-methylbutylaminocarbonyl)-3-(4-methylphenyl-carbonylamino)-phenyl, 5-(N-2-phenylethylaminocarbonyl)-3-(4-methylphenylcarbonyl-amino)phenyl, 5-[N-2-(4-methoxyphenyl)ethylaminocarbonyl)]-3-(4-methylphenylcarbonyl-amino)-phenyl, 5-(N-methyl-N-methoxyaminocarbonyl)-3-(4-methylphenylcarbonyl-amino)phenyl, 5-(N-methyl-N-phenylaminocarbonyl)-3-(4-methylphenylcarbonylamino)-phenyl, 5-(N-benzylaminocarbonyl)-3-(4-methylphenylcarbonylamino)-phenyl, 5-(N-2-hydroxyethylaminocarbonyl)-3-(4-methylphenylcarbonylamino)-phenyl, 5-[N,N-bis(2-hydroxyethyl)aminocarbonyl]-3-(4-methylphenylcarbonylamino)-phenyl, 5-(4-(RS)-hydroxymethylpiperidin-1-ylcarbonyl)-3-(4-methylphenylcarbonylamino)-phenyl, 5-(3-(RS)-methoxycarbonylpiperidin-1-ylcarbonyl)-3-(4-methylphenylcarbonylamino)-phenyl, 5-(4-(RS)-aminocarbonylpiperidin-1-ylcarbonyl)-3-(4-methylphenyl-carbonylamino)-phenyl, 5-(2-(S)-aminocarbonylpyrrolidin-1-ylcarbonyl)-3-(4-methylphenyl-carbonylamino)-phenyl, 5-(4-(RS)-methoxycarbonylpiperidin-1-ylcarbonyl)-3-(4-methyl-phenylcarbonylamino)-phenyl, 5-[N-(dimethylaminocarbonylmethyl)-N-(methyl)amino-carbonyl]-3-(4-methyl-phenylcarbonylamino)-phenyl, 5-[N-(aminocarbonylmethyl)-N-(methyl)aminocarbonyl]-3-(4-methylphenylcarbonylamino)-phenyl, 5-[N-(methylamino-carbonylmethyl)amino-carbonyl]-3-(4-methylphenylcarbonylamino)-phenyl, 5-[N-[(2-hydroxy)-1-hydroxymethyl)ethyl]-aminocarbonyl-3-(4-methylphenylcarbonylamino)-phenyl, 3-(2,4-dichlorophenylcarbonyl-amino)-5-(piperidin-1-ylcarbonyl)-phenyl, 3-(2,4-dichloro-phenylcarbonyl-amino)-5-(pyrrolidin-1-ylcarbonyl)-phenyl, 3-(2,4-dichlorophenylcarbonyl-amino)-5-(2S-hydroxymethylpyrrolidin-1-ylcarbonyl)-phenyl, or 3-(2,4-dichlorophenyl-carbonyl-amino)-5-(2R-hydroxymethylpyrrolidin-1-ylcarbonyl)-phenyl. Most preferably Ar² is 5-(N,N-dimethylaminocarbonyl)-3-(4-methoxyphenylcarbonylamino)phenyl, 5-(phenylaminocarbonyl)-3-(4-methoxyphenylcarbonyl-amino)phenyl, 5-(piperidin-1-ylcarbonyl)-3-(4-methylphenylcarbonylamino)phenyl, 5-(morpholin-4-ylcarbonyl)-3-(2,4-dichlorophenylcarbonylamino)phenyl, 5-(2-hydroxymethylpiperidin-1-ylcarbonyl)-3-(4-methylphenylcarbonylamino)phenyl, 5-(N,N-dimethylaminocarbonyl)-3-(4-methylphenyl-carbonylamino)phenyl, 5-(N-methoxy-N-methylaminocarbonyl)-3-(4-methylphenyl-carbonylamino)phenyl, 5-(pyrrolidin-1-ylcarbonyl)-3-(2,4-dichlorophenylcarbonylamino)-phenyl, 5-(piperidin-1-ylcarbonyl)-3-(2,4-dichlorophenylcarbonylamino)phenyl, 5-(N,N-dimethylaminocarbonyl)-3-(4-methylphenyl-carbonylamino)phenyl, 5-(N,N-dimethyl-aminocarbonyl)-3-(3,4-dimethoxyphenylcarbonyl-amino)phenyl, 5-(N,N-dimethyl-aminocarbonyl)-3-(2,4-dichlorophenylcarbonyl amino)phenyl, 5-(NN-dimethylaminocarbonyl)-3-(4-chlorophenylcarbonylamino)phenyl, 5-(N-ethyl-N-methylaminocarbonyl)-3-(4-methylphenylcarbonylamino)phenyl, 5-(N,N-diethyl-aminocarbonyl)-3-(4-methylphenylcarbonylamino)phenyl, or 5-(piperidin-1-ylcarbonyl)-3-(4-methylphenylcarbonylamino)phenyl.
   (B). Another preferred group of compounds of Formula I is those wherein:

Ar¹ is phenylene and Ar² is benzimidazol-2-yl or heterocycloalkyl and R¹ is hydrogen.

(C). Yet another preferred group of compounds of Formula I is that wherein:
R¹ and R² are hydrogen, Ar² is located at the 4-position of the Ar¹ ring; R³ and R⁴ are hydrogen; and R⁵ is located at the 3-position of the Ar² ring, the ring atom attaching the Ar² ring to the Ar¹ ring being the 1-position and is cyano, optionally substituted phenyl, optionally substituted heteroaryl, —X—R⁶ [where X is —O—, —NH—, —SO₂—, —CO—, —NR⁹CO—, —CONR¹⁰—, —NR¹¹SO₂— or —NR¹³CONR¹⁴— where R⁹, R¹¹, R¹³ and R¹⁴ are hydrogen and R¹⁰ is hydrogen, alkyl or haloalkyl; and R⁶ is hydrogen (when X is not —O— or —NH—), alkyl (when X is not —O— or —NH—), hydroxyalkyl, optionally substituted phenyl, optionally substituted heteroaryl, optionally substituted heterocycloalkyl, optionally substituted phenylalkyl, optionally substituted phenoxyalkyl, or optionally substituted heteroaralkyl), or —(C₁₋₆alkylene)-Y—R⁷ [where Y is —O—, —CO— or —CONR¹⁰ and R⁷ is hydrogen, alkyl, hydroxyalkyl, optionally substituted phenyl, optionally substituted heteroaryl, optionally substituted heterocycloalkyl, optionally substituted phenylalkyl, optionally substituted phenoxyalkyl, or optionally substituted heteroaralkyl and R¹⁰ is hydrogen or alkyl]. More preferably R⁵ is —X—R⁶ (where X is —NHCO—, —CONR¹⁰—, or —NHSO₂— where R¹⁰ is hydrogen, alkyl or haloalkyl; and R⁶ is hydrogen, alkyl, hydroxyalkyl, optionally substituted phenyl, optionally substituted heteroaryl, optionally substituted heterocycloalkyl, optionally substituted phenylalkyl, optionally substituted phenoxyalkyl, or optionally substituted heteroaralkyl).

Preferably Ar¹ is phenylene and Ar² is phenyl and R⁵ is —NHCOR⁶, —CONHR⁶, or —NHSO₂R⁶ where R⁶ is alkyl, hydroxyalkyl, optionally substituted phenyl, optionally substituted heteroaryl, optionally substituted heterocycloalkyl, optionally substituted phenylalkyl, optionally substituted phenoxyalkyl, or optionally substituted heteroaralkyl.

Most preferably R⁵ is —NHSO₂CH₃, —SO₂CH₃, thiophen-3-yl, cyano, —NHSO₂phenyl, hydroxymethyl, —NHSO₂(3-chlorophenyl), —NHSO₂(4-fluorophenyl), —NHSO₂(3,4-dichlorophenyl), —NHCOphenyl, —NHSO₂(benzyl), —NHSO₂(4chlorophenyl), —NHSO₂(3-trifluoromethylphenyl), —NHSO₂(4-methoxyphenyl), —NHCO(3,4dichlorophenyl), —NHCONH(3-methoxyphenyl), —NHCO(3,4-dimethoxyphenyl), —NHSO₂(2,5-dimethoxyphenyl), —NHSO₂(4trifluoromethoxyphenyl), —NHCO(3-fluorophenyl), —NHCO(2,4 dichlorophenyl), —CONHphenyl, —NHCO(4-methylphenyl), —NHCO(3-trifluoromethylphenyl), -Obenzyl, —O-(3-methoxybenzyl), —NHCO(3,4-methylenedioxyphenyl), —NHCO(4-methoxyphenyl), —CONH(benzyl), —CONH(3,3,3-trifluoroethyl), —NHCO(3-methylbutyl), —CONH(CH₃), —NHCOCH(CH₃)₂, —NHCO(benzyl), —NHCO(CH₂)₂phenyl, —NHCO(CH₂)₂(4-trifluorophenyl), —NHCO(CH₂)₂(4-methoxyphenyl), —NHCO(3,5-dichlorophenyl), —NHCO(CH₂O)phenyl, —NHCO(3-methylbutyl), —NHCOCH2thiophen-2-yl, —NHCO(CH₂)₂(2,4-dichlorophenyl), —NHCO(CH₂)₂(3,4-methylenedioxyphenyl), —NHCO(4-trifluoromethyl-phenyl), —NHCO(4-ethoxyphenyl), —NHCO(4-dimethylaminophenyl), —NHCO(4-fluoro-phenyl), —NHCO(2,4-difluorophenyl), —NHCO(4-chlorophenyl), —CON(CH₃)₂, —NHCO(4-isopropylphenyl), —NHCO(4-trifluoromethoxyphenyl), —NHCO(3-fluoro-4-methoxyphenyl), —NHCO(4-methoxy-2-methylphenyl), —NHCO(2,4-dimethoxyphenyl), —NHCO(4-chloro-2-methoxyphenyl), —NHCO(pyridin-4-yl), —NHCO(pyridin-3-yl), —COmorpholin-4-yl, —CON(CH₃)(phenyl), —CONH(4-chlorophenyl), —CONH(CH₂)₂(4-methoxyphenyl), —CONH(4-chlorobenzyl), —CONH(CH₂)₂phenyl, —CONH(CH₂)₂OH, —COpiperidin-1-yl, —NHCO(2-methyl-phenyl), —NHCO(2,4-dimethylphenyl), —NHCO(2,5-dimethylphenyl), —NHCO(2-methyl-thiophen-5-yl), —CH₂CONH(CH₂)₂(4-methoxyphenyl), —CH₂CONHCH₂(4-chlorophenyl), —CH₂CON(CH₃)₂, —CH₂CO(morpholin-4-yl, —CH₂CON(CH₃)(phenyl), —CH₂CONH(CH₂)₂ phenyl, or —CH₂CONH(4-chlorophenyl). Particularly preferably R⁵ is —NHSO₂phenyl, —NHSO₂(4-methoxyphenyl), —NHSO₂(4-chlorophenyl), —NHCO(phenyl), —NHCO(2,4-dichlorophenyl), —NHCO(4-methylphenyl), —NHCO(3,4-methlenedioxyphenyl), —NHCO(4-methoxyphenyl), —NHCO(4-chlorophenyl), —NHCO(4-methoxy-2-methylphenyl), —NHCO(2,4-dimethylphenyl), —NHCO(3,4-dichlorophenyl), —NHCO(3,4-dimethoxyphenyl), —NHCO(4-ethoxyphenyl), —NHCO(4-fluorophenyl), —NHCO(2,4-difluorophenyl), —NHCO(4-dimethyl-aminophenyl), or —NHSO₂(benzyl).

(D). Yet another preferred group of compounds of Formula I is that wherein:

R¹ and R² are hydrogen, Ar² is located at the 4-position of the Ar¹ ring; R³ is hydrogen, R⁴ is located at the 3-position and R⁵ at the 5-position of the Ar² ring, the ring atom attaching the Ar² ring to the Ar¹ ring being the 1-position. Preferably, R⁴ is alkyl, halo, alkoxy, —NHCOR⁶ (where R⁶ is optionally substituted phenyl) or —CONR⁶R¹⁰ (where R⁶ is hydrogen, alkyl, optionally substituted phenyl, optionally substituted phenylalkyl or hydroxyalkyl and R¹⁰ is hydrogen, alkyl, alkoxy, carboxyalkyl, hydroxyalkyl, aminocarbonylalkyl, or aminoalkyl); and R⁵ is alkyl, halo, alkoxy, carboxy, —CONR⁶R¹⁰ (where R⁶ is hydrogen, alkyl, optionally substituted phenyl, optionally substituted phenylalkyl or hydroxyalkyl and R¹⁰ is hydrogen, alkyl, alkoxy, carboxyalkyl, hydroxyalkyl, aminocarbonylalkyl, or aminoalkyl), —COR⁶ (where R⁶ is optionally substituted heterocycloalkyl), or —NHSO₂R⁶ (where R⁶ is optionally substituted phenyl) provided that at least one of R⁴ and R⁵ is other than alkyl, halo, alkoxy, or carboxy when Ar¹ is phenylene and Ar² is phenyl. Preferably, Ar¹ is phenylene and Ar² is phenyl.

Most preferably the Ar² ring is 5-(benzylaminocarbonyl)-3-(phenylcarbonylamino)-phenyl, 5-(carboxy)-3-(phenylcarbonylamino)phenyl, 5-(morpholin-4-ylcarbonyl)-3-(phenylcarbonylamino)phenyl, 5-(N,N-dimethylaminocarbonyl)-3-(4-methylphenylcarbonyl-amino)phenyl, 5-(morpholin-4-ylcarbonyl)-3-(4-methylphenylcarbonylamino)phenyl, 5-(phenylaminocarbonyl)-3-(4-methyl-phenylcarbonylamino)phenyl, 5-(N,N-dimethylamino-carbonyl)-3-(4-methoxyphenylcarbonyl-amino)phenyl, 5-(morpholin-4-ylcarbonyl)-3-(4-methoxyphenyl-carbonylamino)phenyl, 5-(phenylaminocarbonyl)-3-(4-methoxyphenyl-carbonylamino)phenyl, 5-(N,N-dimethyl-aminocarbonyl)-3-(3,4-dimethoxyphenyl-carbonylamino)phenyl, 5-(N,N-dimethyl-aminocarbonyl)-3-(phenylcarbonylamino)phenyl, 5-(morpholin-4-ylcarbonyl)-3-(3,4-dimethoxyphenylcarbonylamino)phenyl, 5-(phenylaminocarbonyl)-3-(3,4-dimethoxyphenylcarbonylamino)phenyl, 5-(piperidin-1-yl-carbonyl)-3-(4-methylphenyl-carbonylamino)phenyl, 5-(piperazin-1-ylcarbonyl)-3-(4-methylphenylcarbonylamino)phenyl, 5-(N-methylaminocarbonyl)-3-(4-methylphenyl-carbonylamino)phenyl, 5-(N-carboxymethyl-N-methylaminocarbonyl)-3-(4-methylphenyl-carbonylamino)phenyl, 5-(N-aminocarbonyl-methylaminocarbonyl)-3-(4-methylphenyl-carbonylamino)-phenyl, 5-[N-(2-N-methylamino-ethyl)-N-methylaminocarbonyl]-3-(4-methylphenylcarbonylamino)]-phenyl, 5-(N-carboxy-methylaminocarbonyl)-3-(4-methyl-phenylcarbonylamino)-phenyl, 5-(N,N-dimethyl-aminocarbonyl)-3-(3,4-methylene-phenylcarbonylamino)-phenyl, 5-(morpholin-4-yl-carbonyl)-3-(3,4-methylene-phenylcarbonylamino)-phenyl, 5-(phenylaminocarbonyl)-3-(3,4-methylene-phenylcarbonylamino)-phenyl, 5-(N,N-dimethylaminocarbonyl)-3-(2,4-dichloro-phenylcarbonylamino)-phenyl, 5-(phenylaminocarbonyl)-3-(2,4-dichlorophenyl-carbonylamino)-phenyl, 5-(N,N-dimethylaminocarbonyl)-3-(4-chlorophenylcarbonylamino)-phenyl, 5-(morpholin-4-ylcarbonyl)-3-(4-chlorophenylcarbonylamino)-phenyl, 5-(N-ethyl-N-methyl-aminocarbonyl)-3-(4-methylphenylcarbonylamino)-phenyl, 5-(4-methylpiperazin-1-ylcarbonyl)-3-(4-methylphenylcarbonylamino)-phenyl, 5-(3-(RS)-aminocarbonylpiperidin-1-ylcarbonyl)-3-(4-methylphenylcarbonylamino)-phenyl, 5-(N,N-diethylaminocarbonyl)-3-(4-methylphenyl-carbonylamino)-phenyl, 5-(2-N-methylaminoethylaminocarbonyl)-3-(4-methylphenyl-carbonylamino)-phenyl, 5-(4-hydroxypiperidin-1-ylcarbonyl)-3-(4-methyl-phenyl-carbonylamino)-phenyl, 5-(pyrrolidin-1-ylcarbonyl)-3-(4-methylphenylcarbonyl-amino)-phenyl, 5-(3-(RS)-hydroxymethylpiperidin-1-ylcarbonyl)-3-(4-methylphenyl-carbonylamino)-phenyl, 5-(2-(RS)-hydroxymethylpiperidin-1-ylcarbonyl)-3-(4-methylphenylcarbonylamino)-phenyl, 5-(N-2-hydroxyethyl-N-methylaminocarbonyl)-3-(4methylphenylcarbonylamino)-phenyl, 5-(N-4-chlorobenzyllaminocarbonyl)-3-(4-methylphenylcarbonylamino)-phenyl, 5-(N-3-methylbutylaminocarbonyl)-3-(4-methylphenylcarbonylamino)-phenyl, 5-(N-2-phenylethylaminocarbonyl)-3-(4-methylphenylcarbonylamino)-phenyl, 5-[N-2-(4-methoxyphenyl)ethylaminocarbonyl)]-3-(4-methylphenylcarbonylamino)-phenyl, 5-(N-methyl-N-methoxyaminocarbonyl)-3-(4-methylphenylcarbonyl-amino)phenyl, 5-(N-methyl-N-phenylaminocarbonyl)-3-(4-methylphenylcarbonylamino)-phenyl, 5-(N-benzylaminocarbonyl)-3-(4-methylphenylcarbonylamino)-phenyl, 5-(N-2-hydroxyethylaminocarbonyl)-3-(4-methylphenylcarbonylamino)-phenyl, 5-[N,N-bis(2-hydroxyethyl)aminocarbonyl]-3-(4-methylphenylcarbonylamino)-phenyl, 5-(4-(RS)-hydroxymethylpiperidin-1-ylcarbonyl)-3-(4-methylphenylcarbonylamino)-phenyl, 5-(3-(RS)-methoxycarbonylpiperidin-1-yl-carbonyl)-3-(4-methylphenylcarbonylamino)-phenyl, 5-(4-(RS)-aminocarbonylpiperidin-1-ylcarbonyl)-3-(4-methylphenyl-carbonylamino)-phenyl, 5-(2-(S)-aminocarbonylpyrrolidin-1-ylcarbonyl)-3-(4-methylphenylcarbonylamino)-phenyl, 5-(4-(RS)-methoxycarbonylpiperidin-1-ylcarbonyl)-3-(4-methylphenylcarbonylamino)-phenyl, 5-[N-(dimethylamino-carbonylmethyl)-N-(methyl)aminocarbonyl]-3-(4-methyl-phenylcarbonylamino)-phenyl, 5-[N-(aminocarbonylmethyl)-N-(methyl)aminocarbonyl]-3-(4-methylphenylcarbonylamino)-phenyl, 5-[N-(methylaminocarbonylmethyl)amino-carbonyl]-3-(4-methylphenylcarbonyl-amino)-phenyl, 5-[N-[(2-hydroxy)-1-hydroxymethyl)ethyl]-aminocarbonyl-3-(4-methyl-phenylcarbonylamino)-phenyl, 3-(2,4-dichlorophenylcarbonyl-amino)-5-(piperidin-1-yl-carbonyl)-phenyl, 3-(2,4-dichlorophenylcarbonyl-amino)-5-(pyrrolidin-1-ylcarbonyl)-phenyl, 3-(2,4-dichlorophenylcarbonyl-amino)-5-(2S-hydroxymethylpyrrolidin-1-ylcarbonyl)-phenyl, or 3-(2,4-dichlorophenylcarbonyl-amino)-5-(2R-hydroxymethylpyrrolidin-1-yl-carbonyl)-phenyl. Particularly preferably Ar² is 5-(N,N-dimethylaminocarbonyl)-3-(4-methoxyphenylcarbonylamino)phenyl, 5-(phenylaminocarbonyl)-3-(4-methoxyphenyl-carbonyl-amino)phenyl, 5-(piperidin-1-ylcarbonyl)-3-(4-methylphenylcarbonyl-amino)phenyl, 5-(morpholin-4-ylcarbonyl)-3-(2,4-dichlorophenylcarbonylamino)phenyl, 5-(2-hydroxymethylpiperidin-1-ylcarbonyl)-3-(4-methylphenylcarbonylamino)phenyl, 5-(N,N-dimethylaminocarbonyl)-3-(4-methylphenylcarbonylamino)phenyl, 5-(N-methoxy-N-methylaminocarbonyl)-3-(4-methylphenylcarbonylamino)phenyl, 5-(pyrrolidin-1-yl-carbonyl)-3-(2,4-dichlorophenylcarbonylamino)phenyl, 5-(piperidin-1-ylcarbonyl)-3-(2,4-dichlorophenylcarbonylamino)phenyl, 5-(N,N-dimethylaminocarbonyl)-3-(4-methylphenyl-carbonylamino)phenyl, 5-(N,N-dimethylaminocarbonyl)-3-(3,4-dimethoxyphenylcarbonyl-amino)phenyl, 5-(N,N-dimethylaminocarbonyl)-3-(2,4-dichlorophenylcarbonylamino)phenyl, 5-(N,N-dimethylaminocarbonyl)-3-(4-chlorophenylcarbonylamino)phenyl, 5-(N-ethyl-N-methylaminocarbonyl)-3-(4-methylphenylcarbonylamino)phenyl, 5-(N,N-diethylamino-carbonyl)-3-(4methylphenylcarbonylamino)phenyl, or 5-(piperidin-1-ylcarbonyl)-3-(4-methylphenylcarbonylamino)phenyl.
(E) Yet another preferred group of compounds of Formula I is represented by Formula Ib: 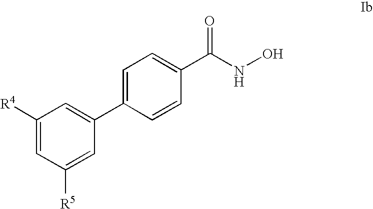
wherein:
R⁴ is hydrogen, alkyl, halo, haloalkyl, cyano, carboxy, carboxyalkyl, alkoxycarbonyl, heterocycloaminoalkyl, —X—R⁶, or —(C₁₋₆alkylene)-Y—R⁷ where X and Y are independently —O—, —S—, —SO—, —SO₂—, —NR⁸—, —CO—, —NR⁹CO—, —CONR¹⁰—, —NR¹¹SO₂—, —SO₂NR¹²—, —NHC(O)O—, —OC(O)NH—, —NR¹³CONR¹⁴—, or —NR¹⁵SO₂NR¹⁶—;
R⁵ is —X—R⁶ or —(C₁₋₆alkylene)-Y—R⁷ where X and Y are independently —O—, —S—, —SO—, —SO₂—, —NR⁸—, —CO—, —NR⁹CO—, —CONR¹⁰—, —NR¹¹SO₂—, —SO₂NR¹²—, —NHC(O)O—, —OC(O)NH—, —NR¹³CONR¹⁴—, or —NR¹⁵SO₂NR¹⁶— where:
each R⁶ and R⁷ is independently hydrogen, alkyl, hydroxyalkyl, optionally substituted phenyl, optionally substituted heteroaryl, optionally substituted heterocycloalkyl, cycloalkyl, optionally substituted phenylalkyl, optionally substituted phenyalkenyl, optionally substituted phenoxyalkyl, optionally substituted phenylaminoalkyl, optionally substituted heteroaralkyl, optionally substituted heteroaryloxyalkyl, optionally substituted heterocycloalkylalkyl, or cycloalkylalkyl,
each R⁸, R⁹, R¹¹, R¹³, and R¹⁵ is independently hydrogen, alkyl, hydroxyalkyl, alkoxyalkyl, or optionally substituted phenylalkyl; and
each R¹⁰, R¹², R¹⁴, and R¹⁶ is independently hydrogen, alkyl, optionally substituted phenylalkyl, alkoxy, hydroxyalkyl, haloalkyl, alkoxyalkyl, carboxyalkyl, cyanoalkyl, aminoalkyl, aminocarbonylalkyl, alkylaminocarbonylalkyl, dialkylaminocarbonylalkyl, or acyl;
and individual stereoisomers, mixtures of stereoisomers; or a pharmaceutically acceptable salt thereof provided that (i) when one of R⁴ and R⁵ is methoxy, then the other of R⁴ and R⁵ is not —OR⁶ where R⁶ is cyclopentyl or phenylpentyl.

Preferably, (i) when R⁴ is hydrogen, alkyl, halo, haloalkyl, cyano, carboxy, alkoxycarbonyl, or —X—R⁶ [where X is —O—, —S—, —SO—, —NR⁸—, or —CO— where R⁶ and R⁸ are independently hydrogen or alkyl], then R⁵ is not —X—R⁶ [where X is —O—, —S—, —SO—, or —NR⁸— where R⁶ and R⁸ are independently hydrogen or alkyl]; (ii) when R⁴ and/or R⁵ are —X—R⁶ [where X is —CONR¹⁰—, —SO₂NR¹²—, —NR¹³CONR¹⁴—, or —NR¹⁵SO₂NR¹⁶—, then both R⁶ and R¹⁰, R¹², R¹⁴, and R¹⁶ are not simultaneously hydrogen; and (iii) when R⁴ and/or R⁵ are —X—R⁶ where X is —NR⁹CO—, —NR¹¹SO₂—, —NHC(O)O—, or —OC(O)NH—, then R⁶ is not hydrogen.
(F). Yet another preferred group of compounds of Formula I is represented by Formula Ic: 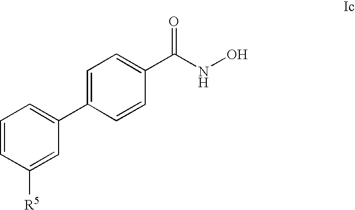
wherein:
R⁵ is —X—R⁶ or —(C₁₋₆alkylene)-Y—R⁷ where:
   X is —NR⁸—, —NR⁹CO—, —NR¹¹SO₂—, —NR¹³CONR¹⁴—, or —NR¹⁵SO₂NR¹⁶—;
   Y is —O—, —S—, —SO—, —SO₂—, —NR⁸—, —CO—, —NR⁹CO—, or —CONR¹⁰—;
   R⁶ is alkyl, optionally substituted phenyl, optionally substituted heteroaryl, optionally substituted phenylalkyl, optionally substituted phenoxyalkyl, optionally substituted heteroaralkyl, or optionally substituted phenylalkenyl;
   R⁷ is hydrogen, alkyl, hydroxyalkyl, optionally substituted phenyl, optionally substituted heteroaryl, optionally substituted heterocycloalkyl, cycloalkyl, optionally substituted phenylalkyl, optionally substituted phenoxyalkyl, optionally substituted heteroaralkyl, optionally substituted heteroaryloxyalkyl, optionally substituted heterocycloalkylalkyl, or cycloalkylalkyl;
   R⁸, R⁹, R¹¹, R¹³, and R¹⁵ are independently hydrogen, alkyl, hydroxyalkyl, alkoxyalkyl, or optionally substituted phenylalkyl; and
   R¹⁰, R¹⁴, and R¹⁶ are independently hydrogen, alkyl, optionally substituted phenylalkyl, alkoxy, hydroxyalkyl, or alkoxyalkyl; or
      a pharmaceutically acceptable salt thereof provided that when R⁵ is —NR⁶R⁸ then R⁶ is not alkyl.

Preferably, in compounds of Formula Ic, X is —NR⁸—, —NR⁹CO—, —NR¹¹SO₂—, or —NR¹³CONR¹⁴—; Y is —O—, —NR⁸—, —CO—, —NR⁹CO—, or —CONR¹⁰—; R⁶ is alkyl, optionally substituted phenyl, optionally substituted heteroaryl, optionally substituted phenylalkyl, optionally substituted phenoxyalkyl, or optionally substituted heteroaralkyl; R⁷ is alkyl, optionally substituted phenyl, optionally substituted heterocycloalkyl, optionally substituted phenylalkyl, optionally substituted heteroaryl, or optionally substituted heteroaralkyl; R⁸, R⁹, R¹¹, and R¹³ are independently hydrogen, alkyl, hydroxyalkyl, or alkoxyalkyl; and R¹⁰ and R¹⁴ are independently hydrogen or alkyl. More preferably, R⁵ is methylsulfonylamino, phenylsulfonylamino, phenylureido, 3-chlorophenylsulfonylamino, 4-fluorophenylsulfonylamino, 3,4-dichlorophenylsulfonylamino, phenylcarbonylamino, benzylsulfonylamino, 4-chlorophenylsulfonylamino, 3-trifluoromethylphenylsulfonylamino, 4-methoxyphenylsulfonylamino, 3,4-dichlorophenylcarbonylamino, 3-methoxyphenylureido, 3,4-dimethoxyphenylcarbonylamino, 2,5-dimethoxyphenylsulfonylamino, 4-trifluoromethox-yphenylsulfonylamino, 3-fluorophenylcarbonylamino, 2,4-dichlorophenylcarbonylamino, 4-methylphenylcarbonylamino, 3-trifluoromethylphenylcarbonylamino, 3,4-methylenedioxyphenylcarbonylamino, 4-methoxyphenylcarbonylamino, 2-propylcarbonyl-amino, benzylcarbonylamino, 2-phenylethylcarbonylamino, 2-(4-trifluoromethylphenyl)-ethylcarbonylamino, 2-(4-methoxyphenyl)ethylcarbonylamino, 3,5-dichlorophenylcarbonyl-amino, phenoxymethylcarbonylamino, 3-methylbutylcarbonylamino, thiophen-2-ylmethyl-carbonylamino, 2-(2,4-dichlorophenyl)ethylcarbonylamino, 2-(3,4-methylenedioxyphenyl)-ethylcarbonylamino, 4-trifluoromethylphenylcarbonylamino, 4-ethoxyphenylcarbonylamino, 4-dimethylaminophenylcarbonylamino, 4-fluorophenylcarbonylamino, 2,4-difluorophenyl-carbonylamino, 4-chlorophenylcarbonylamino, 4-isopropylphenylcarbonylamino, 4-trifluoromethoxyphenylcarbonylamino, 3-fluoro-4-methoxyphenylcarbonylamino, 4-methoxy-2-methylphenylcarbonylamino, 2,4-dimethoxyphenylcarbonylamino, 4-chloro-2-methoxyphenylcarbonylamino, pyridin-4-ylcarbonylamino, pyridin-3-ylcarbonylamino, 2-methylphenylcarbonylamino, 2,4-dimethylphenylcarbonylamino, 2,5-dimethylphenyl-carbonylamino, 2-methylthiophen-5-ylcarbonylamino, benzylamino, 4-methoxybenzylamino, 4-methylbenzylamino, 2-(4-methoxyphenyl)ethylaminocarbonylmethyl, 4-chlorobenzyl-aminocarbonylmethyl, dimethylaminocarbonylmethyl, morpholin-4-ylcarbonylmethyl, N-benzyl-N-methylaminocarbonylmethyl, 2-(phenyl)ethylaminocarbonylmethyl, 4-chlorophenylaminocarbonylmethyl, phenylcarbonylaminomethyl, pyridin-3-ylmethyl-carbonylaminomethyl, N-benzoyl-N-(2-hydroxyethyl)aminomethyl, N-benzyl-N-(2-hydroxyethyl)aminomethyl, N-benzyl-N-(2-methoxyethyl)aminomethyl, benzylaminomethyl, 2-indol-3-ylethylaminomethyl, 3,4-methylenedioxyphenylmethylaminomethyl, pyridin-4yl-methylaminomethyl, pyridin-3-yloxymethyl, 2-pyridin-3-ylethylaminomethyl, phenoxymethyl, 4-methylphenoxymethyl, 4-chlorophenoxymethyl, 3-phenylpropylaminomethyl, phenylaminomethyl, 4-methylphenylaminomethyl, or 4-chlorophenylaminomethyl. Most preferably, R⁵ is phenylsulfonylamino, 4-methoxyphenylsulfonylamino, 4-methylphenyl-carbonylamino, or 4-methoxy-2-methylphenylcarbonylamino.
(G) Yet another preferred group of compounds of Formula I is represented by Formula Id: 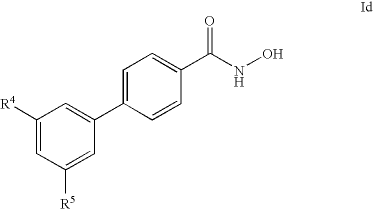
wherein:
R⁴ is carboxy, heterocycloaminoalkyl, —CO-(optionally substituted heterocycloalkyl), —CONR⁶R¹⁰, or —(C₁₋₄alkylene)-Y—R⁷ where Y is —NR⁸— or —O—; R⁶ is hydrogen, alkyl, hydroxyalkyl, optionally substituted phenyl, optionally substituted heteroaryl, optionally substituted phenylalkyl, optionally substituted phenoxyalkyl, optionally substituted heteroaralkyl, or optionally substituted phenylalkenyl; R⁷ is alkyl, optionally substituted phenyl, optionally substituted phenylalkyl, or optionally substituted phenylalkenyl; R⁸ is alkyl, optionally substituted phenylalkyl, hydroxyalkyl, or alkoxyalkyl; and R¹⁰ is hydrogen, alkyl, optionally substituted phenylalkyl, alkoxy, hydroxyalkyl, alkoxyalkyl, carboxyalkyl, cyanoalkyl, aminoalkyl, arminocarbonylalkyl, alkylaminocarbonylalkyl, dialkylaminocarbonylalkyl, or acyl; and
R⁵ is —X—R⁶ or —(C₁₋₆alkylene)-Y—R⁷ where X is —NR⁸—, —NR⁹CO—, —NR¹¹SO₂—, —NR¹³CONR¹⁴—, or —NR¹⁵SO₂NR¹⁶—; Y is —O—, —S—, —SO—, —SO₂—, —NR⁸—, —CO—, —NR⁹CO—, or —CONR¹⁰—; R⁶ is alkyl, optionally substituted phenyl, optionally substituted heteroaryl, optionally substituted phenylalkyl, optionally substituted phenoxyalkyl, optionally substituted phenylaminoalkyl, optionally substituted heteroaralkyl, or optionally substituted phenylalkenyl; R⁷ is alkyl, optionally substituted phenyl, optionally substituted phenylalkyl, optionally substituted phenylalkenyl, or optionally substituted heterocycloalkyl; R⁸, R⁹, R¹¹, R¹³, and R¹⁵ are independently hydrogen, alkyl, or optionally substituted phenylalkyl; and R¹⁰, and R¹⁶ are independently hydrogen, alkyl, optionally substituted phenylalkyl, alkoxy, hydroxyalkyl, or alkoxyalkyl;
   or a pharmaceutically acceptable salt thereof provided both R⁶ and R¹⁰ are not simultaneously hydrogen.

More preferably, a compound of Formula Id wherein:
R⁴ is carboxy, heterocycloaminoalkyl, —CO-(optionally substituted heterocycloamino), —CONR⁶R¹⁰, or —(C₁₋₆alkylene)-Y—R⁷ where Y is —NR⁸— or —O—; R⁶ is hydrogen, alkyl, hydroxyalkyl, optionally substituted phenyl, or optionally substituted phenylalkyl; R⁷ is alkyl, optionally substituted phenyl, optionally substituted phenylalkyl, or optionally substituted phenylalkenyl; R⁸ is hydrogen or alkyl; and R¹⁰ is hydrogen, alkyl, alkoxy, hydroxyalkyl, alkoxyalkyl, carboxyalkyl, aminoalkyl, aminocarbonylalkyl, alkylaminocarbonylalkyl, or dialkylaminocarbonylalkyl; and
R⁵ is —X—R⁶ where X is —NHCO—, —NHSO₂—, or —NHCONH— and R⁶ is alkyl, optionally substituted phenyl, optionally substituted heteroaryl, optionally substituted phenylalkyl, optionally substituted phenylaminoalkyl, optionally substituted heteroaralkyl, or optionally substituted phenylalkenyl.

Even more preferably, a compound of Formula Id wherein R⁴ is —CONR⁶R¹⁰ where R⁶ is hydrogen, alkyl, hydroxyalkyl, optionally substituted phenyl, or optionally substituted phenylalkyl; and R¹⁰ is hydrogen, alkyl, alkoxy, hydroxyalkyl, alkoxyalkyl, carboxyalkyl, aminoalkyl, aminocarbonylalkyl, alkylaminocarbonylalkyl, or dialkylaminocarbonylalkyl provided both R⁶ and R¹⁰ are not hydrogen; and
R⁵ is —NHCOR⁶ where R⁶ is optionally substituted phenyl, optionally substituted heteroaryl, optionally substituted phenylalkyl, optionally substituted phenylaminoalkyl, optionally substituted heteroaralkyl, or optionally substituted phenylalkenyl.

Even more preferably, a compound of Formula Id wherein R⁴ is —CO-(optionally substituted heterocycloamino), preferably pyrrolidin-1-yl, piperidin-1-yl, morpholin-4-yl, or piperazin-1-yl wherein said rings are optionally substituted with methoxycarbonyl, aminocarbonyl hydroxymethyl, ethoxycarbonyl, methyl, or hydroxy; and
R⁵ is —NHCOR⁶ where R⁶ is optionally substituted phenyl, optionally substituted heteroaryl, optionally substituted phenylalkyl, optionally substituted phenylaminoalkyl, optionally substituted heteroaralkyl, or optionally substituted phenylalkenyl.

Even more preferably a compound of Formula Id wherein R⁴ is phenylaminocarbonyl, benzylaminocarbonyl, carboxy, dimethylaminocarbonyl, morpholin-4-ylcarbonyl, piperidin-1-ylcarbonyl, piperazin-1-ylcarbonyl, methylaminocarbonyl, N-carboxymethyl-N-methylaminocarbonyl, aminocarbonylmethylaminocarbonyl, 2-N-methylaminoethyl-2N-methylaminocarbonyl, carboxymethylaminocarbonyl, N-ethyl-N-methylaminocarbonyl, 4-methylpiperazin-1-ylcarbonyl, 3-aminocarbonylpiperidin-1-ylcarbonyl, diethylaminocarbonyl, 2-(methylaminoethyl)aminocarbonyl, 4-hydroxypiperidin-1-ylcarbonyl, pyrrolidin-1-ylcarbonyl, 3-hydroxymethylpiperidin-1-ylcarbonyl, 2-hydroxymethylpiperidin-1-ylcarbonyl, N-(2-hydroxyethyl)-N-methylaminocarbonyl, 4-chlorophenylmethyl-aminocarbonyl, 3-methylbutylaminocarbonyl, 2-phenylethylaminocarbonyl, 2-(4methoxyphenyl)ethylaminocarbonyl, N-methoxy-N-methylaminocarbonyl, N-methyl-N-phenylaminocarbonyl, 2-hydroxyethylaminocarbonyl, bis(2-hydroxyethyl)-aminocarbonyl, 4-hydroxymethylpiperidin-1-ylcarbonyl, 3-ethoxycarbonylpiperidin-1-ylcarbonyl, 4-aminocarbonylpiperidin-1-ylcarbonyl, 2S-aminocarbonylpyrrolidin-1-ylcarbonyl, 4-methoxycarbonylpiperidin-1-ylcarbonyl, methylaminocarbonylmethylaminocarbonyl, 4-chlorophenylaminocarbonyl, N-dimethylaminocarbonylmethyl-N-methylaminocarbonyl, N-aminocarbonylmethyl-N-methylaminocarbonyl, 2-hydroxy-1-hydroxymethylethyl-aminocarbonyl, 2S-hydroxymethylpyrrolidin-1-ylcarbonyl, 2R-hydroxymethylpyrrolidin-1-ylcarbonyl, dimethylaminomethyl, propoxymethyl, piperidin-1-ylmethyl, benzyloxymethyl, phenoxymethyl, or 2-phenylpropen-2yloxymethyl, pyrrolidin-1-ylmethyl. Even more preferably, R⁴ is dimethylaminocarbonyl, piperidin-1-ylcarbonyl, N-ethyl-N-methyl-aminocarbonyl, diethylaminocarbonyl, 4-hydroxypiperidin-1-ylcarbonyl, pyrrolidin-1-ylcarbonyl, 3-hydroxymethylpiperidin-1-ylcarbonyl, 2-hydroxymethylpiperidin-1-ylcarbonyl, N-(2-hydroxyethyl)-N-methylaminocarbonyl, or pyrrolidin-1-ylmethyl.

Even more preferably a compound of Formula Id wherein R⁵ is phenylcarbonylamino, phenylsulfonylamino, 4-methylphenylcarbonylamino, 4-methoxyphenylcarbonylamino, 3,4-dimethoxyphenylcarbonylamino, 3,4-methoxylenedioxyphenylcarbonylamino, 2,4-dichlorophenylcarbonylamino, 4-chlorophenylcarbonylamino, benzylcarbonylamino, 2-phenylethylcarbonylamino, 4-methoxy-2-methylphenylcarbonylamino, 2-phenyl-ethenylcarbonylamino, 2,4-dimethylphenylcarbonylamino, 2-propylcarbonylamino, 3-phenylureido, benzylamino, phenylaminomethylcarbonylamino, indol-3-ylmethyl-carbonylamino, 2,4-dichlorobenzylamino, pyridin-4-ylmethylcarbonylamino, or furan-3-ylcarbonylamino. Particularly preferably, R⁵ is 4-methylphenylcarbonylamino, 4-methoxyphenylcarbonylamino, 3,4-dimethoxyphenylcarbonylamino, 3,4-methylene-dioxyphenylcarbonylamino, 2,4-dichlorophenylcarbonylamino, 4-chlorophenylcarbonylamino, 4-methoxy-2-phenylcarbonyl-amino, or 2,4-dimethylphenylcarbonylamino.

Even more preferably a compound of Formula Id wherein R⁴ is dimethylaminocarbonyl, piperidin-1-ylcarbonyl, N-ethyl-N-methylaminocarbonyl, diethylaminocarbonyl, 4-hydroxypiperidin-1-ylcarbonyl, pyrrolidin-1-ylcarbonyl, 3-hydroxymethylpiperidin-1-ylcarbonyl, 2-hydroxymethylpiperidin-1-ylcarbonyl, N-(2-hydroxyethyl)-N-methylaminocarbonyl, or pyrrolidin-1-ylmethyl and R⁵ is 4-methylphenylcarbonylamino, 4-methoxyphenylcarbonylamino, 3,4-dimethoxyphenylcarbonylamino, 3,4-methylenedioxyphenylcarbonylamino, 2,4-dichlorophenylcarbonylamino, 4-chlorophenylcarbonylamino, 4-methoxy-2-phenylcarbonylamino, or 2,4-dimethylphenylcarbonylamino.

In a second aspect, this invention is directed to a pharmaceutical composition comprising a therapeutically effective amount of a compound of Formula I or pharmaceutically acceptable salts thereof and a pharmaceutically acceptable excipient.

In a third aspect, this invention is directed to a method for treating a disease in an animal mediated by HDAC which method comprises administering to the animal a pharmaceutical composition comprising a therapeutically effective amount of a compound of Formula I. 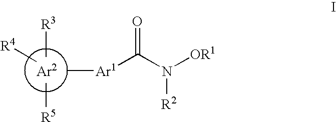
wherein:
R¹ is hydrogen or alkyl;
R² is hydrogen;
Ar¹ is phenylene or a six membered heteroarylene ring containing one or two nitrogen ring atoms, the rest of the ring atoms being carbon; wherein said Ar¹ group is optionally substituted with one or two groups independently selected from alkyl, halo, hydroxy, alkoxy, trifluoromethoxy, or trifluoromethyl;
Ar² is aryl, heteroaryl, cycloalkyl or heterocycloalkyl;
R³ is hydrogen, alkyl, halo, hydroxy, or alkoxy; and
R⁴ and R⁵ are independently selected from the group consisting of hydrogen, alkyl, halo, haloalkyl, nitro, cyano, carboxy, carboxyalkyl, alkoxycarbonyl, optionally substituted phenyl, optionally substituted heteroaryl, optionally substituted heterocycloalkyl, cycloalkyl, heterocycloaminoalkyl, —X—R⁶, or —(C₁₋₆alkylene)-Y—R⁷ where X and Y are independently —O—, —S—, —SO—, —SO₂—, —NR⁸—, —CO—, —NR⁹CO—, —CONR¹⁰—, —NR¹¹SO₂—, —SO₂NR¹²—, —NHC(O)O—, OC(O)NH—, —NR¹³CONR¹⁴—, or —NR¹⁵SO₂NR^{16— where R}⁶ and R⁷ are independently hydrogen, alkyl, hydroxyalkyl, optionally substituted phenyl, optionally substituted heteroaryl, optionally substituted heterocycloalkyl, cycloalkyl, optionally substituted phenylalkyl, optionally substituted phenoxyalkyl, optionally substituted phenylaminoalkyl, optionally substituted heteroaralkyl, optionally substituted heteroaryloxyalkyl, optionally substituted heterocycloalkylalkyl, or cycloalkylalkyl, R⁸, R⁹, R¹¹, R¹³, and R¹⁵ are independently hydrogen, alkyl, hydroxyalkyl, alkoxyalkyl, or optionally substituted phenylalkyl; R¹⁰, R¹², R¹⁴, and R¹⁶ are independently hydrogen, alkyl, optionally substituted phenylalkyl, alkoxy, hydroxyalkyl, haloalkyl, alkoxyalkyl, carboxyalkyl, cyanoalkyl, aminoalkyl, aminocarbonylalkyl, alkylaminocarbonylalkyl, dialkylaminocarbonylalkyl, or acyl or R⁴ and R⁵ together form methylenedioxy; and individual isomers, mixtures of isomers; or a pharmaceutically acceptable salt thereof and a pharmaceutically acceptable excipient. Preferably, a compound of Formula Ia, Ib, Ic, or Id. Preferably, the disease is a proliferative disorder such as cancer and bipolar disorders and the animal is a human. Preferably, the cancer is prostate cancer, breast cancer, lung melanoma, stomach cancer, neuroblastoma, colon cancer, pancreatic cancer, ovarian cancer, and T-cell lymphoma.

In a fourth aspect, this invention is directed to a method for treating cancer in an animal which method comprises administering to the animal a pharmaceutical composition comprising a therapeutically effective amount of a compound of Formula I: 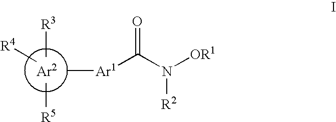
wherein:
R¹ is hydrogen or alkyl;
R² is hydrogen;
Ar¹ is phenylene or a six membered heteroarylene ring containing one or two nitrogen ring atoms, the rest of the ring atoms being carbon; wherein said Ar¹ group is optionally substituted with one or two groups independently selected from alkyl, halo, hydroxy, alkoxy, trifluoromethoxy, or trifluoromethyl;
Ar² is aryl, heteroaryl, cycloalkyl or heterocycloalkyl;
R³ is hydrogen, alkyl, halo, hydroxy, or alkoxy; and
R⁴ and R⁵ are independently selected from the group consisting of hydrogen, alkyl, halo, haloalkyl, nitro, cyano, carboxy, carboxyalkyl, alkoxycarbonyl, optionally substituted phenyl, optionally substituted heteroaryl, optionally substituted heterocycloalkyl, cycloalkyl, heterocycloaminoalkyl, —X—R⁶, or —(C₁₋₆alkylene)-Y—R⁷ where X and Y are independently —O—, —S—, —SO—, —SO₂—, —NR⁸—, —CO—, —NR⁹CO—, —CONR¹⁰—, —NR¹¹SO₂—, —NHC(O)O—, —OC(O)NH—, —NR¹³CONR¹⁴—, or —NR¹⁵SO₂NR¹⁶— where R⁶ and R⁷ are independently hydrogen, alkyl, hydroxyalkyl, optionally substituted phenyl, optionally substituted heteroaryl, optionally substituted heterocycloalkyl, cycloalkyl, optionally substituted phenylalkyl, optionally substituted phenoxyalkyl, optionally substituted phenylaminoalkyl, optionally substituted heteroaralkyl, optionally substituted heteroaryloxyalkyl, optionally substituted heterocycloalkylalkyl, or cycloalkylalkyl, R⁸, R⁹, R¹¹, R¹³, and R¹⁵ are independently hydrogen, alkyl, hydroxyalkyl, alkoxyalkyl, or optionally substituted phenylalkyl; R¹⁰, R¹², R¹⁴, and R¹⁶ are independently hydrogen, alkyl, optionally substituted phenylalkyl, alkoxy, hydroxyalkyl, haloalkyl, alkoxyalkyl, carboxyalkyl, cyanoalkyl, aminoalkyl, aminocarbonylalkyl, alkylaminocarbonylalkyl, dialkylaminocarbonylalkyl, or acyl or R⁴ and R⁵ together form methylenedioxy; and individual isomers, mixtures of isomers; or a pharmaceutically acceptable salt thereof and a pharmaceutically acceptable excipient in combination with radiation therapy and optionally in combination with one or more compound(s) independently selected from an estrogen receptor modulator, an androgen receptor modulator, retinoid receptor modulator, a cytotoxic agent, another antiproliferative agent, a prenyl-protein transferase inhibitor, an HMG-CoA reductase inhibitor, an HIV protease inhibitor, a reverse transcriptase inhibitor, or an angiogenesis inhibitor. Preferably, the combination therapy is carried out with a compound of Formula Ia, Ib, Ic, or Id.

Further aspects of this invention are disclosed in U.S. Provisional Application Ser. No. 60/355,700, filed on Feb. 7, 2002, the disclosure of which is incorporated herein by reference in its entirety.

### DETAILED DESCRIPTION OF THE INVENTION

Definitions:

Unless otherwise stated, the following terms used in the specification and claims are defined for the purposes of this Application and have the following meanings:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alkylene” means a linear saturated divalent hydrocarbon radical of one to six carbon atoms or a branched saturated divalent hydrocarbon radical of three to six carbon atoms e.g., methylene, ethylene, propylene, 1-methylpropylene, 2-methylpropylene, butylene, pentylene, and the like.

“Alkenyl” means a linear monovalent hydrocarbon radical of two to six carbon atoms or a branched monovalent hydrocarbon radical of three to six carbon atoms containing one or two double bonds, e.g., ethenyl, propenyl, 2-propenyl, butenyl (including all isomeric forms), and the like.

“Alkylthio” means a radical —SR where R is alkyl as defined above, e.g., methylthio, ethylthio, propylthio (including all isomeric forms), butylthio (including all isomeric forms), and the like.

“Amino” means a radical —NH₂, or an N-oxide derivative, or a protected derivative thereof such as —NH→O, —NHBoc, —NHCBz, and the like.

“Acyl” means a radical —COR where R is alkyl or trifluoromethyl, e.g., methylcarbonyl, trifluoromethylcarbonyl, and the like.

“Alkylamino” means a radical —NHR where R is alkyl as defined above, or an N-oxide derivative, or a protected derivative thereof, e.g., methylamino, ethylamino, n-, iso-propylamino, n-, iso-, tert-butylamino, methylamino-N-oxide, and the like.

“Alkoxy” means a radical —OR where R is alkyl as defined above, e.g., methoxy, ethoxy, propoxy, or 2-propoxy, n-, iso-, or tert-butoxy, and the like.

“Alkoxycarbonyl” means a radical —COOR where R is alkyl as defined above, e.g., methoxycarbonyl, ethoxycarbonyl, n-propoxycarbonyl, or 2-propoxycarbonyl, n-, iso-, or tert-butoxycarbonyl, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, 2-ethoxyethyl, and the like.

“Aminoalkyl” means a linear monovalent hydrocarbon radical of two to six carbon atoms or a branched monovalent hydrocarbon radical of three to six carbons substituted with at least one, preferably one or two, —NRR′ where R and R′ are independently selected from hydrogen, alkyl, or —COR^{a} where R^{a} is alkyl, or an N-oxide derivative, or a protected derivative thereof e.g., aminomethyl, methylaminoethyl, 2-ethylamino-2-methylethyl, 1,3-diaminopropyl, dimethylaminomethyl, diethylaminoethyl, acetylaminopropyl, and the like.

“Aryl” means a monovalent monocyclic or bicyclic aromatic hydrocarbon radical of 6 to 12 ring atoms e.g., phenyl, naphthyl or anthracenyl.

“Aminocarbonyl” means a radical —CONH₂ or a protected derivative thereof.

“Aminocarbonylalkyl” means a alkylene)-R where R is aminocarbonyl as defined above e.g., aminocarbonylmethyl, aminocarbonylethyl, aminocarbonylpropyl, and the like.

“Alkylaminocarbonylalkyl” means a alkylene)-COR where R is alkylamino group as defined above e.g., methylaminocarbonylmethyl, ethylaminocarbonylethyl, methylaminocarbonylpropyl, and the like.

“Cycloalkyl” means a cyclic saturated monovalent hydrocarbon radical of three to six carbon atoms, e.g., cyclopropyl, cyclobutyl, cyclopentyl, or cyclohexyl.

“Cycloalkylalkyl” means a alkylene)-R where R is cycloalkyl as defined above; e.g., cyclopropylmethyl, cyclobutylmethyl, cyclopentylethyl, or cyclohexylmethyl, and the like.

“Carboxyalkyl” means a radical -(alkylene)-COOH, e.g., carboxymethyl, carboxyethyl, 1-, 2-, or 3-carboxypropyl, and the like.

“Cyanoalkyl” means a radical alkylene)-CN, e.g., cyanomethyl, cyanoethyl, cyanopropyl, and the like.

“Dialkylamino” means a radical —NRR′ where R and R′ are independently alkyl as defined above, e.g., dimethylamino, diethylamino, methylpropylamino, methylethylamino, n-, iso-, or tert-butylamino, and the like.

“Dialkylaminocarbonylalkyl” means a -(alkylene)-COR where R is dialkylamino group as defined above e.g., dimethylaminocarbonylmethyl, metylethylaminocarbonylethyl, diethylaminocarbonylpropyl, and the like.

“Halo” means fluoro, chloro, bromo, and iodo, preferably fluoro or chloro.

“Haloalkyl” means alkyl substituted with one or more halogen atoms, preferably one to three halogen atoms, preferably fluorine or chlorine, including those substituted with different halogens, e.g., —CH₂Cl, —CF₃, —CHF₂, and the like.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, I-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Heterocycloalkyl” means a saturated monovalent cyclic group of 3 to 8 ring atoms in which one or two ring atoms are heteroatoms selected from N, O, or S(O)n, where n is an integer from 0 to 2, the remaining ring atoms being C. More specifically the term heterocycloalkyl; includes, but is not limited to, pyrrolidino, piperidino, morpholino, piperazino, tetrahydropyranyl, and thiomorpholino, and the derivatives thereof and N-oxide or a protected derivative thereof.

“Heterocycloamino or optionally substituted heterocycloamino” means a saturated monovalent cyclic group of 3 to 8 ring atoms in which one or two ring atoms are heteroatoms selected from N, O, or S(O)n, where n is an integer from 0 to 2, the remaining ring atoms being C, provided that at least one of the heteroatom is N. More specifically the term heterocycloamino; includes, but is not limited to, pyrrolidino, piperidino, morpholino, or piperazino, and the derivatives thereof and N-oxide or a protected derivative thereof. The heterocycloamino group is optionally substituted with one, two or three substituents independently selected from alkyl, halo, alkoxy, trifluoromethyl, trifluoromethoxy, amino, alkylamino, dialkylamino, hydroxy, cyano, nitro, optionally substituted phenylalkyl, optionally substituted heteroaralkyl, aminocarbonyl, hydroxyalkyl, alkoxycarbonyl, aminoalkyl, or carboxy. The optionally substituted heterocycloamino group is a subset of optionally substituted heterocycloalkyl.

“Heterocycloaminoalkyl” means a radical alkylene)-heterocycloamino. More specifically the term heterocycloaminoalkyl; includes, but is not limited to, pyrrolidin-1-ylmethyl, piperidin-1-ylmethyl, morpholin-4-methyl, and piperazin-1-methyl.

“Heteroaryl” means a monovalent monocyclic or bicyclic aromatic radical of 5 to 10 ring atoms containing one or more, preferably one or two ring heteroatoms selected from N, O, or S, the remaining ring atoms being carbon. More specifically the term heteroaryl includes, but is not limited to, pyridyl, pyrrolyl, imidazolyl, thienyl, furanyl, indolyl, quinolyl, pyrazine, pyrimidine, pyridazine, oxazole, isooxazolyl, benzoxazole, quinoline, isoquinoline, benzopyranyl, and thiazolyl, and the derivatives thereof, or N-oxide or a protected derivative thereof. When the heteroaryl ring is divalent it has been referred to as heteroarylene in this application. For example, when Ar¹ in the compounds of Formula I is a six membered heteroarylene ring containing one or two nitrogen ring atoms, the rest of the ring atoms being carbon it includes, but is not limited to rings such as: 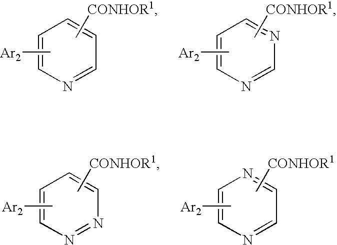
and the like.

“Methylenedioxy” means a radical O—CH₂—O—.

The present invention also includes the prodrugs of compounds of Formula I. The term prodrug is intended to represent covalently bonded carriers, which are capable of releasing the active ingredient of Formula I when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of Formula I include compounds wherein a hydroxy, amidino, guanidino, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula I), amides (e.g, trifluoroacetylamino, acetylamino, and the like), and the like. Prodrugs of compounds of Formula I are also within the scope of this invention.

The present invention also includes N-oxide derivatives and protected derivatives of compounds of Formula I. For example, when compounds of Formula I contain an oxidizable nitrogen atom, the nitrogen atom can be converted to an N-oxide by methods well known in the art. Also when compounds of Formula I contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. 1981, the disclosure of which is incorporated herein by reference in its entirety. The protected derivatives of compounds of Formula I can be prepared by methods well known in the art.

A “pharmaceutically acceptable salt” of a compound means a salt that is pharmaceutically acceptable and that possesses the desired pharmacological activity of the parent compound. Such salts include:
acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present invention may have asymmetric centers. Compounds of the present invention containing an asymmetrically substituted atom may be isolated in optically active or racemic forms. It is well known in the art how to prepare optically active forms, such as by resolution of materials. All chiral, diastereomeric, racemic forms are within the scope of this invention, unless the specific stereochemistry or isomeric form is specifically indicated.

Certain compounds of Formula I can exist as tautomers. All possible tautomers are within the scope of this invention. Additionally, as used herein the terms alkyl includes all the possible isomeric forms of said alkyl group albeit only a few examples are set forth. Furthermore, when the cyclic groups such as aryl, heteroaryl, heterocycloalkyl are substituted, they include all the positional isomers albeit only a few examples are set forth.

“Optionally substituted phenyl” means a phenyl ring optionally substituted with one, two, or three substituents independently selected from alkyl, halo, alkoxy, alkylthio, trifluoromethyl, trifluoromethoxy, amino, alkylamino, dialkylamino, hydroxy, cyano, nitro, methylenedioxy, aminocarbonyl, hydroxyalkyl, alkoxycarbonyl, aminoalkyl, or carboxy or optionally substituted with five fluorine atoms.

“Optionally substituted phenyloxy” means a radical —OR where R is optionally substituted phenyl as defined above e.g., phenoxy, chlorophenoxy, and the like.

“Optionally substituted phenylaminoalkyl” means a radical -alkylene-NHR where R is optionally substituted phenyl as defined above e.g., phenylaminomethyl, phenylaminoethyl, and the like.

“Optionally substituted phenylalkyl” means a radical alkylene)-R where R is optionally substituted phenyl as defined above e.g., benzyl, phenylethyl, and the like.

“Optionally substituted phenylalkenyl” means a radical alkenyl)-R where R is optionally substituted as defined above e.g., phenylethenyl, phenylpropenyl, and the like.

“Optionally substituted phenoxyalkyl” means a radical -(alkylene)-OR where R is optionally substituted phenyl as defined above e.g., phenoxymethyl, phenoxyethyl, and the like.

“Optionally substituted heteroaryl” means a heteroaryl ring as defined above which is optionally substituted with one, two, or three substituents independently selected from alkyl, halo, alkoxy, trifluoromethyl, trifluoromethoxy, amino, alkylamino, dialkylamino, hydroxy, cyano, nitro, aminocarbonyl, hydroxyalkyl, alkoxycarbonyl, aminoalkyl, optionally substituted phenyl, optionally substituted phenoxy, carboxy, or heteroaryl that is optionally substituted with alkyl, halo, hydroxy, alkoxy, carboxy, amino, alkylamino, or dialkylamino. More specifically the term optionally substituted heteroaryl includes, but is not limited to, pyridyl, pyrrolyl, imidazolyl, thienyl, furanyl, indolyl, quinolyl, pyrazine, pyrimidine, pyridazine, oxazole, isooxazolyl, benzoxazole, quinoline, isoquinoline, benzopyranyl, and thiazolyl, and the derivatives thereof, or N-oxide or a protected derivative thereof.

“Optionally substituted heteroaryloxyalkyl” means a -(alkylene)-OR where R is optionally substituted heteroaryl ring as defined above.

“Optionally substituted heteroaralkyl” means a -(alkylene)-R where R is optionally substituted heteroaryl ring as defined above.

“Optionally substituted heterocycloalkyl” means a heterocycloalkyl group as defined above which is optionally substituted with one, two, or three substituents independently selected from alkyl, halo, alkoxy, trifluoromethyl, trifluoromethoxy, amino, alkylamino, dialkylamino, hydroxy, cyano, nitro, optionally substituted phenylalkyl, optionally substituted heteroaralkyl aminocarbonyl, hydroxyalkyl, alkoxycarbonyl, aminoalkyl, or carboxy.

“Optionally substituted heterocycloalkylalkyl” means a alkylene)-R where R is optionally substituted heterocycloalkyl ring as defined above.

“Optional” or “optionally” means that the subsequently described event or circumstance may but need not occur, and that the description includes instances where the event or circumstance occurs and instances in which it does not. For example, “heterocycloalkyl group optionally mono- or di-substituted with an alkyl group” means that the alkyl may but need not be present, and the description includes situations where the heterocycloalkyl group is mono- or disubstituted with an alkyl group and situations where the heterocycloalkyl group is not substituted with the alkyl group.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease;
(2) inhibiting the disease, i.e., arresting or reducing the development of the disease or its clinical symptoms; or
(3) relieving the disease, i.e., causing regression of the disease or its clinical symptoms.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

A “therapeutically effective amount” means the amount of a compound of Formula I that, when administered to a mammal for treating a disease, is sufficient to effect such treatment for the disease. The “therapeutically effective amount” will vary depending on the compound, the disease and its severity and the age, weight, etc., of the mammal to be treated.

### Compound Numbering

The numbering of the compounds of this invention is illustrated below:

The Ar¹ is phenylene and Ar² is phenyl, are numbered as follows:

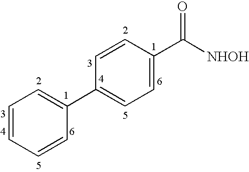

Representative compounds of Formula I are disclosed in Table I below:

| | |
|---|---|
| | I |
| 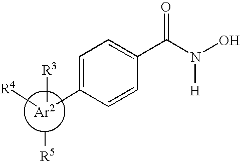 | |

| Cpd | |
|---|---|
| # | R³, R⁴, R⁵—Ar² |
| 1 | 3-(4-CH₃phenylCONH)-5-[N—(CH₃)—N—(OCH₃)NCO]-phenyl |
| 2 | 3-CH₃O-phenyl |
| 3 | 2-CH₃O-phenyl |
| 4 | 2,4-diCl-phenyl |
| 5 | 3,5-diCl-phenyl |
| 6 | 5-Cl-2-CH₃O-phenyl |
| 7 | 3-(CH₃)₂CH-phenyl |
| 8 | 3-NO₂-phenyl |
| 9 | 3-(CH₃SO₂NH)-phenyl |
| 10 | 3-NH₂-phenyl |
| 11 | 3-Cl-phenyl |
| 12 | 3-CF₃-phenyl |
| 13 | 2-CF₃-phenyl |
| 14 | 2,4-diF-phenyl |
| 15 | 2-F-phenyl |
| 16 | 2,4-diCH₃O-phenyl |
| 17 | 3,4-methylenedioxyphenyl |
| 18 | 3-(CH₃SO₂)-phenyl |
| 19 | 2,3-diCl-phenyl |
| 20 | 4-thiophen-3-ylphenyl |
| 21 | 3-CN-phenyl |
| 22 | 3-(phenylSO₂NH)-phenyl |
| 23 | 2-CH₃-phenyl |
| 24 | 2,3-diF-phenyl |
| 25 | 3,5-diCH₃-phenyl |
| 26 | 3-HO-phenyl |
| 27 | 3-(HOCH₂)-phenyl |
| 28 | 2,6-diF-phenyl |
| 29 | 2,5-diCH₃-phenyl |
| 30 | 2,3-diCH₃-phenyl |
| 31 | 2-Cl-phenyl |
| 32 | 4-(phenylSO₂NH)-phenyl |
| 33 | 3-(3-Cl-phenylSO₂NH)-phenyl |
| 34 | 3-(4-F-phenylSO₂NH)-phenyl |
| 35 | 3-(3,4-diClphenylSO₂NH)-phenyl |
| 36 | 3-(phenylCONH)-phenyl |
| 37 | 3-(benzylSO₂NH)-phenyl |
| 38 | 3-(4-ClphenylSO₂NH)-phenyl |
| 39 | 3-(3-CF₃phenylSO₂NH)-phenyl |
| 40 | 4,5-diphenyl-imidazol-2-yl |
| 41 | 4-benzimidazol-2-yl |
| 42 | 3-(4-CH₃O-phenylSO₂NH)-phenyl |
| 43 | 3-(3,4-diClphenylCONH)-phenyl |
| 44 | 1-CH₃benzimidazol-2-yl |
| 45 | 3-(3-CH₃OphenylNHCONH)-phenyl |
| 46 | 3-(3,4-diCH₃OphenylCONH)-phenyl |
| 47 | 3-(2,5-diCH₃OphenylSO₂NH)-phenyl |
| 48 | 3-(4-CF₃OphenylSO₂NH)-phenyl |
| 49 | 3-(3-F-phenylCONH)-phenyl |
| 50 | 3-(2,4-diCl-phenylCONH)-phenyl |
| 51 | 3-(phenylCONH)-phenyl |
| 52 | 3-(4-CH₃-phenylCONH)-phenyl |
| 53 | 3-(3-CF₃-phenylCONH)phenyl |
| 54 | 3-(phenylCONH)-5-(phenylNHCO)-phenyl |
| 55 | 3-(phenylSO₂NH)-5-(phenylNHCO)-phenyl |
| 56 | 3-benzyloxyphenyl |
| 57 | 3-(3-CH₃O-benzyloxy)-phenyl |
| 58 | 3-(3,4-methylenedioxyphenylCONH)-phenyl |
| 59 | 3-(N-3-methylbutylNHCO)-phenyl |
| 60 | 3-(N-methylNHCO)-phenyl |
| 61 | 3-(2-propylCONH)-phenyl |
| 62 | 5-fluoro-7-phenoxybenzimidazol-2-yl |
| 63 | 5-fluoro-7-(2-phenylethoxy)benzimidazol-2-yl |
| 64 | 5-fluoro-7-(4-CH₃Ophenoxy)benzimidazol-2-yl |
| 65 | 3-benzylCONH-phenyl |
| 66 | 3-(2-phenylethylCONH)-phenyl |
| 67 | 5-fluoro-7-tetrahydrofuran-2-ylmethyloxybenzimidazol-2-yl |
| 68 | 3-[2-(4-CF₃phenyl)ethylCONH]-phenyl |
| 69 | 3-[2-(4-CH₃Ophenyl)ethylCONH]-phenyl |
| 70 | 5-(benzylNHCO)-3-(phenylCONH)phenyl |
| 71 | 5-carboxy-3-(phenylCONH)-phenyl |
| 72 | 5-(N,N-dimethylNCO)-3-(phenylCONH)-phenyl |
| 73 | 5-(morpholin-4-ylCO)-3-(phenylCONH)-phenyl |
| 74 | 5-(N,N-dimethylNCO)-3-(4-CH₃-phenylCONH)-phenyl |
| 75 | 5-(morpholin-4-ylCO)-3-(4-CH₃-phenylCONH)-phenyl |
| 76 | 3-(4-CH₃phenylCONH)-5-(phenylNHCO)-phenyl |
| 77 | 3-(3,5-diCl-phenylCONH)phenyl |
| 78 | 3-(phenoxymethylCONH)phenyl |
| 79 | 5-fluoro-7-(2-CH₃-propoxy)benzimidazol-2-yl |
| 80 | 5-fluoro-7-cyclohexyloxybenzimidazol-2-yl |
| 81 | 3-(3-CH₃-butylCONH)phenyl |
| 82 | 3-(thiophen-2-ylmethylCONH)phenyl |
| 83 | 3-[2-(2,4-diCl-phenyl)ethylCONH]phenyl |
| 84 | 3-[2-(3,4-methylenedioxyphenyl)-ethylCONH]phenyl |
| 85 | 5-(N,N-dimethylNCO)-3-(4-CH₃O-phenylCONH)phenyl |
| 86 | 3-(4-CH₃O-phenylCONH)-5-(morpholin-4-ylCO)-phenyl |
| 87 | 3-(4-CH₃O-phenylCONH)-5-(phenylNHCO)-phenyl |
| 88 | 5-(N,N-dimethylNCO)-3-(3,4-di-CH₃O-phenylCONH)phenyl |
| 89 | 3-(3,4-di-CH₃O-phenylCONH)-5-(morpholin-4-ylCO)-phenyl |
| 90 | 3-(3,4-di-CH₃O-phenylCONH)-5-(phenylNHCO)-phenyl |
| 91 | 3-(4-CF₃-phenylCONH)phenyl |
| 92 | 3-(4-CH₃CH₂O-phenylCONH)phenyl |
| 93 | 3-(4-di-CH₃N-phenylCONH)phenyl |
| 94 | 3-(4-F-phenylCONH)phenyl |
| 95 | 3-(2,4-diF-phenylCONH)phenyl |
| 96 | 3-(4-Cl-phenylCONH)phenyl |
| 97 | 3-(N,N-dimethylNCO)phenyl |
| 98 | 3-(4-CH₃-phenylCONH)-5-(piperidin-1-ylCO)-phenyl |
| 99 | 3-(4-CH₃-phenylCONH)-5-(piperazin-1-ylCO)-phenyl |
| 100 | 5-(CH₃NHCO)-3-(4-CH₃-phenylCONH)phenyl |
| 101 | 5-(HOOCCH₂N(CH₃)CO)-3-(4-CH₃-phenylCONH)phenyl |
| 102 | 5-(NH₂COCH₂NHCO)-3-(4-CH₃-phenylCONH)phenyl |
| 103 | 5-{N—[CH₃NH(CH₂)₂]N(CH₃)CO}-3-(4-CH₃- |
| | phenylCONH)}phenyl |
| 104 | 5-(HOOCCH₂—NHCO)-3-(4-CH₃-phenylCONH)phenyl |
| 105 | 3-(4-isopropylphenylCONH)phenyl |
| 106 | 3-(4-CF₃O-phenylCONH)phenyl |
| 107 | 3-(3-F-4-CH₃O-phenylCONH)phenyl |
| 108 | 3-(4-CH₃O-2-CH₃-phenylCONH)phenyl |
| 109 | 3-(2,4-diCH₃O-phenylCONH)phenyl |
| 110 | 3-(4-Cl-2-CH₃O-phenylCONH)phenyl |
| 111 | 3-(pyridin-4-ylCONH)phenyl |
| 112 | 3-(pyridin-3-ylCONH)phenyl |
| 113 | 3-(morpholin-4-ylCO)phenyl |
| 114 | 3-[N—(CH₃)—N-(phenyl)NCO)phenyl |
| 115 | 3-(4-ClphenylNHCO)phenyl |
| 116 | 3-[2-(4-CH₃Ophenyl)ethylNHCO]phenyl |
| 117 | 5-(N,N-dimethylNCO]-3-(3,4-methylenedioxy- |
| | phenylCONH)phenyl |
| 118 | 3-(3,4-methylenedioxyphenylCONH)-5-(morpholin-4-ylCO)- |
| | phenyl |
| 119 | 3-(3,4-methylenedioxy-phenylCONH)-5-(phenylNHCO]-phenyl |
| 120 | 3-(2,4-diCl-phenylCONH)-5-[N,N-dimethylNCO]-phenyl |
| 121 | 3-(2,4-diCl-phenylCONH)-5-(phenylNHCO)phenyl |
| 122 | 5-(N,N-dimethylNCO)-3-(4-Cl-phenylCONH)phenyl |
| 123 | 5-(morpholin-4-ylCO)-3-(4-Cl-phenylCONH)phenyl |
| 124 | 3-(4-Cl-benzylNHCO)phenyl |
| 125 | 3-(2-phenylethylNHCO)phenyl |
| 126 | 3-(2-hydroxyethylNHCO)phenyl |
| 127 | 3-(piperidin-1-ylcarbonyl)phenyl |
| 128 | 5-[N—(CH₃)—N—(CH₃CH₂)NCO]-3-(4-CH₃- |
| | phenylCONH)phenyl |
| 129 | 3-(4-CH₃-phenylCONH)-5-(4-methylpiperazin-1-ylCO)-phenyl |
| 130 | 5-(3-NH₂CO-piperidin-1-ylCO)-3-(4-CH₃-phenylCONH)phenyl |
| 131 | 5-(N,N-diethylNCO)-3-(4-CH₃-phenylCONH)phenyl |
| 132 | 5-{2-(N-methylamino)ethylNHCO}-3-(4-CH₃- |
| | phenylCONH)phenyl |
| 133 | 5-(4-RS—HO-piperidin-1-ylCO)-3-(4-CH₃-phenylCONH)phenyl |
| 134 | 3-(4-CH₃-phenylCONH)-5-(pyrrolidin-1-ylCO)-phenyl |
| 135 | 5-[3-RS—(HOCH₂)-piperidin-1-ylCO]-3-(4-CH₃- |
| | phenylCONH)phenyl |
| 136 | 5-[2-RS—(HOCH₂)-piperidin-1-ylCO]-3-(4-CH₃- |
| | phenylCONH)phenyl |
| 137 | 5-[HO(CH₂)₂—N(CH₃)CO]-3-(4-CH₃-phenylCONH)phenyl |
| 138 | 5-(4-Cl-benzylNHCO)-3-(4-CH₃-phenylCONH)phenyl |
| 139 | 5-(3-methylbutylNHCO)-3-(4-CH₃-phenylCONH)phenyl |
| 140 | 3-(4-CH₃-phenylCONH)-5-{2-(phenyl)ethylNHCO}-phenyl |
| 141 | 5-{2-(4-CH₃O-phenyl)ethylNHCO}-3-(4-CH₃- |
| | phenylCONH)phenyl |
| 142 | 3-(2-CH₃-phenylCONH)-phenyl |
| 143 | 3-(2,4-diCH₃-phenylCONH)-phenyl |
| 144 | 3-(2,5-diCH₃-phenylCONH)-phenyl |
| 145 | 3-(2-CH₃-thiophen-5-ylCONH)-phenyl |
| 146 | 3-(4-CH₃-phenylCONH)-5-[N-(phenyl)-N—(CH₃)NCO]-phenyl |
| 147 | 3-[2-(4-CH₃Ophenyl)ethylNHCO]-phenyl |
| 148 | 3-(4-Cl-benzylNHCOCH₂)-phenyl |
| 149 | 5-(benzylNHCO)-3-(4-CH₃-phenylCONH)phenyl |
| 150 | 5-{HO(CH₂)₂NHCO}-3-(4-CH₃-phenylCONH)phenyl |
| 151 | 5-{[HO(CH₂)₂]₂NCO}-3-(4-CH₃-phenylCONH)phenyl |
| 152 | 3-[(CH₃)₂NCOCH₂]-phenyl |
| 153 | 3-[morpholin-4-ylCOCH₂]-phenyl |
| 154 | 5-[4-RS—HOCH₂piperidin-1-ylCO]-3-(4-CH₃- |
| | phenylCONH)phenyl |
| 155 | 5-[3-RS—CH₃CH₂OCOpiperidin-1-ylCO]-3-(4-CH₃- |
| | phenylCONH)phenyl |
| 156 | 3-[(N-(phenyl)-N—(CH₃)NCOCH₂]-phenyl |
| 157 | 3-(2-(phenyl)ethylNCOCH₂]-phenyl |
| 158 | 5-[4-RS—NH₂COpiperidin-1-ylCO]-3-(4-CH₃- |
| | phenylCONH)phenyl |
| 159 | 5-[2-RS—NH₂COpyrrolidin-1-ylCO]-3-(4-CH₃- |
| | phenylCONH)phenyl |
| 160 | 5-[4-RS—CH₃OCOpiperidin-1-ylCO]-3-(4-CH₃- |
| | phenylCONH)phenyl |
| 161 | 5-[NH(CH₃)COCH₂NHCO]-3-(4-CH₃-phenylCONH)phenyl |
| 162 | 3-{N(CH₃)₂COCH₂N(CH₃)CO}-3-(4-CH₃-phenylCONH)phenyl |
| 163 | 5-{NH₂COCH₂N(CH₃)CO}-3-(4-CH₃-phenylCONH)phenyl |
| 164 | 5-[(HOCH₂)₂CHNHCO]-3-(4-CH₃-phenylCONH)phenyl |
| 165 | 3-(2,4-diCl-phenylCONH)-5-(pyrrolidin-1-ylCO)-phenyl |
| 166 | 3-(4-Cl-phenylNHCOCH₂)phenyl |
| 167 | 3-(2,4-diCl-phenylCONH)-5-(piperidin-1-ylCO)-phenyl |
| 168 | 3-(2,4-diCl-phenylCONH)-5-[2-(S)—HOCH₂pyrrolidin-1-yl-CO]- |
| | phenyl |
| 169 | 3-(2,4-diCl-phenylCONH)-5-[2-(R)-HOCH₂pyrrolidin-1-ylCO]- |
| | phenyl |
| 171 | 4-benzyloxybenzimidazol-2-yl |
| 172 | 3-(4-CH₃OphenylCONH)phenyl |
| 173 | 3-(benzylNHCO)phenyl |
| 174 | 3-(CF₃CH₂NHCO)phenyl |
| 175 | 3-(2,4-dichlorophenylCONH)-5-(morpholin-4-ylCO)phenyl |
| 176 | 3-(2,4-diClphenylCONH)-5-[morpholin-4-ylCO]phenyl |
| 177 | 3-(4-CH₃phenylCONH)-5-[N—(OCH₃)—N—(CH₃)—NCO]phenyl |
| 178 | 3-(4-CH₃phenylCONH)-5-(4-Cl-phenylNHCO)phenyl |
| 179 | 3-(benzylCONH)-5-(pyrrolidin-1-ylCO)phenyl |
| 180 | 3-(2-phenylethylCONH)-5-(pyrrolidin-1-ylCO)phenyl |
| 181 | 3-(4-OCH₃-2-CH₃phenylCONH)-5-(pyrrolidin-1-ylCO)phenyl |
| 182 | 3-(4-OCH₃-2-CH₃phenylCONH)-5-(piperidin-1-ylCO)phenyl |
| 183 | 3-(phenylCH═CHCONH)-5-(pyrrolidin-1-ylCO)phenyl |
| 184 | 3-(2,4-diCH₃phenylCONH)-5-(pyrrolidin-1-ylCO)phenyl |
| 185 | 3-(2,4-diCH₃phenylCONH)-5-(piperidin-1-ylCO)phenyl |
| 186 | 3-(2,4-diCH₃phenylCONH)-5-[(C₂H₅)₂NCO]phenyl |
| 187 | 3-(4-OCH₃-2-CH₃phenylCONH)-5-[(C₂H₅)₂NCO]phenyl |
| 188 | 3-(2,4-diClphenylCONH)-5-[(CH₃)₂NCH₂]phenyl |
| 189 | 3-(4-CH₃phenylCONH)-5-[(CH₃)₂NCH₂]phenyl |
| 190 | 3-(phenylSO₂NH)-5-[C₃H₇OCH₂]phenyl |
| 191 | 3-(2,4-diClphenylCONH)-(piperidin-1-ylCH₂)phenyl |
| 192 | 3-(4-OCH₃phenylCONH)-5-[C₃H₇OCH₂]phenyl |
| 193 | 3-(2-propylCONH)-5-[C₃H₇OCH₂]phenyl |
| 194 | 3-(phenylNHCONH)-5-[C₃H₇OCH₂]phenyl |
| 195 | 3-(benzylNH)-5-[C₃H₇OCH₂]phenyl |
| 196 | 3-(phenylCH═CHCONH)-5-[C₃H₇OCH₂]phenyl |
| 197 | 3-(phenylNHCH₂CONH)-5-[C₃H₇OCH₂]phenyl |
| 198 | 3-(4-OCH₃phenylCONH)-5-[benzylOCH₂]phenyl |
| 199 | 3-(4-OCH₃phenylCONH)-5-[phenylOCH₂]phenyl |
| 200 | 3-(4-OCH₃phenylCONH)-5-[phenylCH═CHCH₂OCH₂]phenyl |
| 201 | 3-(2,4-diClphenylCONH)-5-(pyrrolidin-1-ylCH₂)phenyl |
| 202 | 3-(indol-3-ylmethylCONH)-5-[(CH₃)₂NCO]phenyl |
| 203 | 3-(2,4-diClbenzylNH)-5-[(CH₃)₂NCO]phenyl |
| 204 | 3-(pyridin-4-ylmethylCONH)-5-[(CH₃)₂NCO]phenyl |
| 205 | 3-(2,4-diClphenylCONH)-5[N—(—CH₂CH₂OH)—N—(CH₃) |
| | NCO]phenyl |
| 206 | 3-(furan-3-ylCONH)-5-[(CH₃)₂NCH₂]phenyl |
| 207 | 3-(phenylNHCONH)phenyl |
| 208 | 3-(4-OCH₃phenylCONH)phenyl |
| 209 | 3-(benzylNH)phenyl |
| 210 | 3-(4-OCH₃benzylNH)phenyl |
| 211 | 3-(4-CH₃benzylNH)phenyl |
| 213 | 3-(phenylCONHCH₂)phenyl |
| 214 | 3-(4-ClphenylNHCOCH₂)phenyl |
| 215 | 3-(pyridin-3-ylmethylCONHCH₂)phenyl |
| 216 | 3-[N-phenylCON—(—CH₂CH₂OH)NCH₂]phenyl |
| 217 | 3-[N-benzyl-N—(—CH₂CH₂OH)NCH₂]phenyl |
| 218 | 3-[N-benzyl-N—(—CH₂CH₂OCH₃)NCH₂]phenyl |
| 219 | 3-(benzylNCH₂)phenyl |
| 220 | 3-(2-indol-3-ylethylNCH₂)phenyl |
| 221 | 3-(3,4-methylenedioxybenzylNCH₂)phenyl |
| 222 | 3-(pyridin-4-ylmethylNCH₂)phenyl |
| 223 | 3-(pyridin-3-yl-OCH₂)phenyl |
| 224 | 3-(2-pyridin-3-ylethylNCH₂)phenyl |
| 225 | 3-(phenyl-OCH₂)phenyl |
| 226 | 3-(4-CH₃phenyl-OCH₂)phenyl |
| 227 | 3-(4-Clphenyl-OCH₂)phenyl |
| 228 | 3-(3-phenylpropylNHCH₂)phenyl |
| 229 | 3-(phenylNHCH₂)phenyl |
| 230 | 3-(4-CH₃phenylNHCH₂)phenyl |
| 231 | 3-(4-ClphenylNHCH₂)phenyl |
| 232 | 3-(2-(4-OCH₃phenylethylNHCOCH₂)phenyl |

and are named as follows:
N-hydroxy-4-(3-methoxyphenyl)benzamide;
N-hydroxy-4-(2-methoxyphenyl)benzamide;
N-hydroxy-4-(2,4-dichlorophenyl)benzamide;
N-hydroxy-4-(3,5-dichlorophenyl)benzamide;
N-hydroxy-4-(5-chloro-2-methoxyphenyl)benzamide;
N-hydroxy-4-(3-isopropylphenyl)benzamide;
N-hydroxy-4-(3-nitrophenyl)benzamide;
N-hydroxy-4-(3-methylsulfonylaminophenyl)benzamide;
N-hydroxy-4-(3-aminophenyl)benzamide;
N-hydroxy-4-(3-chlorophenyl)benzamide;
N-hydroxy-4-(3-trifluoromethylphenyl)benzamide;
N-hydroxy-4-(2-trifluoromethylphenyl)benzamide;
N-hydroxy-4-(2,4-difluorophenyl)benzamide;
N-hydroxy-4-(2-fluorophenyl)benzamide;
N-hydroxy-4-(2,4-dimethoxyphenyl)benzamide;
N-hydroxy-4-(3,4-methylenedioxyphenyl)benzamide;
N-hydroxy-4-(3-methylsulfonylphenyl)benzamide;
N-hydroxy-4-(2,3-dichlorophenyl)benzamide;
N-hydroxy-4-(4-thiophen-3-ylphenyl)benzamide;
N-hydroxy-4-(3-cyanophenyl)benzamide;
N-hydroxy-4-(3-phenylsulfonylaminophenyl)benzamide;
N-hydroxy-4-(2-methylphenyl)benzamide;
N-hydroxy-4-(2,3-difluorophenyl)benzamide;
N-hydroxy-4-(3,5-dimethylphenyl)benzamide;
N-hydroxy-4-(3-hydroxyphenyl)benzamide;
N-hydroxy-4-(3-hydroxymethylphenyl)benzamide;
N-hydroxy-4-(2,6-difluorophenyl)benzamide;
N-hydroxy-4-(2,5-dimethylphenyl)benzamide;
N-hydroxy-4-(2,3-dimethylphenyl)benzamide;
N-hydroxy-4-(2-chlorophenyl)benzamide;
N-hydroxy-4-(4-phenylsulfonylaminophenyl)benzamide;
N-hydroxy-4-[3-(3-chlorophenylsulfonylamino)phenyl]benzamide;
N-hydroxy-4-[3-(4-fluorophenylsulfonylamino)phenyl]benzamide;
N-hydroxy-4-[3-(3,4-dichlorophenylsulfonylamino)phenyl]benzamide;
N-hydroxy-4-[3-(phenylcarbonylamino)phenyl]benzamide;
N-hydroxy-4-[3-(benzylsulfonylamino)phenyl]benzamide;
N-hydroxy-4-[3-(4-chlorophenylsulfonylamino)phenyl]benzamide;
N-hydroxy-4-[3-(3-trifluoromethylphenylsulfonylamino)phenyl]benzamide;
N-hydroxy-4-(2,4-diphenylbenzimidazol-2-yl)benzamide;
N-hydroxy-4-(benzimidazol-2-yl)benzamide;
N-hydroxy-4-[3-(4-methoxyphenylsulfonylamino)phenyl]benzamide;
N-hydroxy-4-[3-(3,4-dichlorophenylcarbonylamino)phenyl]benzamide;
N-hydroxy-4-(1-methylbenzimidazol-2-yl)benzamide;
N-hydroxy-4-[3-(3-methoxyphenylureido)phenyl]benzamide;
N-hydroxy-4-[3-(3,4-dimethoxyphenylcarbonylamino)phenyl]benzamide;
N-hydroxy-4-[3-(2,5-dimethoxyphenylsulfonylamino)phenyl]benzamide;
N-hydroxy-4-[3-(4-trifluoromethoxyphenylsulfonylamino)phenyl]benzamide;
N-hydroxy-4-[3-(3-fluorophenylcarbonylamino)phenyl]benzamide;
N-hydroxy-4-[3-(2,4-dichlorophenylcarbonylamino)phenyl]benzamide;
N-hydroxy-4-[3-(phenylcarbonylamino)phenyl]benzamide;
N-hydroxy-4-[3-(4-methylphenylcarbonylamino)phenyl]benzamide;
N-hydroxy-4-[3-(3-trifluoromethylphenylcarbonylamino)phenyl]benzamide;
N-hydroxy-4-[5-(phenylaminocarbonyl)-3-(phenylcarbonylamino)phenyl]benzamide;
N-hydroxy-4-[5-(phenylaminocarbonyl)-3-(phenylsulfonylamino)phenyl]benzamide;
N-hydroxy-4-(3-benzyloxyphenyl)benzamide;
N-hydroxy-4-[3-(3-methoxybenzyloxy)phenyl]benzamide;
N-hydroxy-4-[3-(3,4-methylenedioxyphenylcarbonylamino)phenyl]benzamide;
N-hydroxy-4-[3-(N-3-methylbutylaminocarbonyl)phenyl]benzamide;
N-hydroxy-4-[3-(N-methylaminocarbonyl)phenyl]benzamide;
N-hydroxy-4-[3-(2-propylcarbonylamino)phenyl]benzamide;
N-hydroxy-4-(5-fluoro-7-phenoxybenzimidazol-2-yl)benzamide;
N-hydroxy-4-[5-fluoro-7-(2-phenylethoxy)benzimidazol-2-yl]benzamide;
N-hydroxy-4-[5-fluoro-7-(2-methoxyphenoxy)benzimidazol-2-yl]benzamide;
N-hydroxy-4-[3-(benzylcarbonylamino)phenyl]benzamide;
N-hydroxy-4-[3-(2-phenylethylcarbonylamino)phenyl]benzamide;
N-hydroxy-4-[5-fluoro-7-(tetrahydroyfuran-2-ylmethoxyoxy)benzimidazol-2-yl]benzamide;
N-hydroxy-4-{3-[2-(4-trifluorophenyl)ethylcarbonylamino]phenyl}benzamide;
N-hydroxy-4-{3-[2-(4-methoxyphenyl)ethylcarbonylamino]phenyl}benzamide;
N-hydroxy-4-[5-(N-benzylaminocarbonyt)-3-(phenylcarbonylamino)phenyl]benzamide;
N-hydroxy-4-[5-carboxy-3-(phenylcarbonylamino)phenyl]benzamide;
N-hydroxy-4-[5-(N,N-dimethylaminocarbonyl)-3-(phenylcarbonylamino)phenyl]benzamide;
N-hydroxy-4-[5-(morpholin-4-ylcarbonyl)-3-(phenylcarbonylamino)phenyl]benzamide;
N-hydroxy-4-[5-(N,N-dimethylaminocarbonyl)-3-(4-methylphenylcarbonylamino)phenyl]-benzamide;
N-hydroxy-4-[3-(4-methylphenylcarbonylamino)-5-(N-phenylaminocarbonyl)phenyl]-benzamide;
N-hydroxy-4-[3-(3,5-dichlorophenylcarbonylamino)phenyl]benzamide;
N-hydroxy-4-[3-(phenoxymethylcarbonylainino)phenyl]benzamide;
N-hydroxy-4-[5-fluoro-7-(2-methylpropoxy)benzimidazol-2-yl]benzamide;
N-hydroxy-4-[5-fluoro-7-(cyclohexyloxy)benzimidazol-2-yl]benzamide;
N-hydroxy-4-[3-(3-methylbutylcarbonylamino)phenyl]benzamide;
N-hydroxy-4-[3-(thiophen-2-ylmethylcarbonylamino)phenyl]benzamide;
N-hydroxy-4-{3-[2-(2,4-dichlorophenyl)ethylcarbonylamino]phenyl}benzamide;
N-hydroxy-4-{3-[2-(3,4-methylenedioxyphenyl)ethylcarbonylamino]phenyl}benzamide;
N-hydroxy-4-[5-(N,N-dimethylaminocarbonyl)-3-(4-methoxyphenylcarbonylamino)phenyl]-benzamide;
N-hydroxy-4-[3-(4-methoxyphenylcarbonylamno)-5-(morpholin-4-ylcarbonyl)phenyl]-benzamide;
N-hydroxy-4-[3-(4-methoxyphenylcarbonylamino)-5-(phenylaminocarbonyl)-phenyl]benzamide;
N-hydroxy-4-[5-(N,N-dimethylaminocarbonyl)-3-(3,4 methoxyphenylcarbonylanno)-phenyl]-benzamide;
N-hydroxy-4-[3-(3,4-dimethoxyphenylcarbonylamino)-5-(morpholin-4-ylcarbonyl)phenyl]-benzamide;
N-hydroxy-4-[3-(3,4-dimethoxyphenylcarbonylamino)-5-(phenylaminocarbonyl)phenyl]-benzamide;
N-hydroxy-4-[3-(4-trifluorophenylcarbonylamino)phenyl]benzamide;
N-hydroxy-4-[3-(4-ethoxyphenylcarbonylamino)phenyl]benzaxhide;
N-hydroxy-4-[3-(4-N,N-dimethylaminophenylcarbonylamino)phenyl]benzamide;
N-hydroxy-4-[3-(4-fluorophenylcarbonylamino)phenyl]benzamide;
N-hydroxy-4-[3-(2,4-difluorophenylcarbonylamino)phenyl]benzamide;
N-hydroxy-4-[3-(4-chlorophenylcarbonylamino)phenyl]benzamide;
N-hydroxy-4-[3-(N,N-dimethylaminocarbonyl)phenyl]benzamide;
N-hydroxy-4-[3-(4-methylphenylcarbonylamino)-5-(piperidin-1-ylcarbonyl)phenyl]-benzamide;
N-hydroxy-4-[3-(4-methylphenylcarbonylamino)-5-(piperazin-1-ylcarbonyl)phenyl]-benzamide;
N-hydroxy-4-[5-(N-methylaminocarbonyl)-3-(4-methylphenylcarbonylamino)phenyl]-benzamide;
N-hydroxy-4-[5-(N-carboxymethyl-N-methylaminocarbonyl)-3-(4-methylphenylcarbonyl-amino)phenyl]benzamide;
N-hydroxy-4-[5-(N-aminocarbonylmethylaminocarbonyl)-3-(4-methylphenylcarbonyl-amino)phenyl]-benzamide;
N-hydroxy-4-{5-[N-(2-N-methylaminoethyl)-N-(methyl)aminocarbonyl]-3-(4-methyl-phenylcarbonylamino)phenyl]-benzamide;
N-hydroxy-4-[5-(N-carboxymethylaminocarbonyl)-3-(4-methylphenylcarbonyl-amino)phenyl]-benzamide;
N-hydroxy-4-[3-(4-isopropylphenylcarbonylamino)phenyl]benzamide;
N-hydroxy-4-[3-(4-trifluoromethoxyphenylcarbonylamino)phenyl]benzamide;
N-hydroxy-4-[3-(3-fluoro-4-methoxyphenylcarbonylamino)phenyl]benzamide;
N-hydroxy-4-[3-(4-methoxy-2-methylphenylcarbonylamino)phenyl]benzamide;
N-hydroxy-4-[3-(2,4-dimethoxyphenylcarbonylamino)phenyl]benzamide;
N-hydroxy-4-[3-(4-chloro-2-methoxyphenylcarbonylamino)phenyl]benzamide;
N-hydroxy-4-[3-(pyridin-4-ylcarbonylamino)phenyl]benzamide;
N-hydroxy-4-[3-(pyridin-3-ylcarbonylamino)phenyl]benzamide;
N-hydroxy-4-[3-(morpholin-4-ylcarbonylamino)phenyl]benzamide;
N-hydroxy-4-[3-(N-methyl-N-phenylaminocarbonyl)phenyl]benzamide;
N-hydroxy-4-[3-(4-chlorophenylaminocarbonyl)phenyl]benzamide;
N-hydroxy-4-(3-[N-2-(4-methoxyphenyl)ethylaminocarbonyl]phenyl}benzamide;
N-hydroxy-4-[5-(N,N-dimethylaminocarbonyl)-3-(3,4-methylenedioxyphenylcarbonyl-amino)phenyl]benzamide;
N-hydroxy-4-[3-(3,4-methylenedioxyphenylcarbonylamino)-5-(morpholin-4-ylcarbonyl)-phenyl]benzamide;
N-hydroxy-4-[3-(3,4-methylenedioxyphenylcarbonylamino)-5-(N-phenylaminocarbonyl)-phenyl]benzamide;
N-hydroxy-4-[3-(2,4-dichlorophenylcarbonylamino)-5-(NN-dimethylaminocarbonyl)-phenyl]benzamide;
N-hydroxy-4-[3-(2,4-dichlorophenylcarbonylamino)-5-(N-phenylaminocarbonyl)phenyl]-benzamide;
N-hydroxy-4-[5-(N,N-dimethylaminocarbonyl)-3-(4-chlorophenylcarbonylamino)-phenyl]benzamide;
N-hydroxy-4-[5-(morpholin-4-ylcarbonyl)-3-(4-chlorophenylcarbonylamino)-phenyl]benzamide;
N-hydroxy-4-[3-(N-4-chlorobenzylaminocarbonyl)phenyl]benzamide;
N-hydroxy-4-[3-(N-2-phenylethylaminocarbonyl)phenyl]benzamide;
N-hydroxy-4-[3-(N-2-hydroxyethylaminocarbonyl)phenyl]benzamide;
N-hydroxy-4-[3-(piperidin-1-ylcarbonyl)phenyl]benzamide;
N-hydroxy-4-[5-(N-methyl-N-ethylaminocarbonyl)-3-(4-methylphenylcarbonylamino)-phenyl]benzamide;
N-hydroxy-4-[3-(4-methylphenylcarbonylamino)-5-(4-methylpiperazin-1-ylcarbonyl)phenyl]-benzamide;
N-hydroxy-4-[5-(3-(RS)-aminocarbonylpiperidin-1-ylcarbonyt)-3-(4-methylphenylcarbonyl-amino) phenyl)benzamide;
N-hydroxy-4-[5-(N,N-diethylaminocarbonyl)-3-(4-methylphenylcarbonylamino)-phenyl]benzamide;
N-hydroxy-4-{5-[N-2-(N-methylamino)ethylaminocarbonyl]-3-(4-methylphenyl-carbonylamino)-phenyl}-benzamide;
N-hydroxy-4-[5-(4-RS-hydroxypiperidin-1-ylcarbonyl)-3-(4-methylphenylcarbonylamino)-phenyl]benzamide;
N-hydroxy-4-[3-(4-methylphenylcarbonylamino)-5-(pyrrolidin-1-ylcarbonyl)phenyl]-benzamide;
N-hydroxy-4-[5-(3-(RS)-hydroxymethylpiperidin-1-ylcarbonyl)-3-(4-methylphenylcarbonyl-amino)phenyl]benzamide;
N-hydroxy-4-[5-(2-(RS)-hydroxymethylpiperidin-1-ylcarbonyl)-3-(4-methylphenylcarbonyl-amino)phenyl]benzamide;
N-hydroxy-4-{5-[N-(2-hydroxyethyl)-N-methylaminocarbonyl]-3-(4-methylphenylcarbonyl-amino) phenyl}benzamide;
N-hydroxy-4-[5-(N-4-chlorobenzylaminocarbonyl)-3-(4-methylphenylcarbonylamino)-phenyl]benzamide;
N-hydroxy-4-[5-(N-3-methylbutylaminocarbonyl)-3-(4-methylphenylcarbonylamino)-phenyl]benzamide;
N-hydroxy-4-[3-(4-methylphenylcarbonylamino)-5-(N-2-phenylethylaminocarbonyl)phenyl]-benzamide;
N-hydroxy-4-{5-[N-2-(4-methoxyphenyl)ethylaminocarbonyl]-3-(4-methylphenylcarbonyl-amino)phenyl]benzamide;
N-hydroxy-4-[3-(2-methylphenylcarbonylamino)phenyl]benzamide;
N-hydroxy-4-[3-(2,4-diinethylphenylcarbonylamino)phenyl]benzamide;
N-hydroxy-4-[3-(2,5-dimethylphenylcarbonylamino)phenyl]benzamide;
N-hydroxy-4-[3-(2-methylthiophen-5-ylcarbonylamino)phenyl]benzamide;
N-hydroxy-4-[3-(4-methylphenylcarbonylamino)-5-(N-phenyl-N-methylaminocarbonyl)phenyl]-benzamide;
N-hydroxy-4-{3-[N-2-(4-methoxyphenyl)ethylaminocarbonyl]phenyl}benzamide;
N-hydroxy-4-[3-(N-4-chlorobenzylaminocarbonylmethyl)phenyl]benzamide;
N-hydroxy-4-[5-(N-benzylaminocarbonyl)-3-(4-methylphenylcarbonyl-amino)phenyl]benzamide;
N-hydroxy-4-{5-[N-(2-hydroxyethyl)aminocarbonyl]-3-(4-methylphenylcarbonyl-amino)phenyl}benzamide;
N-hydroxy-4-(5-[N,N-bis(2-hydroxyethyl)aminocarbonyl]-3-(4-methylphenyl-carbonylamino)-phenyl]benzamide;
N-hydroxy-4-[3-(N,N-dimethylaminocarbonylmethyl)phenyl]benzamide;
N-hydroxy-4-[3-(morpholin-4-ylcarbonylmethyl)phenyl]benzamide;
N-hydroxy-4-[5-(4-(RS)-hydroxymethylpiperidin-1-ylcarbonyl)-3-(4-methylphenylcarbonyl-amino)phenyl]benzamide;
N-hydroxy-4-[5-(3-(RS)-ethoxycarbonylpiperidin-1-ylcarbonyl)-3-(4-methylphenylcarbonyl-amino)phenyl]benzamide;
N-hydroxy-4-[3-(N-phenyl-N-methylaminocarbonylmethyl)phenyl]benzamide;
N-hydroxy-4-[3-(N-2-phenylethylaminocarbonylmethyl)phenyl]benzamide;
N-hydroxy-4-[5-(4-(RS)-aminocarbonylpiperidin-1-ylcarbonyl)-3-(4-methylphenylcarbonyl-amino)phenyl]benzamide;
N-hydroxy-4-[5-(2-(RS)-aminocarbonylpyffolidin-1-ylcarbonyl)-3-(4-methylphenylcarbonyl-amino)phenyl]benzamide;
N-hydroxy-4-[5-(4-(RS)-ethoxycarbonylpiperidin-1-ylcarbonyl)-3-(4-methylphenylcarbonyl-amino)phenyl]benzamide;
N-hydroxy-4-{5-[N-(N-methylaminocarbonylmethyl)aminocarbonyl]-3-(4-methylphenyl-carbonyl-amino)phenyl}benzamide;
N-hydroxy-4-{5-[N-(N-dimethylaminocarbonylmethyl)-N-methylaminocarbonyl]-3-(4-methylphenylcarbonyl-amino)phenyl}benzamide;
N-hydroxy-4-(5-[N-(aminocarbonylmethyl)-N-methylaminocarbonyl]-3-(4-methylphenyl-carbonyl-amino)phenyl}benzamide;
N-hydroxy-4-{5-[N-(2-hydroxy-1-hydroxymethyl)ethylaminocarbonyl]-3-(4-methylphenyl-carbonyl-amino)phenyl}benzamide;
N-hydroxy-4-[3-(2,4-dichlorophenylcarbonylamino)-5-(pyrrolidin-1-ylcarbonyl)phenyl]-benzamide;
N-hydroxy-4-[3-(N-4-chlorophenylaminocarbonylmethyl)phenyl]benzamide;
N-hydroxy-4-[3-(2,4-dichlorophenylcarbonylamino)-5-(piperidin-1-ylcarbonyl)phenyl]-benzamide;
N-hydroxy-4-[3-(2,4-dichlorophenylcarbonylamino)-5-(2-(S)-hydroxymethylpyrrolidin-1-ylcarbonyl)phenyl]-benzamide;
N-hydroxy-4-[3-(2,4-dichlorophenylcarbonylamino)-5-(2-(R)-hydroxymethylpyrrolidin-1-ylcarbonyl)phenyl]-benzamide;
N-hydroxy-4-(4-benzyloxybenzimidazol-2-yl)benzamide;
N-hydroxy-4-[3-(4-methoxyphenylcarbonylamino)phenyl]benzamide;
N-hydroxy-4-[3-(4-N-benzylaminocarbonyl)phenyl]benzamide;
N-hydroxy-4-[3-(N-2,2,2-trifluoroethylaminocarbonyl)phenyl]benzamide;
N-hydroxy-4-[3-(2,4-dichlorophenylcarbonylamino)-5-(morpholin-4-ylcarbonyl)phenyl]-benzamide;
N-hydroxy-4-[3-(4-methylphenylcarbonylamino)-5-(N-methoxy-N-methylamino-carbonyl)phenyl]-benzamide;
N-hydroxy-4-[3-(4-methylphenylcarbonylamino)-5-(4-chlorophenylaminocarbonyl)phenyl]-benzamide;
N-hydroxy-4-[3-(benzylcarbonylamino)-5-(pyrrolidin-1-ylcarbonyl)phenyl]-benzamide;
N-hydroxy-4-[3-(2-phenylethylcarbonylamino)-5-(pyrrolidin-1-ylcarbonyl)phenyl]-benzamide;
N-hydroxy-4-[3-(4-methoxy-2-methylphenylcarbonylamino)-5-(pyrrolidin-1-ylcarbonyl)-phenyl]-benzamide;
N-hydroxy-4-[3-(4-methoxy-2-methylphenylcarbonylamino)-5-(piperidin-1-ylcarbonyl)-phenyl]-benzamide;
N-hydroxy-4-[3-(2-phenylethenylenecarbonylamino)-5-(pyrrolidin-1-ylcarbonyl)phenyl]-benzamide;
N-hydroxy-4-[3-(2,4-dimethylphenylcarbonylamino)-5-(pyrrolidin-1-ylcarbonyl)phenyl]-benzamide;
N-hydroxy-4-[3-(2,4-dimethylphenylcarbonylamino)-5-(piperidin-1-ylcarbonyl)phenyl]-benzamide;
N-hydroxy-4-[3-(2,4-dimethylphenylcarbonylamino)-5-(diethylaminocarbonyl)phenyl]-benzamide;
N-hydroxy-4-[3-(4-methoxy-2-methylphenylcarbonylamino)-5-(diethylaminocarbonyl)-phenyl]-benzamide;
N-hydroxy-4-[3-(2,4-dichlorophenylcarbonylamino)-5-(dimethylaminomethyl)phenyl]-benzamide;
N-hydroxy-4-[3-(4-methylphenylcarbonylamino)-5-(dimethylaminomethyl)phenyl]-benzamide;
N-hydroxy-4-[3-(phenylsulfonylamino)-5-(propoxymethyl)phenyl]-benzamide;
N-hydroxy-4-[3-(2,4-dichlorophenylcarbonylamino)-5-(piperidin-1-ylmethyl)phenyl]-benzamide;
N-hydroxy-4-[3-(4-methoxyphenylcarbonylamino)-5-(propoxymethyl)phenyl]-benzamide;
N-hydroxy-4-[3-(2-propylcarbonylamino)-5-(propoxymethyl)phenyl]-benzamide;
N-hydroxy-4-[3-(3-phenylureido)-S-(propoxymethyl)phenyl]-benzamide;
N-hydroxy-4-[3-(benzylamino)-5-(propoxymethyl)phenyl]-benzamide;
N-hydroxy-4-[3-(phenylethenylcarbonylamino)-5-(propoxymethyl)phenyl]-benzamide;
N-hydroxy-4-[3-(phenylaminomethylcarbonylamino)-5-(propoxymethyl)phenyl]-benzamide;
N-hydroxy-4-[3-(4-methoxyphenylcarbonylamino)-5-(benzyloxymethyl)phenyl]-benzamide;
N-hydroxy-4-[3-(4-methoxyphenylcarbonylamino)-5-(phenoxymethyl)phenyl]-benzamide;
N-hydroxy-4-[3-(4-methoxyphenylcarbonyliamino)-5-(phenylethenylmethyloxy-methyl)phenyl]-benzamide;
N-hydroxy-4-[3-(2,4-dichlorophenylcarbonylamino)-5-(pyrrolidin-1-ylmethyl)phenyl]-benzamide;
N-hydroxy-4-[3-(indol-3-ylmethylcarbonylamino)-5-(dimethylaminocarbonyl)phenyl]-benzamide;
N-hydroxy-4-[3-(2,4-dichlorobenzylamino)-5-(dimethylaminocarbonyl)phenyl]-benzade;
N-hydroxy-4-[3-(pyridin-4-ylmethylcarbonylamino)-5-(dimethylaminocarbonyl)phenyl]-benzamide;
N-hydroxy-4-[3-(2,4-dichlorophenylcarbonylamino)-5-[N-(2-hydroxyethyl)-N-methylaminocarbonyl)phenyl]-benzamide;
N-hydroxy-4-[3-(furan-3-ylcarbonylamino)-5-(dimethylaminomethyl)phenyl]-benzamide;
N-hydroxy-4-[3-(3-phenylureido)phenyl]benzamide;
N-hydroxy-4-[3-(4-methoxyphenylcarbonylamino)phenyl]benzamide;
N-hydroxy-4-[3-(benzylamino)phenyl]benzamide;
N-hydroxy-4-[3-(4-methoxybenzylamino)phenyl]benzamide;
N-hydroxy-4-[3-(4-methylbenzylamino)phenyl]benzamide;
N-hydroxy-4-[3-(phenylcarbonylaminomethyl)phenyl]benzamide;
N-hydroxy-4-[3-(4-chlorophenylaminocarbonylmethyl)phenyl]benzamide;
N-hydroxy-4-[3-(pyridin-3-ylmethylcarbonylaminomethyl)phenyl]benzamide;
N-hydroxy-4-{3-[N-(benzoyl)-N-(2-hydroxyethyl)aminomethyl]phenyl}benzamide;
N-hydroxy-4-{3-[N-(benzyl)-N-(2-hydroxyethyl)aminomethyl]phenyl}benzamide;
N-hydroxy-4-{3-[N-(benzyl)-N-(2-methoxyethyl)aminomethyl]phenyl}benzamide;
N-hydroxy-4-[3-(benzylaminomethyl)phenyl]benzamide;
N-hydroxy-4-[3-(2-indol-3-ylethylaminomethyl)phenyl]benzamide;
N-hydroxy-4-[3-(3,4-methylenedioxybenzylaminomethyl)phenyl}benzamide;
N-hydroxy-4-[3-(pyridin-4-ylmethylaminomethyl)phenyl}benzamide;
N-hydroxy-4-[3-(pyridin-3-yloxymethyl)phenyl}benzamide;
N-hydroxy-4-[3-(2-pyridin-3-ylethylaminomethyl)phenyl}benzamide;
N-hydroxy-4-[3-(phenyloxymethyl)phenyl}benzamide;
N-hydroxy-4-[3-(4-methylphenyloxymethyl)phenyl}benzamide;
N-hydroxy-4-[3-(4-chlorophenyloxymethyl)phenyl}benzamide;
N-hydroxy-4-[3-(3-phenylpropylaminomethyl)phenyl}benzamide;
N-hydroxy-4-[3-(phenylaminomethyl)phenyl}benzamide;
N-hydroxy-4-[3-(4-methylphenylaminomethyl)phenyl}benzamide;
N-hydroxy-4-[3-(4-chlorophenylaminomethyl)phenyl}benzamide;
N-hydroxy-[5-(morpholin-4-ylcarbonyl)-3-(4-methylphenylcarbonylamino)phenyl]-benzamide; or
N-hydroxy-4-[3-(pyridin-3-ylmethylcarbonylaminomethyl)phenyl]benzamide.

Presently preferred compounds are:
N-hydroxy-4-[3-(phenylsulfonylamino)phenyl]benzamide;
N-hydroxy-4-[3-(4-methoxyphenylsulfonylamino)phenyl]benzamide;
N-hydroxy-4-[3-(4-chlorophenylsulfonylamino)phenyl]benzamide;
N-hydroxy-4-[3-(phenylcarbonylamino)phenyl]benzamide;
N-hydroxy-4-[3-(2,4-dichlorophenylcarbonylamino)phenyl]benzamide;
N-hydroxy-4-[3-(4-methylphenylcarbonylamino)phenyl]benzamide;
N-hydroxy-4-[3-(3,4-methylenedioxyphenylcarbonylamino)phenyl]benzamide;
N-hydroxy-4-[3-(4-methoxyphenylcarbonylamino)phenyl]benzamide;
N-hydroxy-4-[3-(4-chlorophenylcarbonylamino)phenyl]benzamide;
N-hydroxy-4-[3-(4-methoxy-2-methylphenylcarbonylamino)phenyl]benzamide;
N-hydroxy-4-[3-(2,4-dimethylphenylcarbonylamino)phenyl]benzamide;
N-hydroxy-4-[3-(3,4-dichlorophenylcarbonylamino)phenyl]benzamide;
N-hydroxy-4-[3-(3,4-dimethoxyphenylcarbonylamino)phenyl]benzamide;
N-hydroxy-4-[3-(4-ethoxyphenylcarbonylamino)phenyl]benzamide;
N-hydroxy-4-[3-(4-fluorophenylcarbonylamino)phenyl]benzamide;
N-hydroxy-4-[3-(2,4-difluorophenylcarbonylamino)phenyl]benzamide;
N-hydroxy-4-[3-(benzylsulfonylamino)phenyl]benzamide;
N-hydroxy-4-[3-(4-dimethylphenylcarbonylamino)phenyl]benzamide;
N-hydroxy-[5-(N,N-dimethylaminocarbonyl)-3-(4-methoxyphenylcarbonylamino)-phenyl]benzamide;
N-hydroxy-[5-(phenylaminocarbonyl)-3-(4-methoxyphenylcarbonyl-amino)phenyl]-benzamide;
N-hydroxy-[5-(piperidin-1-ylcarbonyl)-3-(4-methylphenylcarbonylamino)-phenyl]benzamide;
N-hydroxy-[5-(morpholin-4-ylcarbonyl)-3-(2,4-dichlorophenylcarbonylamino)phenyl]-benzamide;
N-hydroxy-[5-(2-hydroxymethylpiperidin-1-ylcarbonyl)-3-(4-methylphenylcarbonyl-amino)phenyl]benzamide;
N-hydroxy-[5-(N,N-dimethylaminocarbonyl)-3-(4-methylphenylcarbonylamino)phenyl]-benzamide;
N-hydroxy-[5-(N-methoxy-N-methylaminocarbonyl)-3-(4-methylphenylcarbonyl-amino)phenyl]benzamide;
N-hydroxy-[5-(pyrrolidin-1-ylcarbonyl)-3-(2,4-dichlorophenylcarbonylamino)phenyl]-benzamide;
N-hydroxy-[5-(piperidin-1-ylcarbonyl)-3-(2,4-dichlorophenylcarbonylamino)phenyl]-benzamide;
N-hydroxy-[5-(N,N-dimethylaminocarbonyl)-3-(4-methylphenylcarbonylamino)-phenyl]-benzamide;
N-hydroxy-[5-(N,N-dimethylaminocarbonyl)-3-(3,4-dimethoxyphenylcarbonyl-amino)phenyl]benzamide;
N-hydroxy-5-(N,N-dimethylaminocarbonyl)-3-(2,4-dichlorophenylcarbonylamino)-phenyl]benzamide;
N-hydroxy-[5-(N,N-dimethylaminocarbonyl)-3-(4-chlorophenylcarbonylamino)phenyl]-benzamide;
N-hydroxy-[5-(N-ethyl-N-methylaminocarbonyl)-3-(4-methylphenylcarbonylamino)-phenylbenzamide;
N-hydroxy-[5-(N,N-diethylaminocarbonyl)-3-(4-methylphenylcarbonylamino)phenyl]-benzamide;
N-hydroxy-[5-(piperidin-1-ylcarbonyl)-3-(4-methylphenylcarbonylamino)phenyl]-benzamide;
N-hydroxy-[5-(morpholin-4-ylcarbonyl)-3-(4-methylphenylcarbonylamino)phenyl]-benzamide;
N-hydroxy-[5-(morpholin-4-ylcarbonyl)-3-(2,4-dichlorophenylcarbonylamino)phenyl]-benzamide;
N-hydroxy-[5-(N-methoxy-N-methylaminocarbonyl)-3-(4-methylphenylcarbonylamino)-phenyl]benzamide;
N-hydroxy-[5-(4-chlorophenylaminocarbonyl)-3-(4-methylphenylcarbonylamino)-phenyl]benzamide;
N-hydroxy-4-[3-(2-phenylethylaminocarbonylmethyl)phenyl]benzamide;
N-hydroxy-4-[3-(pyridin-3-ylmethylcarbonylaminomethyl)phenyl]benzamide;
N-hydroxy-4-[3-(2-methylphenylcarbonylamino)phenyl]benzamide;
N-hydroxy-4-[3-(2,4-dimethylphenylcarbonylamino)phenyl]benzamide;
N-hydroxy-4-[3-(2,5-dimethylphenylcarbonylamino)phenyl]benzamide; and
N-hydroxy-4-[3-(4-methoxyphenylcarbonylamino)phenyl]benzamide; or
   a pharmaceutically acceptable salt thereof.

### General Synthesis

Compounds of this invention can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compounds of formula I can be prepared by the procedure illustrated and described in Schemes A and B below.

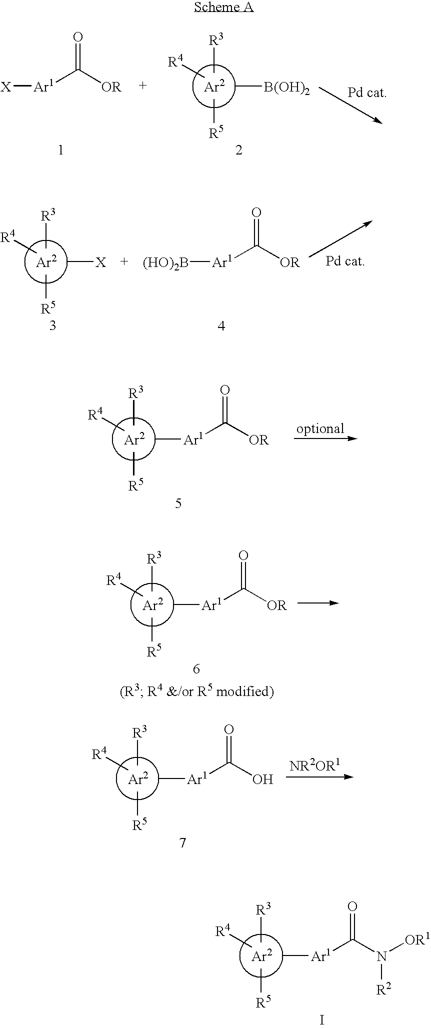

In general, compounds of Formula I where R¹-R⁵, Ar¹ and Ar² are as defined in the Summary of the Invention can be prepared by the procedure illustrated above and described in detail below.

Reaction of a compound of formula 1 (where X is halo such as chloro, bromo, or iodo and R is hydrogen or alkyl such as methyl, ethyl, and the like) with a boronic acid compound of formula 2 where Ar², R³-R⁵ are as defined in the Summary of the Invention provides a compound of formula 5. The coupling reaction is carried out in the presence of a palladium catalyst such as tetrakis(triphenylphosphine)palladium(0) and an inorganic base such as potassium carbonate. Suitable solvents are aromatic organic solvents such as benzene, toluene, and the like. Alternatively, compound 5 can be prepared by reacting a compound of formula 3 with a compound of formula 4 (where X, Ar², R³-R⁵ are as defined above) under the reaction conditions described above.

Compounds of formula 2 and 4 such as 2-, 3-methoxyphenylboronic acid, 2,4-dichlorophenylboronic acid, 3,5-dichlorophenylboronic acid, 3-formylphenylboronic acid, 5-chloro-2-methoxyphenylboronic acid, 3-nitrophenyl boronic acid, 4-methylbenzoate phenylboronic acid, 3-methoxyphenylboronic acid, 4-methoxycarbonylphenyl boronic acid, 4carboxyphenylboronic acid are commercially available. Heteroaryl boronic acids are also commercially available. Compounds of formula 1 and 3 are either commercially available or they can be prepared by methods well known in the art. For example, ethyl 4-bromobenzoate, 3-bromophenyl, methyl 4-bromophenylacetate, 3-bromobenzylcarbamic acid benzyl ester, and 4-bromobenzoic acid are commercially available. Ethyl 3-bromo-5-nitrobenzoate can be prepared from 3-nitrobenzoic acid by first brominating the acid with a suitable brominating agent such as N-bromosuccinimide in a mixture of trifluoroacetic acid and sulfuric acid to obtain 3-bromo-5-nitrobenzoic acid and then esterifying the carboxy group under conditions well known in the art. 3-Nitro-5-propoxymethylbromobenzene can be prepared from 3-nitrobenzaldehyde by first brominating it with N-bromosuccinimide as described above, to give 3-bromo-5-nitrobenzaldehyde. Reduction of the aldehyde group with a suitable reducing agent such as sodium borohydride then provides 3-bromo-5-nitrobenzylalcohol with upon treatment with propyl iodide in the presence of a suitable base such as sodium hydride then provides the desicred compound.

A compound of formula 5 can optionally be converted to a compound of formula 6 where any of the R³, R⁴, and R⁵ groups have been modified prior to converting it to a compound of Formula I. Some such examples are:

A compound of formula 5 where any of the R³, R⁴, and R⁵ is —X—R⁶ where X is —NR⁸—, —NR⁹CO—, NR¹¹SO₂—, —NR¹³CONR¹⁴— or —NR¹⁵SO₂NR¹⁶— where R⁶, R⁸, R⁹, R¹¹, R¹³, R¹⁴, R¹⁵, and R¹⁶ are as defined in the Summary of the invention can be prepared from a corresponding compound of formula 6 where any of the R³, R⁴, and R⁵ is nitro by first reducing the nitro group to an amino by methods well known in the art and then reacting the amino group with an alkylating agent, acylating agent, sulfonylating agent, carbamoyl halide or sulfamoyl halide respectively, under the reaction conditions well known in the art. For example, the above reactions can be carried out in the presence of a base such as triethylamine, N,N-diisopropyethylamine, pyridine, and the like and in a suitable solvent such as dichloromethane, tetrahydrofuran, dioxane, N,N-dimethylformamide, and the like. A detailed description of synthesis of compounds of Formula I by this procedure is provided in working examples below and in U.S. Pat. No. 6,136,844, the disclosure of which is incorporated herein by reference in its entirety.

A compound of formula 6 where any of the R³, R⁴, and R⁵ is -(alkylene)-NR⁷R⁸ where R⁸ is as defined in the Summary of the Invention and R⁷ is alkyl, optionally substituted phenylalkyl, optionally substituted phenoxyalkyl, optionally substituted phenylalkenyl, optionally substituted phenylaminoalkyl, optionally substituted heteroaralkyl, optionally substituted heteroaryloxyalkyl, optionally substituted heterocycloalkylalkyl, or cycloalkylalkyl can be prepared by reacting a compound of formula 5 where any of the R³, R⁴, and R⁵ is a formyl group with an amine of formula R⁷NH₂ under reductive amination reaction conditions.

A compound of formula 6 where any of the R³, R⁴, and R⁵ is hydroxy can be prepared from a corresponding compound of formula 5 where any of the R³, R⁴, and R⁵ is alkoxy by hydrolysis of the alkoxy group. Suitable dealkylating agents are boron trichloride, and the like.

A compound of formula 6 where any of the R³, R⁴, and R⁵ is —CONR¹⁰R⁶ or C₁₋₆-alkylene)-CONR¹⁴R⁷ where R⁶ and R⁷ are as defined in the Summary of the invention can be prepared by reacting a corresponding compound of formula 5 where any of the R³, R⁴, and R⁵ is a carboxy or carboxyalkyl group with a an amine under conditions described above. Compounds of formula 5 any of the R³, R⁴, and R⁵ is a carboxy or carboxyalkyl group can be prepared by reacting a compound of formula 3 such as bromobenzoic acid or bromophenylacetic acid with a compound of formula 4 where R is alkyl.

Compound 5 or 6 is converted to an acid of formula 7 under basic hydrolysis reaction conditions. Typical bases that are used are aqueous sodium hydroxide, potassium hydroxide, and the like. The reaction is carried out in an alcoholic solution such as methanol, ethanol, and the like. Compound 7 is then converted to a compound of Formula I by first reacting 5 with a halogenating agent such as oxalyl chloride, sulfonyl chloride, and then treating the resulting acid halide with a hydroxyamine of formula NHR²OR¹ where R¹ and R² are as defined in the Summary of the Invention. A compound of Formula I where R² is not hydrogen can also be prepared by reacting a corresponding compound of Formula I where R² is hydrogen with an alkylating agent under conditions well known in the art. Other methods of preparing compounds of Formula I from compound 7 are analogous to the methods disclosed in U.S. Pat. No. 5,998,412 the disclosure of which is incorporated herein by reference in its entirety.

A compound of Formula I can be converted to another compounds of Formula I. For example, a compound of Formula I where any of the R³, R⁴, and R⁵ is —X—R⁶ where X is —NR⁸—, —NR⁹CO—, NR¹¹SO₂—, —NR¹³CONR¹⁴— or —NR¹⁵SO₂NR¹⁶— where R⁶, R⁸, R⁹, R¹¹, R¹³, R¹⁴, R¹⁵, and R¹⁶ are as defined in the Summary of the invention can be prepared from a corresponding compound of Formula I where any of the R³, R⁴, and R⁵ is nitro as described previously. A compound of Formula I where any of the R³, R⁴, and R⁵ is —CONR¹⁰R⁶ or —(C₁₋₆-alkylene)-CONR¹⁴R⁷ where R⁶ and R⁷ are as defined in the Summary of the invention can be prepared by reacting a corresponding compound of Formula I where any of the R³, R⁴, and R⁵ is a carboxy or carboxyalkyl group as described previoualy

Alternatively, compounds of Formula I where Ar² is heteroaryl such as benzimidazole can be prepared as described in working Examples 5, 6 and 12 below.

Compound of Formula I can also be synthesized using hydroxylamine Wang resin as illustrated and described in Scheme B below.

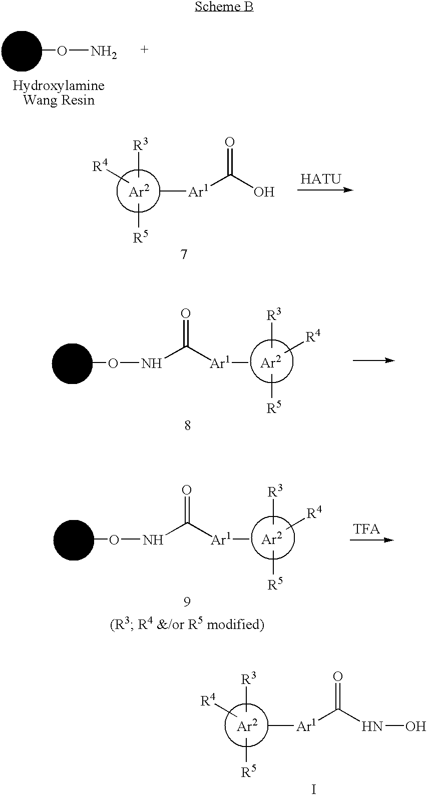

Treatment of pre-swelled hydroxylamine Wang resin with an acid of formula 7 where R³, R⁴ and R⁵ are as defined in the Summary of the Invention except a carboxy group in the presence of a coupling agent such as HATU and an organic base such as triethylamine, diisopropylethylamine, and the like provides a resin bound compound of formula 8. The reaction is carried out in suitable organic solvent such as N,N-dimethylformamide and at room temperature.

Compounds of formula 7 can be prepared as described in Scheme A above. Compound 8 is optionally converted to a compound of formula 9 where any of the R³, R⁴ and R⁵ groups are modified as described in Scheme A above.

Compound 8 or 9 is then treated with a strong acid such as trifluoroacetic acid to provide a compound of Formula I.

A detailed synthesis of a compound of Formula I utilizing this procedure is provided in Example 13 below.

### Utility

The compounds of this invention are inhibitors of histone deacetylase enzymes and are therefore useful in the treatment of proliferative diseases such as cancer and bipolar disorders.

### Testing

The ability of the compounds of this invention to inhibit histone deacetylase enzymes can be tested in vitro and in vivo assays described in biological assays Example 1 and 2 below.

### Administration and Pharmaceutical Compositions

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors.

Therapeutically effective amounts of compounds of Formula I may range from approximately 0.1-50 mg per kilogram body weight of the recipient per day; preferably about 0.5-20 mg/kg/day. Thus, for administration to a 70 kg person, the dosage range would most preferably be about 35 mg to 1.4 g per day.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral or parenteral using a convenient daily dosage regimen, which can be adjusted according to the degree of affliction. Oral compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of Formula I in combination with at least one pharmaceutically acceptable excipient. Acceptable excipients are non-toxic, aid administration, and do not adversely affect the therapeutic benefit of the compound of Formula I. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of Formula I based on the total formulation, with the balance being one or more suitable pharmaceutical excipients. Preferably, the compound is present at a level of about 1-80 wt %. Representative pharmaceutical formulations containing a compound of Formula I are described below.

As stated previously, the compounds of this invention can be administered in combination with known anti-cancer agents. Such known anti-cancer agents include the following: estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors. The compound of the present invention compounds are particularly useful when adminsitered in combination with radiation therapy. Preferred angiogenesis inhibitors are selected from the group consisting of a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, and an antibody to VEGF.

Preferred estrogen receptor modulators are tamoxifen and raloxifene.

“Estrogen receptor modulators” refers to compounds that interfere or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic agents” refer to compounds which cause cell death primarily by interfering directly with the cell's functioning or inhibit or interfere with cell myosis, including alkylating agents, tumor necrosis factors, intercalators, microtubulin inhibitors, and topoisomerase inhibitors.

Examples of cytotoxic agents include, but are not limited to, tirapazimine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichlorO(2-methyl-pyridine) platinum, benzylguanine, glufosfamide, GPX100, (trans, trans, trans)-bis-mu-(hexane-1,6-diamine)-mu-[diamine-platinum(II)]bis[diamine(chloro)platinum(II)]-tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycanninomycin, annamycin, galarubicin, elinafide, MEN10755, and 4-demethoxy-3-deamino-3-aziridinyl-4-methylsulphonyl-daunorubicin (see WO 00/50032).

Examples of microtubulin inhibitors include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR¹⁰⁹⁸⁸¹, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxymethyl-1H,12H-benzo[de]pyrano[3′,4′:b,7]-indolizino[1,2b]quinoline-10,13(9H,15H)dione, lurtotecan, 7-[2-(N-isopropylamino)-ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofurO(3′,4′: 6,7)colchic(2,3-d)-1,3-dioxol-6-one, 2,3-(methylenedioxy)-5-methyl-7-hydroxy-8-methoxybenzo[c]-phenanthridinium, 6,9-bis[(2-aminoethyl)-amino]benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[²(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]-adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)₄-formyl-6-methoxy-14-oxa-1,11-diazatetra cyclO(7.4.1.0.0)-tetradeca-2,4,6-trien-9-yl acetic acid ester, swainsonine, lometrexol, dexrazoxane, methioninase, 2′-cyano-2′-deoxy-N-4-palmitoyl-1-B-D-arabino furanosyl cytosine, and 3-aminopyridine-2-carboxaldehyde thiosemicarbazone. “Antiproliferative agents” also includes monoclonal antibodies to growth factors, other than those listed under “angiogenesis inhibitors”, such as trastuzumab, and tumor suppressor genes, such as p53, which can be delivered via recombinant virus-mediated gene transfer (see U.S. Pat. No. 6,069,134, for example).

“HMG-CoA reductase inhibitors” refers to inhibitors of 3-hydroxy-3-methylglutaryl-CoA reductase. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein. It has been reported that (Int. J. Cancer, 20;97(6):746-50, 2002) combination therapy with lovastatin, a HMG-CoA reductase inhibitor, and butyrate, an inducer of apoptosis in the Lewis lung carcinoma model in mice showed potentiating antitumor effects

Examples of HMG-CoA reductase inhibitors that may be used include but are not limited to lovastatin (MEVACOR®; see U.S. Pat. Nos. 4,231,938; 4,294,926; 4,319,039), simvastatin (ZOCOR®; see U.S. Pat. Nos. 4,444,784; 4,820,850; 4,916,239), pravastatin (PRAVACHOL®; see U.S. Pat. Nos. 4,346,227; 4,537,859; 4,410,629; 5,030,447 and 5,180,589), fluvastatin (LESCOL®; see U.S. Pat. Nos. 5,354,772; 4,911,165; 4,929,437; 5,189,164; 5,118,853; 5,290,946; 5,356,896), atorvastatin (LIPITOR®; see U.S. Pat. Nos. 5,273,995; 4,681,893; 5,489,691; 5,342,952) and cerivastatin (also known as rivastatin and BAYCHOL®; see U.S. Pat. No. 5,177,080). The structural formulas of these and additional IHMG-CoA reductase inhibitors that may be used in the instant methods are described at page 87 of M. Yalpani, “Cholesterol Lowering Drugs”, Chemistry & Industry, pp. 85-89 (Feb. 5, 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and □olchicin the use of such salts, esters, open-acid and lactone forms is included within the scope of this invention.

In HMG-CoA reductase inhibitors where an open-acid form can exist, salt and ester forms may preferably be formed from the open-acid, and all such forms are included within the meaning of the term “HMG-CoA reductase inhibitor” as used herein. Preferably, the HMG-CoA reductase inhibitor is selected from lovastatin and simvastatin, and most preferably simvastatin.

Herein, the term “pharmaceutically acceptable salts” with respect to the HMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenzimidazole, diethylamine, piperazine, and tris(hydroxymethyl) aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

“Prenyl-protein transferase inhibitor” refers to a compound which inhibits any one or any combination of the prenyl-protein transferase enzymes, including farnesyl-protein transferase (FPNase), geranylgeranyl-protein transferase type I (GGPTase-I), and geranylgeranyl-protein transferase type-II (GGPTase-II, also called Rab GGPTase). Examples of prenyl-protein transferase inhibiting compounds include (±)-6-[aminO(4chlorophenyl)(1-methyl-1H-imidazol-5-yl)methyl]4(3-chlorophenyl)-1-methyl-2(1H)-quinolinone, (−)-6-[aminO(4-chlorophenyl)(1-methyl-1H-imidazol-5-yl)methyl]4-(3-chloro phenyl)-1-methyl-2(1H)-quinolinone, (+)-6-[aminO(4-chlorophenyl)(1-methyl-1H-imidazol-5-yl)methyl]-4-(3-chloro phenyl)-1-methyl-2(1H)-quinolinone, 5(S)-n-butyl-1-(2,3-dimethylphenyl)-4-[1-(4-cyanobenzyl)-5-imidazolylmethy 1]-2-piperazinone, (S)-1-(3-chlorophenyl)-4-[1-(4-cyanobenzyl)-5-imidazolylmethyl]-5-[2-(ethanesulfonyl)-methyl)-2-piperazinone, 5(S)-n-butyl-1-(2-methylphenyl)-4-[1-(4-cyanobenzyl)-5-imidazolylmethyl]-2-piperazinone, 1-(3-chlorophenyl)-4-[1-(4-cyanobenzyl)-2-methyl-5-imidazolylmethyl]-2-piperazinone, 1-(2,2-diphenylethyl)-3-[N-(1-(4-cyanobenzyl)-1H-imidazol-5-ylethyl)carbamoyl]piperidine, 4-{5-[4-hydroxymethyl-4-(4-chloropyridin-2-ylmethyl)-piperidine-1-ylmethyl}-2-methylimidazol-1-ylmethyl}benzonitrile, 4-15-[4-hydroxymethyl-4(3-chlorobenzyl)-piperidine-1-ylmethyl]-2-methylimidazol-1-ylmethyl}benzonitrile, 4-{3-[4-(2-oxo-2H-pyridin-1-yl)benzyl]-3H-imidazol-4-ylmethyl}benzonitrile, 4-{3-[4-(5-chloro-2-oxo-2H-[1,2′]bipyridin-5′-ylmethyl]-3H-imidazol-4-ylmethyl}benzonitrile, 4-{3-[4-(2-oxo-2H-[1,2′]bipyridin-5′-ylmethyl]-3H-imidazol-4-ylmethyl}benzonitrile, 4-[3-(2-oxo-1-phenyl-1,2-dihydropyridin-4-ylmethyl)-3H-imidazol-4-ylmethyl}benzonitrile, 18,19-dihydro-19-oxo-5H,17H-6,10:12,16-dimetheno-1H-imidazo[4,3-c][1,11,4]dioxa-azacyclononadecine-9-carbonitrile, (±)-19,20-dihydro-19-oxo-5H-18,21-ethano-12,14-etheno-6,10-metheno-22H-benzo[d]imidazo[4,3-k][1,6,9,12]-oxatriaza-cyclooctadecine-9-carbonitrile, 19,20-dihydro-19-oxo-5H,17H-18,21-ethano-6,10:12,16-dimetheno-22H-imidazo[3,4-h][1,8,11,14]oxatriazacyclo-eicosine-9-carbonitrile, and (±)-19,20-dihydro-3-methyl-19-oxo-5H-18,21-ethano-12,14-etheno-6,10-met heno-22H-benzo[d]imidazo[4,3-k][1,6,9,12]oxa-triazacyclooctadecine-9-carbonitrile.

Other examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. Nos. 5,420,245, 5,523,430, 5,532,359, 5,510,510, 5,589,485, 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see J. of Cancer, Vol. 35, No. 9, pp. 1394-1401 (1999).

Examples of HIV protease inhibitors include amprenavir, abacavir, CGP-73547, CGP-61755, DMP-450, indinavir, nelfinavir, tipranavir, ritonavir, saquinavir, ABT-378, AG 1776, and BMS-232, 632. Examples of reverse transcriptase inhibitors include delaviridine, efavirenz, GS-840, HB Y097, lamivudine, nevirapine, AZT, 3TC, ddC, and ddI. It has been reported (Nat. Med.;8(3):225-32, 2002) that HIV protease inhibitors, such as indinavir or saquinavir, have potent anti-angiogenic activities and promote regression of Kaposi sarcoma

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR20), inhibitors of epidermal-derived, fibroblast-derived, or platelet derived growth factors, MMP (matrix metalloprotease) inhibitors, integrin blockers, interferon-, interleukin-12, pentosan polysulfate, cyclooxygenase inhibitors, including nonsteroidal anti-inflammatories (NSAIDs) like aspirin and ibuprofen as well as selective cyclooxygenase-2 inhibitors like celecoxib, valecoxib, and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin., Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Ind. J. Mol. Med., Vol. 2, p. 715 (1998); J. Biol. Chem., Vol. 274, p. 9116 (1999)), carboxyamidotriazole, combretastatin A4, squalamine, 6-O-chloroacetyl-carbonyl)-fumagillol, thalidomide, angiostafin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

As described above, the combinations with NSAD)'s are directed to the use of NSAID's which are potent COX-2 inhibiting agents. For purposes of this specification an NSAID is potent if it possess an IC₅₀ for the inhibition of COX-2 of 1 μM or less as measured by the cell or microsomal assay known in the art.

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by the cell or microsomal assay disclosed hereinunder. Such compounds include, but are not limited to those disclosed in U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, U.S. Pat. No. 5,710,140, issued Jan. 20, 1998, WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999, all of which are hereby incorporated by reference. Other examples of specific inhibitors of COX-2 include those disclosed in U.S. Pat. No. 6,313,138 the disclosure of which is incorporated herein by reference in its entirety.

General and specific synthetic procedures for the preparation of the COX-2 inhibitor compounds described above are found in U.S. Pat. No. 5,474,995, issued Dec. 12, 1995, U.S. Pat. No. 5,861,419, issued Jan. 19, 1999, and U.S. Pat. No. 6,001,843, issued Dec. 14, 1999, all of which are herein incorporated by reference.

Compounds that have been described as specific inhibitors of COX-2 and are therefore useful in the present invention include, but are not limited to, the following: 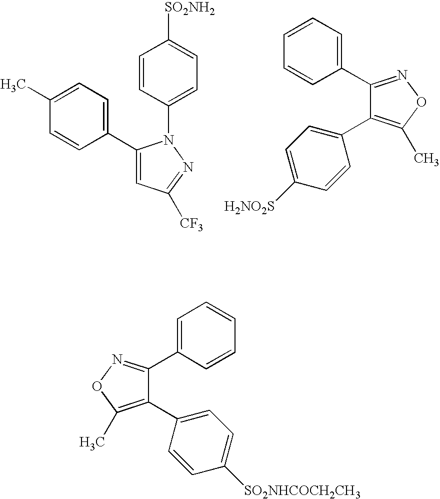
or a pharmaceutically acceptable salt thereof.

Compounds which are described as specific inhibitors of COX-2 and are therefore useful in the present invention and methods of synthesis thereof, can be found in the following patents, pending applications and publications, which are herein incorporated by reference: WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999.

Compounds which are specific inhibitors of COX-2 and are therefore useful in the present invention, and methods of synthesis thereof, can be found in the following patents, pending applications and publications, which are herein incorporated by reference: U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, and U.S. Pat. No. 5,710,140, issued Jan. 20, 1998.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]-methyl]-1H-1,2,3-triazole-4-carboxamide, CM101, squalamine, combretastatin, RPI4610, NX31838, sulfated mannopentose phosphate, 7,7-(carbonyl-bis[imino-N-methyl-4,2-pyrrolocarbonyl-imino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counter-act binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆; αᵥβ₈, α₁β₁, α₂β₁, α₅β₁, α₆β₁, and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁, and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)₄-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, ST1571, CEP2563, 4-(3-chlorophenylamino)-5,6-dimethyl-7H-pyrrolo [2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, SU11248, ST1571A, N-4-chlorophenyl-4-(4-pyridylmethyl)-1-phthalazinamine, and EMD 121974.

The instant compounds are also useful, alone or in combination with platelet fibrinogen receptor (GP IIb/IIIa) antagonists, such as tirofiban, to inhibit metastasis of cancerous cells. Tumor cells can activate platelets largely via thrombin generation. This activation is associated with the release of VEGF. The release of VEGF enhances metastasis by increasing extravasation at points of adhesion to vascular endothelium (Amirkhosravi, Platelets 10, 285-292, 1999). Therefore, the present compounds can serve to inhibit metastasis, alone or in combination with GP IIb/IIIa) antagonists. Examples of other fibrinogen receptor antagonists include abciximab, eptifibatide, sibrafiban, lamifiban, lotrafiban, cromofiban, and CT50352.

If formulated as a fixed dose, such combination products employ the compounds of this invention within the dosage range described above and the other pharmaceutically active agent(s) within its approved dosage range. Compounds of the instant invention may alternatively be used sequentially with known pharmaceutically acceptable agent(s) when a combination formulation is inappropriate.

The term administration and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The compounds of the instant invention may also be co-administered with other well known therapeutic agents that are selected for their particular usefulness against the condition that is being treated. For example, the compounds of the instant invention may also be co-administered with other well known cancer therapeutic agents that are selected for their particular usefulness against the condition that is being treated. Included in such combinations of therapeutic agents are combinations of the farnesyl-protein transferase inhibitors disclosed in U.S. Pat. No. 6,313,138 and an antineoplastic agent. It is also understood that such a combination of antineoplastic agent and inhibitor of farnesyl-protein transferase may be used in conjunction with other methods of treating cancer and/or tumors, including radiation therapy and surgery.

Examples of an antineoplastic agent include, in general, microtubule-stabilizing agents (such as paclitaxel (also known as Taxol®), docetaxel (also known as Taxotere® epothilone A, epothilone B, desoxyepothilone A, desoxyepothilone B or their derivatives); microtubule-disruptor agents; alkylating agents, anti-metabolites; epidophyllotoxin; an antineoplastic enzyme; a topoisomerase inhibitor; procarbazine; mitoxantrone; platinum coordination complexes; biological response modifiers and growth inhibitors; hormonal/anti-hormonal therapeutic agents and haematopoietic growth factors.

Example classes of antineoplastic agents include, for example, the anthracycline family of drugs, the vinca drugs, the mitomycins, the bleomycins, the cytotoxic nucleosides, the taxanes, the epothilones, discodermolide, the pteridine family of drugs, diynenes and the podophyllotoxins. Particularly useful members of those classes include, for example, doxorubicin, carminomycin, daunorubicin, aminopterin, methotrexate, methopterin, dichloro-methotrexate, mitomycin C, porfiromycin, Herceptin®, Rituxan®, 5-fluorouracil, 6-mercaptopurine, gemcitabine, cytosine arabinoside, podophyllotoxin or podo-phyllotoxin derivatives such as colchicines, etoposide, etoposide phosphate or teniposide, melphalan, vinblastine, vincristine, leurosidine, vindesine, leurosine, paclitaxel and the like. Other useful antineoplastic agents include estramustine, cisplatin, carboplatin, cyclophosphamide, bleomycin, tamoxifen, ifosamide, melphalan, hexamethyl melamine, thiotepa, cytarabin, idatrexate, trimetrexate, dacarbazine, Lasparaginase, camptothecin, CPT-11, topotecan, ara-C, bicalutamide, flutamide, leuprolide, pyridobenzoindole derivatives, interferons and interleukins. The preferred class of antineoplastic agents is the taxanes and the preferred antineoplastic agent is paclitaxel.

Radiation therapy, including x-rays or gamma rays that are delivered from either an externally applied beam or by implantation of tiny radioactive sources, may also be used in combination with the compounds of this invention alone to treat cancer.

### EXAMPLES

The following preparations and examples are given to enable those skilled in the art to more clearly understand and to practice the present invention. They should not be considered as limiting the scope of the invention, but merely as being illustrative and representative thereof.

### SYNTHETIC EXAMPLES

### Example 1

### Synthesis of N-hydroxy-4-(3-methoxyphenyl)benzamide

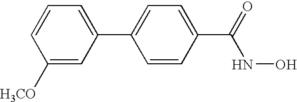 Step 1

To a prestirred solution of ethyl 4-bromo benzoate (1.2 g, 5.2 mmol) and tetrakis(triphenylphosphine)palladium(0) (0.3 g, 0.3 mmol) in toluene (15 ml) and ethanol (3 ml) (30 minutes) was added 3-methoxyphenylboronic acid (1.2 g, 7.8 mmol) and K₂CO₃ (3.6 g, 26 mmol). The resulting slurry was heated to 75° C. for 3 hr then cooled to room temperature and diluted with ether (200 ml) and washed with water (300 ml). The organic layer was dried (MgSO₄), filtered and concentrated to a brown oil. Flash chromatography (10% ethyl acetate/hexanes) provided 0.97 g (73%) of ethyl 4-(3-methoxyphenyl)benzoate as a white solid.

Step 2

To a solution of ethyl 4-(3-methoxyphenyl)benzoate (0.97 g, 3.8 mmol) in methanol (20 ml) and THF (10 ml) was added NaOH (0.8 g dissolved in 5 ml of H₂O). The solution was stirred 6 hr at room temperature, then acidified to pH=2 with 1N HCl, diluted with H₂O (150 ml) and extracted into ethyl acetate (150 ml). The organic layer was dried (MgSO₄), filtered and concentrated to collect 0.79 g (92%) of 4-(3-methoxyphenyl)benzoic acid as a white solid.

Step 3

To a solution of 4-(3-methoxyphenyl)benzoic acid (−0.3 g, 1.5 mmol) and DMF (3 drops) in THF (10 ml) was added oxalyl chloride (0.26 ml, 3 mmol). After stirring the solution for 45 minutes at room temperature, the solvent was removed in vacuo and then the resulting oil was dried under high vacuum. The oil was dissolved in THF (10 ml), cooled with an ice bath and then 50% aq. hydroxylamine (3 ml) was added. The cooling bath was removed and the light orange solution was stirred 30 minutes at room temperature. The reaction solution was then diluted with 1N HCl (100 ml) and extracted with ethyl acetate (100 ml). The solvent was dried (MgSO₄), filtered and concentrated to give the title compound in a 78% yield. ¹H NMR (DMSO-d₆): 11.25 (1H, s), 9.05 (1H, s), 7.82 (2H, d, J=8.4 Hz), 7.74 (2H, d, J=8.4 Hz), 7.39 (1H, t, J=7.7 Hz), 7.25 (2H, m), 6.96 (1H, m), 3.82 (3H, s); MS: 244.4 (M+1); 242.4 (M−1).

Proceeding as described in Example 1 above but using appropriate starting materials the following compounds of Formula I were prepared:

N-hydroxy-4-(2-methoxyphenyl)benzamide: ¹H NMR (DMSO-d₆): 11.21 (1H, s), 7.77 (2H, d, J=8.4 Hz), 7.52 (2H, d, J=8.4 Hz), 7.33 (2H, m), 7.12 (1H, d, J=8.2 Hz), 7.04 (1H, t, J=8.2 Hz), 3.76 (3H, s); MS: 244.4 (M+1); 242.4 (M−1).

N-hydroxy-4-(2,4-dichlorophenyl)benzamide: ¹H NMR (DMSO-d₆): 11.30 (1H, s), 9.10 (1H, s), 7.83 (2H, d, J=8.2 Hz), 7.76 (1H, s), 7.52-7.44 (4H, m); MS: 282.2 (M+1); 280.2 (M−1).

N-hydroxy-4-(3,5-dichlorophenyl)benzamide: ¹H NMR (DMSO-d₆): 11.32 (1H, br s), 7.85 (4H, s), 7.80 (2H, s), 7.64 (1H, m); (4H, m); MS: 282.2 (M+1); 280.2 (M−1).

N-hydroxy-4-(5-chloro-2-methoxyphenyl)benzamide: ¹H NMR (DMSO-d₆): 11.25 (1H, s), 9.47 (1H, s), 7.77 (2H, d, J=8.2 Hz), 7.54 (2H, d, J=8.2 Hz) 7.40 (1H, dd, J=9.0 Hz, J=2.7 Hz), 7.34 (1H, d, J=2.7 Hz), 7.15 (1H, d, J=9.0 Hz), 3.76 (3H, s); MS: 278.2 (M+1); 276.4 (M−1).

N-hydroxy-4-(3-isopropyl)benzamide: ¹H NMR (DMSO-d₆): 11.26 (1H, s), 9.08 (1H, br s), 7.83 (2H, d, J=8.2 Hz), 7.64 (2H, d, J=8.2 Hz), 7.55 (1H, s), 7.49 (1H, d, J=7.7 Hz), 7.39 (1H, t, J=7.7 Hz), 7.27 (1H, d, J=7.7 Hz), 2.94 (1H, m), 1.25 (6H, d, J=6.9 Hz); MS: 256.4 (M+1); 254.4 (M−1).

N-hydroxy-4-(3-nitrophenyl)benzamide: ¹H NMR (DMSO-d₆): 11.33 (1H, s), 9.11 (1H, s), 8.48 (1H, s), 8.23 (2H, m), 7.89 (4H, s), 7.78 (1H, t, J=8.2 Hz); MS: 259.2 (M+1); 257.4 (M−1).

N-hydroxy-4-(2,3-dimethylphenyl)benzamide: ¹H NMR (DMSO-d₆): 11.23 (1H, s), 9.05 (1H, s), 7.80 (2H, d, J=8.2 Hz), 7.38 (2H, d, J=8.2 Hz), 7.17 (2H, m), 7.03 (1H, d, J=8.0 Hz), 2.30 (3H, s), 2.08 (3H, s); MS: 242.2 (M+1); 240.2 (M−1).

### Example 2

### Synthesis of N-hydroxy-4-(3-benzenesulfonylaminophenyl)benzamide

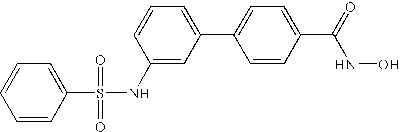 Step 1

A mixture of ethyl 4-(3-nitrophenyl)benzoate (0.6 g, 2.2 mmol) (prepared using the method of Example 1, Step 1) and palladium (10% on charcoal, 100 mg) was stirred under a hydrogen atmosphere for 4 hr and then the suspension was filtered through a plug of Celite® to remove the palladium. Concentration of the solvent provided of 0.5 g (93%) of ethyl 4-(3-amino-phenyl)benzoate.

Step 2

A solution of ethyl 4-(3-aminophenyl)benzoate (0.24 g, 1.0 mmol), TEA (0.7 ml) and benzenesulfonyl chloride (0.26 ml, 2.0 mmol) in THF (10 ml) was strirred at room temperature for 24 hr, followed by addition of NaOH (0.8 g dissolved in 5 ml H₂O) and methanol (5 ml). The solution was stirred an additional 12 hr, then diluted with H₂O (100 ml) and extracted with ether (2×100 ml). The aqueous layer was acidified (pH=2) and then extracted with ethyl acetate (100 ml). The organic layer was dried (MgSO₄), filtered and concentrated to collect 0.33 g (93%) of 4-(3-phenylsulfonylamino)benzoic acid which was converted to the title compound following the procedure described in Example 1, step 3, above in an 82% yield. ¹H NMR (DMSO-d₆): 11.20 (1H, s), 10.38 (1H, s), 7.78-7.73 (4H, m), 7.55-7.48 (5H, m), 7.29 (3H, m), 7.05 (1H, m): MS: 369.2 (M+1); 367.2 (M−1).

Proceeding a described in Example 2 above but using suitable starting materials provided the following compounds of Formula 1:

N-hydroxy-4-(3-methanesulfonylaminophenyl)benzamide: ¹H NMR (DMSO-d₆): 11.23 (1H, br s), 9.81 (1H, s), 7.79 (2H, d, J=8.4 Hz), 7.62 (2H, d, J=8.4 Hz), 7.39 (3H, m), 7.18 (1H, m), 2.97 (3H, s): MS: 307.2 (M+1); 305.4 (M−1).

N-hydroxy-4-(4-benzenesulfonylaminophenyl)benzamide. ¹H NMR (DMSO-d₆): 10.50 (1H, s), 7.82-7.76 (4H, m), 7.66-7.54 (7H, m), 7.29 (1H, d, J=8.7 Hz): MS: 369.4 (M+1); 367.2 (M−1).

N-hydroxy-4-[3-(4-methoxybenzenesulfonylamino)phenyl]benzamide: ¹H NMR (DMSO-d₆): 11.26 (1H, s), 10.29 (1H, s), 9.06 (1H, s), 7.82 (2H, d, J=8.4 Hz), 7.73 (2H, d, J=6.9 Hz), 7.58 (2H, d, J=8.4 Hz), 7.34 (3H, m), 7.06 (3H, m), 3.76 (3H, s); MS: 399.2 (M+1); 397.2 (M−1).

N-hydroxy-4-(3-benzylsulfonylaminophenyl)benzamide: ¹H NMR (DMSO-d₆): 11.28 (1H, s), 9.95 (1H, s), 7.85 (2H, d, J=8.4 Hz), 7.65 (2H, d, J=8.4 Hz),7.41-7.19 (9H, m), 4.52 (2H, s); MS: 383.6 (M+1); 381.4 (M−1).

### Example 3

### Synthesis of N-hydroxy-4-[3-(3,4-dichlorophenylcarbonylamino)phenyl]benzamide

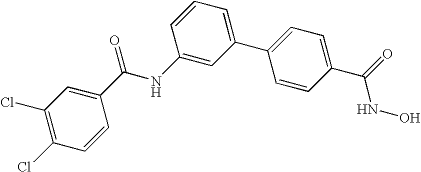 Step 1

To a solution of ethyl 4-(3-nitrophenyl)benzoate (0.5 g, 1.8 mmol) in methanol (20 ml) was added NaOH (0.8 g dissolved in 7 ml of H₂O). The solution was stirred 6 hr at room temperature, then acidified to pH=2 with 1N HCl, diluted with H₂O (150 ml). The resulting white solid was collected by filtration and dried under high vacuum to provide 0.39 g (87%) 4-(3-nitrophenyl)benzoic acid.

Step 2

To a solution of 4-(3-nitrophenyl)benzoic acid (2.7 g, 11.0 mmol), O-tert-butylhydroxylamine hydrochloride (1.5 g, 12 mmol) and TEA (7.7 ml) in DMF (50 ml) was added BOP-Cl (5.1 g, 11.6 mmol). After stirring 12 hr at room temperature, the solution was diluted with ethyl acetate (300 ml) and washed with 1N HCl (2×200 ml), then mild NaHCO₃ (2×200 ml). The organic layer was dried (MgSO₄), filtered and concentrated to collect 3.4 g (98%) of %) N-tert-butoxy-4-(3-nitrophenyl)benzamide.

Step 3

A mixture of N-tert-butoxy-4-(3-nitrophenyl)benzamide (0.5 g, 1.5 mmol) and palladium (10% on charcoal, 100 mg) was stirred under a hydrogen atmosphere for 35 min., and then the suspension was filtered through a plug of Celite to remove the palladium. Concentration of the solvent resulted in the isolation of 0.42 g (96%) of N-tert-butoxy 4(3-aminophenyl)benzamide.

Step 4A

To a solution of N-tert-butoxy-4-(3-aminophenyl)benzamide (0.10 g, 0.35 mmol) and TEA (0.25 ml) in THF (5 ml) was added 3,4-dichlorobenzoyl chloride (77 mg, 0.37 mmol). The reaction was stirred 1 hr at room temperature then diluted with ethyl acetate (50 ml) and washed with 1N HCl (100 ml), then mild NaHCO₃ (100 ml). The organic layer was dried (MgSO₄), filtered and concentrated to collect 0.17 g of an off-white solid. The solid was stirred in THF (3 ml) and then 30% HBr in acetic acid (2 ml) was added. After 1 hr at room temperature the solvent was removed and the residue was triturated with ether. The resulting off-white solid was collected by filtration and dried to provide 0.1 g (75%) of N-hydroxy-4-[3-(3,4-dichlorophenylcarbonyl-amino)phenyl]benzamide. ¹H NMR (DMSO-d₆): 10.44 (1H, s), 8.19 (1H, d, J=2.0 Hz), 8.07 (1H, s), 7.91 (1H, m), 7.79 (4H, m), 7.68 (2H, m), 7.43 (2H, m). MS: 401.4 (M+1); 398.8 (M−1).

Step 4B

To a solution of N-tert-butoxy-4-(3-aminophenyl)benzamide (0.10 g, 0.35 mmol), phenylacetic acid (53 mg, 0.39 mmol) and TEA (0.25 ml) in THF (5 ml) was added BOP-Cl (0.16 g, 0.37 mmol). The reaction mixture was stirred 1 hr at room temperature then diluted with ethyl acetate (50 ml) and washed with 1N HCl (100 ml), then mild NaHCO₃ (100 ml). The organic layer was dried (MgSO₄), filtered and concentrated to collect an off-white solid. The solid was stirred in THF (3 ml) and then 30% HBr in acetic acid (2 ml) was added. After 1 hr at room temperature the solvent was removed and the residue was triterated with ether. The resulting off-white solid was collected by filtration and dried to provide 75 mg (62%) of N-hydroxy-4-(3-benzylcarbonylaminophenyl)benzamide. ¹H NMR (DMSO-d₆):11.27 (1H, s), 10.30 (1H, s), 9.06 (1H, s), 7.98 (1H, s), 7.84 (2H, d, J=8.4 Hz), 7.67 (2H, d, J=8.4 Hz), 7.59 (1H, m), 7.41-7.25 (7H, m), 3.67 (2H, s). MS: 347.2 (M+1); 345.2 (M−1).

Proceeding a described in Example 3, Steps 1-3 and 4A above, but using suitable starting materials provided the following compounds of Formula I:

N-hydroxy-4-(3-phenylcarbonylaminophenyl)benzamide. ¹H NMR (DMSO-d₆): 11.22 (1H,s), 10.30 (1H,s), 8.10 (1H, s), 7.91 (1H, d, J=8.2 Hz), 7.79 (3H, m), 7.67 (2H, d, J=8.4 Hz), 7.55-7.40 (5H, m). MS: 333.6 (M+1); 331.4 (M−1).

N-hydroxy-4-[3-(3-methoxyphenylureido)phenyl]benzamide. ¹H NMR (DMSO-d₆): 11.28 (1H, s), 7.85 (2H, d, J=8.4 Hz), 7.69 (2H, d, J=8.4 Hz), 7.45-7.18 (6H, m), 6.94 (1H, m), 6.58 (1H, m), 3.73 (3H, s). MS: 378.6 (M+1); 376.2 (M−1).

N-hydroxy-4-[3-(3,4-dimethoxyphenylcarbonylamino)phenyl]benzamide. ¹H NMR (DMSO-d₆): 11.27 (1H, s), 10.17 (1H, s), 9.06 (1H, s), 8.12 (1H, s), 7.85 (2H, d, J=7.9 Hz), 7.80 (1H, m), 7.72 (2H, d, J=7.9 Hz), 7.63 (1H, m), 7.55 (1H, d, J=2.0 Hz), 7.44 (2H, m), 7.08 (1H, d, J=8.4 Hz), 3.84 (3H, s), 3.80 (3H, s). MS: 393.6 (M+1); 391.4 (M−1).

N-hydroxy-4-[3-(3-fluorophenylcarbonylamino)phenyl]benzamide. ¹H NMR (DMSO-d₆): 11.27 (1H, s), 10.42 (1H, s), 8.14 (1H, s), 7.90-7.70 (6H, m), 7.60 (2H, m), 7.45 (3H, m). MS: 351.2 (M+1); 349.0 (M−1).

N-hydroxy-4-[3-(2,4-dichlorophenylcarbonylamino)phenyl]benzamide. ¹H NMR (DMSO-d₆): 11.22 (1H, s), 10.61 (1H, s), 8.10 (1H, s), 7.80 (2H, d, J=8.4 Hz), 7.72 (1H, d, J=2.0 Hz), 7.64 (4H, m), 7.51 (1H, dd, J=8.4 Hz, J=2.0 Hz), 7.41 (2H, m). MS: 401.1 (M+1); 399.2 (M−1).

N-hydroxy-4-[3-(4-methylphenylcarbonylamino)phenyl]benzamide. ¹H NMR (DMSO-d₆): 10.27 (1H, s), 8.15 (1H, s), 7.85 (5H, m) 7.71 (2H, d, J=8.4 Hz), 7.50-7.31 (4H, m). 2.39 (3H, s). MS: 347.0 (M+1); 345.2 (M−1).

N-hydroxy-4-[3-(3-trifluoromethylphenylcarbonylamino)phenyl]benzamide. ¹H NMR (DMSO-d₆): 11.28 (1H, s), 10.57 (1H, s), 8.30 (2H, m), 8.11 (1H, s), 7.97 (1H, d, J=8.4 Hz), 7.90-7.70 (6H, m), 7.49 (2H, m). MS: 401.1 (M+1); 399.2 (M−1).

N-hydroxy-4-[3-(3,4-methylenedioxyphenylcarbonylamino)phenyl]benzamide. ¹H NMR (DMSO-d₆): 11.28 (1H, s), 10.17 (1H, s), 9.07 (1H, s), 8.13 (1H, s), 7.88 (2H, d, J=8.4 Hz), 7.82 (1H, m), 7.72 (2H, d, J=8.4 Hz), 7.60 (1H, d, J=7.9 Hz), 7.54 (1H, s), 7.45 (2H, m), 7.08 (1H, d, J=8.2 Hz), 6.14 (2H, s). MS: 377.2 (M+1); 375.0 (M−1).

N-hydroxy-4-[3-(3,5-dichlorophenylcarbonylamino)phenyl]benzamide. ¹H NMR (DMSO-d₆): 11.27 (1H, s), 10.51 (1H, s), 8.10 (1H, s), 8.01 (2H, d, J=1.7 Hz), 7.85 (4H, m), 7.71 (2H, d, J=8.4 Hz), 7.48 (2H, d, J=5.0 Hz). MS: 401.2 (M+1); 399.2 (M−1).

N-hydroxy-4-[3-(phenoxymethylcarbonylamino)phenyl]benzamide. ¹H NMR (DMSO-d₆): 11.27 (1H, s), 10.18 (1H, s), 8.01 (1H, s), 7.85 (2H, d, J=8.4 Hz), 7.70 (2H, d, J=8.4 Hz), 7.64 (1H, m), 7.44 (2H, m), 7.31 (2H, m), 7.00 (3H, m), 4.71 (2H, s). MS: 363.4 (M+1); 361.4 (M−1).

N-hydroxy-4-[3-(4-fluorophenylcarbonylamino)phenyl]benzamide. ¹H NMR (DMSO-d₆): 11.28 (1H, s), 10.37 (1H, s), 8.14 (1H, s), 8.07 (2H, m), 7.87 (2H, d, J=8.4 Hz), 7.82 (1H, m), 7.72 (2H, d, J=8.4 Hz), 7.48-7.36 (4H, m). MS: 351.4 (M+1); 349.0 (M−1).

Proceeding a described in Example 4, Steps 1-3 and 4B above, but using suitable starting materials provided the following compounds of Formula I:

N-hydroxy-443-(2-phenylethylcarbonylaminophenyl)benzamide: ¹H NMR (DMSO-d₆): 11.27 (1H, s), 10.03 (1H, s), 7.95 (1H, s), 7.84 (2H, d, J=8.4 Hz), 7.67 (2H, d, J=8.4 Hz), 7.57 (1H, m), 7.40-7.15 (7H, m), 2.92 (2H, t, J=7.4 Hz), 2.64 (2H, t, J=7.4 Hz). MS: 361.4 (M+1); 359.2 (M−1).

N-hydroxy-4-{3-[2-(4-trifluormethylphenyl)ethylcarbonylamino]phenyl}benzamide: ¹H NMR (DMSO-d₆): 10.07 (1H, s), 7.94 (1H, s), 7.84 (2H, d, J=8.4 Hz), 7.66 (4H, m), 7.59 (1H, m), 7.52 (2H, m), 7.37 (2H, m) 3.01 (2H, t, J=7.4 Hz), 2.69 (2H, t, J=7.4 Hz). MS: 429.2 (M+1); 427.2 (M−1).

N-hydroxy-4-{3-[2-(4-methoxyphenyl)ethylcarbonylamino]phenyl}benzamide: ¹H NMR (DMSO-d₆): 11.26 (1H, s), 10.01 (1H, s), 7.95 (1H, s), 7.83 (2H, d, J=8.4 Hz), 7.65 (2H, d, J=8.4 Hz), 7.57 (1H, m), 7.38 (2H, m), 7.15 (2H, d, J=8.6 Hz), 6.83 (2H, d, J=8.6 Hz) 3.69 (3H, s), 2.85 (2H, t. J=7.4 Hz), 2.60 (2H, t, J=7.4 Hz). MS: 391.4 (M+1); 389.2 (M−1).

N-hydroxy-4-[3-(4-methylbutylcarbonylamino)phenyl]benzamide: ¹H NMR (DMSO-d₆): 11.25 (1H, s), 9.98 (1H, s), 9.05 (1H, s), 7.95 (1H, s), 7.82 (2H, d, J=8.4 Hz), 7.62 (2H, d, J=8.4 Hz), 7.57 (1H, m), 7.35 (2H, m), 2.31 (2H, t, J=7.4 Hz), 1.49 (3H, m), 0.88 (6H, d, J=6.2 Hz). MS: 327.6 (M+1); 325.6 (M−1).

N-hydroxy-4-[3-(thiophen-2-ylmethylcarbonylamino)phenyl]benzamide: ¹H NMR (DMSO-d₆): 11.26 (1H, s), 10.32 (1H, s), 9.05 (1H, s), 7.96 (1H, s), 7.83 (2H, d, J=8.4 Hz), 7.67 (2H, d, J=8.4 Hz), 7.57 (1H, m), 7.39 (3H, m), 6.98 (2H, m), 3.89 (2H, s). MS: 353.4 (M+1); 351.4 (M−1).

N-hydroxy-4-{3-[2-(2,4-dichlorophenyl)ethylcarbonylamino]phenyl}benzamide: ¹H NMR (DMSO-d₆): 11.27 (1H, s), 10.08 (1H, s), 9.06 (1H, s), 7.94 (1H, s), 7.84 (2H, d, J=8.4 Hz), 7.67 (2H, d, J=8.4 Hz), 7.59 (2H, m), 7.39 (4H, m), 3.01 (2H, t, J=7.2 Hz), 2.66 (2H, t, J=7.2 Hz). MS: 429.2 (M+1); 427.2 (M−1).

N-hydroxy-4-{3-[2-(3,4-methylenedioxyphenyl)ethylcarbonylamino]phenyl)benzamide: ¹H NMR (DMSO-d₆): 11.26 (1H, s), 10.00 (1H, s), 7.93 (1H, s), 7.84 (2H, d, J=8.4 Hz), 7.65 (2H, d, J=8.4 Hz), 7.56 (1H, m), 7.36 (2H, m), 6.82 (1H, m), 6.78 (1H, s), 6.69 (1H, m), 5.94 (2H, s), 2.83 (2H, t, J=7.2 Hz), 2.59 (2H, t, J=7.2 Hz). MS: 405.6 (M+1); 403.4 (M−1).

N-hydroxy-4-[3-(4-trifluoromethylphenylcarbonylamino)phenyl]benzamide. ¹H NMR (DMSO-d₆): 11.28 (1H, s), 10.57 (1H, s), 9.06 (1H, s), 8.16 (3H, m), 7.95-7.71 (7H, m), 7.49 (2H, m). MS: 401.2 (M+1); 399.4 (M−1).

N-hydroxy-4-[3-(4-ethoxyphenylcarbonylamino)phenyl]benzamide. ¹H NMR (DMSO-d₆): 11.27 (1H, s), 10.18 (1H, s), 9.06 (1H, s), 8.14 (1H, s), 7.96 (2H, d, J=8.4 Hz), 7.85 (3H, m), 7.72 (2H, d, J=8.4 Hz), 7.43 (2H, m), 7.04 (2H, d, J=8.9 Hz), 4.11 (2H, q, J=6.9 Hz), 1.35 (3H, t, J=6.9 Hz). MS: 377.4 (M+1); 375.2 (M−1).

N-hydroxy-4-[3-(4-dimethylaminophenylcarbonylamino)phenyl]benzamide. ¹H NMR (DMSO-d₆): 11.27 (1H, s), 9.96 (1H, s), 8.14 (1H, s), 8.86 (5H, m), 7.72 (2H, d, J=8.2 Hz), 7.40 (2H, m), 6.76 (2H, d, J=8.9 Hz), 2.98 (6H, s). MS: 376.6 (M+1); 374.2 (M−1).

N-hydroxy-4-[3-(4-isopropylphenylcarbonylamino)phenyl]benzamide. ¹H NMR (DMSO-d₆): 11.26 (1H, s), 10.25 (1H, s), 8.14 (1H, s), 7.92-7.81 (5H, m), 7.70 (2H, d, J=8.4 Hz), 7.45-7.38 (4H, m), 2.97 (1H, M), 1.23 (6H, d, J=6.9 Hz). MS: 375.2 (M+1); 373.2 (M−1).

N-hydroxy-4-[3-(4-trifluoromethoxyphenylcarbonylaniino)phenyl]benzamide. ¹H NMR (DMSO-d₆): 11.27 (1H, s), 10.45 (1H, s), 9.05 (1H, s), 8.11 (3H, m), 7.87 (2H, d, J=8.4 Hz), 7.82 (1H, m), 7.73 (2H, d, J=8.4 Hz), 7.54 (2H, d, J=8.2 Hz), 7.47 (2H, m). MS: 417.2 (M+1); 415.4 (M−1).

N-hydroxy-4-[3-(3-fluoro-4-methoxyphenylcarbonylamino)phenyl]benzamide. ¹H NMR (DMSO-d₆): 11.26 (1H, s), 10.23 (1H, s), 8.12 (1H, s), 7.88-7.80 (5H, m), 7.72 (2H, d, J=8.4 Hz), 7.44 (2H, m), 7.32 (1H, t, J=8.6 Hz), 3.92 (3H, s). MS: 381.4 (M+1); 379.4 (M−1).

N-hydroxy-4-[3-(4-methoxy-2-methylphenylcarbonylamino)phenyl]benzamide. ¹H NMR (DMSO-d₆): 11.26 (1H, s), 10.24 (1H, s), 9.05 (1H, s), 8.11 (1H, s), 7.85 (2H, d, J=8.4 Hz), 7.75 (1H, m), 7.70 (2H, d, J=8.4 Hz). 7.50-7.39 (3H, m), 6.85 (2H, m), 3.79 (3H, s), 2.41 (3H, s). MS: 377.2 (M+1); 375.2 (M−1).

N-hydroxy-4-[3-(2,4-dimethoxyphenylcarbonylamino)phenyl]benzamide. ¹H NMR (DMSO-d₆): 11.27 (1H, s), 10.02 (1H, s), 9.05 (1H, s), 8.09 (1H, s), 7.86 (2H, d, J=8.2 Hz), 7.75 (4H, m), 7.42 (2H, m), 6.69 (2H, m), 3.97 (3H, s), 3.84 (3H, s). MS: 393.4 (M+1); 391.2 (M−1).

N-hydroxy-4-[3-(4-chloro-2-methoxyphenylcarbonylamino)phenyl]benzamide. ¹H NMR (DMSO-d₆): 11.27 (1H, s), 10.21 (1H, s), 9.05 (1H, s), 8.08 (1H, s), 7.85 (2H, d, J=8.2 Hz), 7.76-7.64 (4H, m), 7.45 (2H, m), 7.28 (1H, d, J=1.7 Hz), 7.13 (1H, dd, J=8.2 Hz, 1.7 Hz), 3.93 (3H, s). MS: 397.0 (M+1); 395.0 (M−1).

N-hydroxy-4-[3-(pyridin-4-ylcarbonylamino)phenyl]benzamide. ¹H NMR (DMSO-d₆): 11.27 (1H, s), 10.60 (1H, s), 9.06 (1H, s), 8.80 (2H, d, J=4.9 Hz), 8.13 (1H, s), 7.90-7.80 (5H, m), 7.72 (2H, d, J=8.2 Hz), 7.49 (2H, d, J=4.9 Hz). MS: 334.4 (M+1); 332.2 (M−1).

N-hydroxy-4-[3-(pyridin-3-ylcarbonylamino)phenyl]benzamide. ¹H NMR (DMSO-d₆): 11.28 (1H, s), 10.54 (1H, s), 9.13 (1H, s), 9.06 (1H, s), 8.77 (1H, d, J=3.5 Hz), 8.32 (1H, d, J=7.9 Hz), 8.14 (1H, s), 7.87 (2H, d, J=8.4 Hz), 7.82 (1H, m), 7.72 (2H, d, J=8.4 Hz), 7.59 (1H, m), 7.48 (2H, d, J=5.0 Hz). MS: 334.4 (M+1); 332.2 (M−1).

N-hydroxy-4-[3-(2-methylphenylcarbonylamino)phenyl]benzamide. ¹H NMR (DMSO-d₆): 11.26 (1H, s), 10.39 (1H, s), 9.05 (1H, br s), 8.12 (1H, s), 7.85 (2H, d, J=8.2 Hz), 7.74 (1H, m), 7.70 (2H, d, J=8.2 Hz), 7.50-7.30 (6H, m), 2.39 (3H, s). MS: 347.2 (M+1); 345.2 (M−1).

N-hydroxy-4-[3-(2,4-dimethylphenylcarbonylamino)phenyl]benzamide. ¹H NMR (DMSO-d₆): 11.27 (1H, s), 10.31 (1H, s), 9.07 (1H, br s), 8.12 (1H, s), 7.86 (2H, d, J=8.4 Hz), 7.75 (1H, m), 7.69 (2H, d, J=8.4 Hz), 7.42 (3H, m), 7.12 (2H, m), 2.37 (3H, s), 2.32 (3H, s). MS: 361.4 (M+1); 359.2 (M−1).

N-hydroxy-4-[3-(2,5-dimethylphenylcarbonylamino)phenyl]benzamide. ¹H NMR (DMSO-d₆): 11.27 (1H, s), 10.36 (1H, s), 9.06 (1H, br s), 8.12 (1H, s), 7.85 (2H, d, J=8.4 Hz), 7.75 (1H, m), 7.69 (2H, d, J=8.4 Hz), 7.42 (2H, m), 7.30 (1H, s), 7.19 (2H, s), 2.34 (3H, s), 2.32 (3H, s). MS: 361.4 (M+1); 359.2 (M−1).

N-hydroxy-4-[3-(2-methylthiophen-2-ylcarbonylamino)phenyl]benzamide. ¹H NMR (DMSO-d₆): 11.27 (1H, s), 10.19 (1H, s), 9.07 (1H, br s), 8.07 (1H, s), 7.85 (3H, m), 7.73 (3H, m), 7.42 (2H, m), 6.92 (1H, d, J=3.7 Hz), 2.49 (3H, s). MS: 353.6 (M+1); 351.2 (M−1).

### Example 4

### Synthesis of N-hydroxy-4-[3-(N-phenylaminocarbonyl)phenyl]benzamide

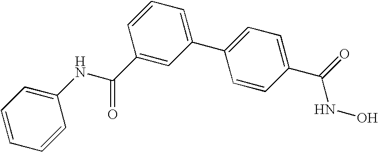

Step 1

To a prestirred (30 min.) solution of 3-bromobenzoic acid (1.0 g, 5.0 mmol) and tetrakis(triphenylphosphine)palladium(0) (0.29 g, 0.25 mmol) in toluene (30 ml) and ethanol (7 ml) was added 4-methoxycarbonylphenylboronic acid (1.0 g, 5.5 mmol) and K₂CO₃ (3.5 g, 25 mmol). The resulting slurry was heated to 75° C. for 3 hr then cooled to room temperature and diluted with H₂O (200 ml) and cooled to 0° C. and then acidified to pH=2 (300 ml). The aqueous layer was extracted with ethyl acetate (200 ml), then dried (MgSO₄), filtered and concentrated to collect a 1.68 g of methyl 4-(3-carboxyphenyl)benzoate as a light green solid.

Step 2

To a solution of methyl 4-(3-carboxyphenyl)benzoate (0.25 g, 1.0 mmol), aniline (0.1 ml, 1.0 mmol) and TEA (0.7 ml) in DMF (7 ml) was added BOP-CL (0.47 g, 1.1 mmol). After stirring 12 hr at room temperature, the solution was diluted with ethyl acetate (300 ml) and washed with 1N HCl (2×200 ml), then mild NaHCO₃ (2×200 ml). The organic layer was dried (MgSO₄), filtered and concentrated. Flash chromatography (25% ethyl acetate/hexanes) provided 0.18 g (56%) of methyl 4-[3-(phenylaminocarbonyl)phenyl]benzoate as a white solid. ¹H NMR (DMSO-d₆): 8.28 (1H, s), 8.07 (2H, d, J=8.4 Hz), 7.95 (4H, m), 7.77 (2H, d. J=7.7 Hz), 7.65 (1H, t, J=7.7 Hz), 7.36 (2H, t, J=7.7 Hz), 7.10 (1H, t, J=7.4 Hz), 3.87 (3H, s). MS: 332.2 (M+1); 330.4 (M−1).

Step 3

A solution of methyl 4-[3-(phenylaminocarbonyl)phenyl]benzoate(0.18 g, 0.54 mmol), NaOH (0.5 g dissolved in 5 ml H₂O), THF (10 ml) and MeOH (10 ml) was stirred for 8 hr at room temperature. The solution was then acidified with 1N HCl (100 ml) and extracted into ethyl acetate (100 ml). The organic layer was dried (MgSO₄), filtered and concentrated to collect 0.18 g (93%) of crude 4-[3-(phenylaminocarbonyl)phenyl]benzoic acid. To the crude acid stirred in THF (8 ml) and TEA (0.15 ml) was added thionyl chloride (0.1 ml, 1.2 mmol). After stirring 1 hr at room temperature, the solvent was removed in vacuo and then the resulting oil was dried under high vacuum. The oil was dissolved in THF (10 ml), cooled with an ice bath and then 50% aq. hydroxylamine (3 ml) was added. The cooling bath was removed and the light orange solution was stirred 30 minutes at room temperature. The reaction solution was then diluted with 1N HCl (100 ml) and extracted with ethyl acetate (100 ml). The solvent was dried (MgSO4), filtered and concentrated to collect 0.11 g (61%) of the title compound as an off-white solid; ¹H NMR (DMSO-d₆): 11.31 (1H, s), 10.35 (1H, s), 8.26 (1H, s) 7.95-7.82 (6H, m), 7.77 (2H, d. J=7.7 Hz), 7.64 (1H, t, J=7.7 Hz), 7.38 (2H, t, J=7.7 Hz), 7.16 (1H, t, J=7.4 Hz), MS: 333.0 (M+1); 331.4 (M−1).

Following the procedure described in Example 4 above, using different starting materials, the following compounds of Formula I were prepared:

N-hydroxy-4-[3-(N-benzylaminocarbonyl)phenyl]benzamide: ¹H NMR (DMSO-d₆): 11.29 (1H, s), 9.19 (1H, t, J=5.9 Hz), 9.07 (1H, s), 8.24 (1H, s), 7.94-7.81 (5H, m), 7.59 (1H, t, J=7.7 Hz), 7.34-7.23 (4H, m), 4.52 (2H, d, J=5.9 Hz). MS: 347.2 (M+1); 345.2 (M−1).

N-hydroxy-4-[3-(N-2,2,2-trifluoroethylaminocarbonyl)phenyl]benzamide: ¹H NMR (DMSO-d₆): 9.23 (1H, t, J=5.9 Hz), 9.07 (1H, s), 8.21 (1H, s), 7.93-7.80 (6H, m), 7.60 (1H, t, J=7.7 Hz), 4,12 (2H, m). MS: 339.4 (M+1); 337.4 (M−1).

N-hydroxy-4-[3-N-3-methylbutylaminocarbonyl)phenyl]benzamide: ¹H NMR (DMSO-d₆): 11.23 (1H, s), 9.01 (1H, s), 8.49 (1H, t, J=5.5 Hz), 8.09 (1H, s), 7.84-7.74 (6H, m), 7.50 (1H, t, J=7.7 Hz), 3.25 (2H, m)1.55 (1H, m), 1.39 (2H, q, J=6.9 Hz), 0.85 (6H, d, J=6.7 Hz). MS: 327.2 (M+1); 325.4 (M−1).

N-hydroxy-4-[3-(N-methylaminocarbonyl)phenyl]benzamide: ¹H NMR (DMSO-d₆): 11.29 (1H, s), 9.06 (1H, s), 8.57 (1H, t, J=4.4 Hz),8.14 (1H, s), 7.89-7.80 (6H, m),7.56 (1H, t, J=7.9 Hz), 2.80 (3H, d, J=4.4 Hz). MS: 271.4 (M+1); 269.0 (M−1).

### Example 5

### Synthesis of N-hydroxy-4-(4,5-diphenylimidazol-2-yl)benzamide

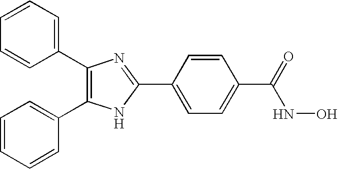

Step 1

A solution of benzil (1.05 g, 5.0 mmol), methyl 4-formylbenzoate (0.9 g, 5.5 mmol) and ammonium acetate (3.9 g, 50 mmol) was heated to 75° C. for 18 hr in glacial acetic acid (30 ml). The yellow slurry was then cooled to room temperature and diluted with ethyl acetate (200 ml) and washed with water (3×200 ml) then sat. NaHCO₃ (200 ml). The organic layer was dried (MgSO₄), filtered and concentrated to collect 1.8 g (˜100%) of a light yellow solid. The solid was then stirred in methanol (20 ml) and to this was added NaOH (1.1 g in 10 ml of water). After stirring 2 hr at room temperture, water was added (200 ml) and the pH of the solution was adjusted to ˜5 with 1N HCl. The aqueous layer was extracted with ethyl acetate (200 ml) then the organic layer was dried (MgSO₄), filtered and concentrated to collect 0.93 g (97%) of 4-(4,5-diphenyl-1H-imidazole-2-yl)-benzoic acid as a light yellow solid. ¹H NMR (DMSO-d₆): 8.18 (2H, d, J=8.4 Hz), 8.04 (2H, d, J=8.4 Hz), 7.53 (m, 4H), 7.40-7.22 (m, 6H). MS: 341.4 (M+1); 339.6 (M−1).

Step 2

4-(4,5-Diphenyl-1H-benzimidazol-2-yl)-benzoic acid was converted to the title compound by proceeding as described in Example 1. ¹H NMR (DMSO-d₆): 11.21 (1H, s), 9.02 (1H, br s), 8.08 (2H, d, J=8.4 Hz), 7.79 (2H, d, J=8.4 Hz), 7.48 (4H, m), 7.35-7.22 (6H, m). MS: 356.6 (M+1); 354.4 (M−1).

### Example 6

### Synthesis of N-hydroxy-4-(1H-benzimidazol-2-yl)benzamide

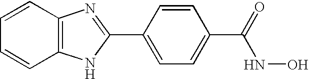 Step 1

A solution of 1,2-phenylenediamine (0.5 g, 4.6 mmol), methyl 4-formylbenzoate (0.76 g, 4.6 mmol) and sodium metabisulfite (1.3 g, 6.9 mmol) was heated to reflux for 2 hr. The solution was cooled to room temperature and diluted with ethyl acetate (150 ml) then washed with mild NaHCO₃ (200 ml). The organic layer was dried (MgSO₄), filtered and concentrated to collect 1.14 g (98%) of a tan solid. The tan solid was then stirred in methanol (20 ml) and to this was added NaOH (0.95 g in 5 ml of water). After stirring overnight at room temperature, water was added (200 ml) and the pH of the solution was adjusted to 5 with 1N HCl. The aqueous layer was extracted with ethyl acetate (200 ml) then dried (MgSO₄), filtered and concentrated to collect 0.42 g (74%) of 4-(1H-benzoimidazole-2-yl)-benzoic acid as a light yellow solid. ¹H NMR (DMSO-d₆): 8.28 (2H, d, J=8.4 Hz), 8.09 (2H, d, J=8.4 Hz), 7.62 (2H, m), 7.23 (2H, m). MS: 239.2 (M+1); 237.0 (M−1).

Step 2

4-(1H-imidazole-2-yl)-benzoic acid was converted to the title compound by proceeding as described in Example 1. ¹H NMR (DMSO-d₆): 11.29 (1H, s), 8.18 (2H, d, J=8.4 Hz), 7.87 (2H, d, J=8.4 Hz), 7.58 (2H, m), 7.20 (2H, m). MS: 254.4 (M+1); 252.2 (M−1).

Proceeding a described in Example 6 above, but using suitable starting materials provided the following compounds of Formula I:

N-Hydroxy-4-(1-methyl-1H-benzimidazol-2-yl)-benzamide: ¹H NMR (DMSO-d₆): 11.34 (1H, s), 9.12 (1H, br s), 7.89 (4H, s), 7.63 (2H, m), 7.25 (2H, m), 3.85 (3H, s). MS: 268.4 (M+1); 266.2 (M−1).

N-hydroxy-4(4-benzyloxy-1H-benzimidazol-2-yl)-benzamide: ¹H NMR (DMSO-d₆): 11.26 (1H, s), 9.06 (1H, s), 8.27 (1H, d, J=8.4 Hz), 8.15 (2H, d, J=8.4 Hz), 7.83 (3H, m), 7.50 (2H, m), 7.33 (3H, m), 7.06 (1H, m), 5.30 (2H, s). MS: 360.0 (M+1); 358.2 (M−1).

### Example 7

### Synthesis of N-hydroxy-4-(3-benzyloxyphenyl)benzamide

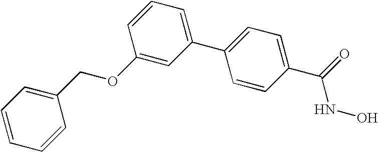 Step 1

To a prestirred (30 min) solution of 3-bromophenol (0.52 g, 3.0 mmol) and tetrakis(triphenylphosphine)palladium(0) (0.18 g, 0.15 mml) in toluene (20 ml) and methanol (5 ml) was added 4-methoxycarbonyl phenylboronic acid (0.6 g, 3.3 mmol) and K₂CO₃ (2.1 g, 15 mmol). The resulting slurry was heated to 75° C. for 3 hr then cooled to room temperature and diluted with ethyl acetate (100 ml) and washed with 1N HCl (300 ml). The organic layer was dried (MgSO₄), filtered and concentrated to a brown oil. Flash chromatography (25% ethyl acetate/hexanes) provided 0.52 g (75%) of methyl 4-(3-hydroxyphenyl)benzoate as a tan solid.

Step 2

A slurry of methyl 4-(3-hydroxyphenyl)benzoate (0.17 g, 0.74 mmol), benzyl bromide (0.1 ml, 0.82 mmol) and K₂CO₃ (0.51 g, 3.7 mmol) in acetonitrile (7 ml) was strirred for 18 hr at room temperature. The slurry was then diluted with ethyl acetate (100 ml) and washed with water (200 ml). The organic layer was dried (MgSO₄), filtered and concentrated to collect a tan solid. Flash chromatography (10% ethyl acetate/hexanes) provided 0.26 g (˜100%) of methyl 4-(3-benzyloxyphenyl)benzoate as a white solid.

Step 3

The title compound was obtained by following the procedure described in Example 1, steps 2 and 3 above. ¹H NMR (DMSO-d₆): 11.27 (1H, s), 9.06 (1H, s), 7.83 (2H, d, J=8.4 Hz), 7.75 (2H, d, J=8.4 Hz), 7.55-7.28 (8H, m), 7.05 (1H, m), 5.20 (2H, s). MS: 320.0 (M+1); 318.4 (M−1).

Proceeding a described in Example 7 above, but using suitable starting materials provided N-hydroxy-4-[3-(3-methoxybenzyloxy)phenyl]benzamide: ¹H NMR (DMSO-d₆): 11.28 (1H, s), 7,84 (2H, d, J=8.4 Hz), 7.75 (2H, d, J=8.4 Hz), 7.42-7.27 (4H, m), 7.04 (3H, m), 6.89 (1H, m), 5.17 (2H, s), 3.76 (3H, s). MS: 350.2 (M+1); 348.0 (M−1).

### Example 8

### Synthesis of N-hydroxy-4-(3-aminophenyl)benzamide

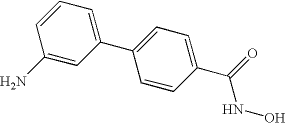

N-hydroxy-4-(3-aminophenyl)benzamide was prepared by using the method of Example 3, Step 3 using N-hydroxy-4-(3-nitrophenyl)benzamide as the starting material. ¹H NMR (DMSO-d₆): 9.03 (1H, br s), 7.80 (2H, d, J=8.2 Hz), 7.60 (2H, d, J=8.2 Hz), 7.10 (1H, t, J=7.7 Hz), 6.86 (1H, t, J=1.7 Hz), 6.80 (1H, d, J=7.7 Hz), 6.55 (1H, dd, J=7.7 Hz, 1.7 Hz), 5.18 (2H, br s). MS: 229.6 (M+1); 227.4 (M−1).

### Example 9

### Synthesis of N-hydroxy-4-[3-(4-methoxyphenylcarbonylamino)phenyl]benzamide

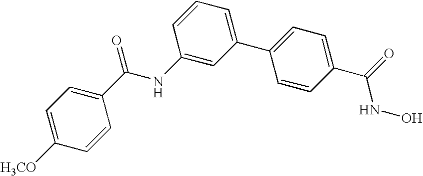

Step 1

To a solution of ethyl 4-(3-aminophenyl)benzoate (0.2 g, 0.83 mmol) and TEA (4.1 mmol, 0.6 mml) in THF (7 ml) was added anisoyl chloride (0.14 g, 0.83 mmol). After stirring 1 hr at room temperature, the mixture was diluted with ethyl acetate (50 ml), and washed with 1 N HCl (100 ml). The organic layer was dried (MgSO₄), filtered and concentrated to collect 0.4 g of a light brown oil. The oil was stirred in THF (5 ml) and methanol (5 ml) and to this was added KOH (0.7 g in 3 ml of H₂O). After stirring 2 hr at room temperature, the solution was diluted with H₂O (100 ml) and acidified with 1 N HCl (pH=˜2). The aqueous layer was extracted with ethyl acetate (100 ml), and then the organic layer was dried (MgSO₄), filtered and concentrated to collect 0.29 g (˜100%) of 4-[3-(4-methoxyphenylcarbonylamino)phenyl]benzoic acid as a white solid.

4-[3-(4-methoxyphenylcarbonylamino)phenyl]benzoic acid was converted to the title compound by proceeding as described in Example 1 above, but with the substitution of oxalyl chloride with thionyl chloride and the omission of DMF.

Proceeding a described in Example 9 above, but using suitable starting materials N-hydroxy-4-[3-(isopropylcarbonylamino)phenyl]benzamide. ¹H NMR (DMSO-d₆): 11.26 (1H, s), 9.93 (1H, s), 9.05 (1H, s), 7.99 (1H, s), 7.84 (2H, d, J=8.4 Hz), 7.68 (2H, d, J=8.4 Hz), 7.37 (2H, m), 2.60 (1H, m), 1.10 (6H, d, J=6.7 Hz); MS: 297.4 (M−1), 299.6 (M+1)

### Example 10

### Synthesis of N-hydroxy-4-(3-N,N-dimethylaminocarbonylphenyl)benzamide

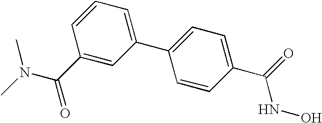 Step 1

To a prestirred (30 min.) solution of 3-bromomethyl benzoate (3.9 g, 18 mmol) and tetrakis(triphenylphosphine)palladium(0) (0.63 g, 0.54 mmol) in toluene (70 ml) and methanol (20 ml) was added 4-carboxyphenyl boronic acid (3 g, 18 mmol) and K₂CO₃ (12.5 g, 90 mmol). The resulting slurry was heated to 75° C. for 3 hr then cooled to room temperature and slowly acidified to pH=˜2 with 1N HCl. The mixture was then extracted with ethyl acetate (200 ml). The organic layer was washed with brine (200 ml), then dried (MgSO₄), filtered and concentrated to collect 5.1 g (˜100%) of methyl 4-(3-carboxyphenyl)benzoate as a white solid.

Step 2

To a solution of methyl 4-(3-carboxyphenyl)benzoate (0.5 g, 1.9 mmol), O-(tetrahydro-2H-pyran-2-yl)-hydroxylamine (0.27 g, 2.3 mmol), HOBt (0.26 g, 1.9 mmol) and diisopropylethylamine (1.7 ml, 9.8 mmol) in DMF (15 ml) was added EDC (0.64 g, 3.3 mmol). After 24 hr at room temperature, the solution was diluted with ethyl acetate (100 ml) and carefully washed with 0.5N HCl (200 ml), then sat. NaHCO₃ (200 ml). The organic layer was dried (MgSO₄), filtered and concentrated to collect 0.72 g (−100%) of a white solid/oil. To the crude methyl ester in THF (10 ml) and methanol (10 ml) was added KOH (0.6 g in 2 ml H₂O). After stirring 12 hr at room temperature, most of the solvent was evaporated and then the remaining oil was partitioned between ethyl acetate (100 ml) and H₂O (100 ml adjusted to pH=˜5). The layers were separated and the organic layer was dried (MgSO₄), filtered and concentrated to collect 0.60 g (90%) of N-(tetrahydropyran-2-yloxy)-4-(3-carboxyphenyl)benzamide as a white solid.

Step 3

To a solution of N-(tetrahydropyran-2-yloxy)-4-(3-carboxyphenyl)benzamide (0.1 g, 0.29 mmol), dimethylamine hydrochloride (0.12 g, 1.5 mmol) and TEA (0.3 ml, 2.1 mmol) in DMF was added BOP-Cl (0.14 g, 3.2 mmol). After strirring 18 hr at room temperature, the solution was diluted with ethyl acetate (100 ml) and washed with sat. NaHCO₃ (100 ml) and then 1N HCl (100 ml). The organic layer was dried (MgSO₄), filtered and concentrated to collect a colorless oil. The oil was stirred in methanol (2 ml) and to this was added 4M HCl/dioxane (0.25 ml). After 30 min at room temperature the solvent was evaporated and the remaining residue was dissolved in THF (2 ml) and precipitated into ether (50 ml). The resulting tan solid was collected by filtration and dried under high vacuum to provide 50 mg (60%) of the title compound as a tan solid. ¹H NMR (DMSO-d₆): 11.27 (1H, br s), 9.04 (1H, s), 7.81 (5H, m), 7.69 (1H, s), 7.53 (1H, t, J=7.7 Hz), 7.39 (1H, d, J=7.4 Hz), 2.99 (3H, s), 2.93 (3H, s); MS: 283.5 (M−1), 285.4 (M+1)

Proceeding a described in Example 10 above, but using suitable starting materials the following compounds of Formula I were prepared:

N-hydroxy-4-(3-morpholin-4-ylcarbonylphenyl)benzamide

¹H NMR (DMSO-d₆) 11.27 (1H, br s), 9.06 (1H, s), 7.81 (5H, m), 7.71 (1H, s), 7.55 (1H, t, J=7.4 Hz), 7.41 (1H, d, J=7.4 Hz), 3.62 (8H, br s); MS: 325.4 (M−1), 327.4 (M+1).

N-hydroxy-4-[3-(N-methyl-N-phenylaminocarbonyl)phenyl]benzamide. ¹H NMR (DMSO-d₆) 11.18 (1H, br s), 8.25 (1H, s), 7.73 (2H, d, J=8.4 Hz), 7.54 (1H, d, J=6.9 Hz), 7.45 (2H, d, J=8.4 Hz), 7.2 (8H, m), 3.34 (3H, s); MS: 345.2 (M−1), 347.2 (M+1).

N-hydroxy-4-[3-(N-4-chlorophenylaminocarbonyl)phenyl]benzamide. ¹NMR (DMSO-d₆): 11.31 (1H, br s), 10.49 (1H, s), 10.19 (1H, br s), 8.26 (1H, s), 7.88 (8H, m), 7.64 (1H, t, J=8.4 Hz), 7.42 (2H, d, J=8.7 Hz); MS: 365.2 (M−1), 367.2 (M+1).

N-hydroxy-4-(3-[N-2-(4-methoxyphenyl)ethylaminocarbonyl]phenyl}benzamide. ¹H NMR (DMSO-d₆) 11.29 (1H, s), 9.07 (1H, br s), 8.67 (1H, t, J=5.6 Hz), 8.12 (1H, s), 7.85 (6H, m), 7.56 (1H, t, J=7.8 Hz), 7.16 (2H, d, J=8.5 Hz), 6.85 (2H, d, J=8.5 Hz), 3.69 (3H, s), 3.46 (2H, dd, J=6.2 Hz, 12.6 Hz), 2.79 (2H, t, J=7.9 Hz); MS: 389.2 (M−1), 390.16 (M+1).

N-hydroxy-4-[3-(N-4-chlorobenzylaminocarbonyl]phenyl]benzamide. ¹H NMR (DMSO-d₆) 9.22 (1H, t, J=6.1 Hz), 7.86 (6H, m), 7.59 (1H, t, J=7.8 Hz), 7.49 (1H, s), 7.37 (4H, m), 4.49 (2H, d, J=5.9 Hz); MS: 379.4 (M−1), 381.4 (M+1).

N-hydroxy-4-{3-[N-2-(phenyl)ethylaminocarbonyl]phenyl}benzamide.

¹H NMR (DMSO-d₆): 11.29 (1H, br s), 9.06 (1H, s), 8.71 (1H, t, d=5.6 Hz), 8.12 (1H, s), 7.84 (6H, m), 7.56 (1H, t, J=7.7 Hz),7.25 (5H, m), 3.5 (2H, dd, J=12.6 Hz, 6.5 Hz), 2.86 (2H, t, J=6.5 Hz); MS: 359.2 (M−1), 361.4 (M+1).

N-hydroxy-4-[3-(N-2-hydroxyethylaminocarbonyl)phenyl]benzamide. ¹H NMR (DMSO-d₆): 8.60 (1H, br s), 8.18 (1H, s), 7.86 (6H, m), 7.56 (1H, t, J=7.7 Hz), 3.52 (2H, t, J=6.2 Hz), 3.35 (2H, m); MS: 299.4 (M−1), 301.4 (M+1).

N-hydroxy-4-[3-(piperidin-1-ylcarbonyl)phenyl]benzamide. ¹H NMR (DMSO-d₆): 7.82 (2H, d, J=8.6 Hz), 7.72 (3H, d, J=8.6 Hz), 7.61 (1H, s), 7.48 (1H, t, J=7.7 Hz), 7.31 (1H, d, J=7.7 Hz), 3.24 (4H, m), 1.42 (6H, m); MS: 323.4 (M−1), 325.6 (M+1).

### Example 11

### Synthesis of N-hydroxy-4-[3-(N,N-dimethylaminocarbonylmethyl)phenyl]benzamide

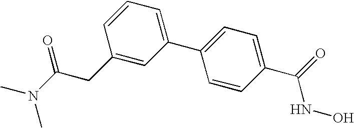 Step 1

Proceeding as described in Example 10, Step 1 above, but with the substitution of 3-bromomethyl benzoate with methyl 3-bromophenylacetate provided 4-[3-methoxycarbonylmethyl)phenyl]benzoic acid which was then converted to N-(tetrahydropyran-2-yloxy)-4-(3-carboxymethylphenyl)benzamide utilizing the procedure described in Example 10, Step 2 above.

Step 2

N-(Tetrahydropyran-2-yloxy)-4-(3-carboxymethylphenyl)benzamide was converted to the title compound by proceeding as described in Example 10, Step 3. ¹H NMR (DMSO-d₆): 11.26 (1H, br s), 7.84 (2H, d, J=8.3 Hz), 7.71 (2H, d, J=8.3 Hz), 7.56 (2H, m), 7.40 (1H, t, J=7.7 Hz), 3.76 (2H, s), 3.03 (3H, s), 2.83 (3H, s); MS: 297.4 (M−1), 299.2 (M+1).

Proceeding a described in Example 11 above, but using suitable starting materials the following compounds of Formula I were prepared:

N-hydroxy-4-{3-[N-2-(4-methoxyphenyl)ethylaminocarbonylmethyl]phenyl)benzamide. ¹H NMR (DMSO-d₆): 11.30 (1H, br s), 9.06 (1H, br s), 8.09 (1H, t, J=6.8 Hz), 7.85 (2H, d, J=8.4 Hz), 7.71 (2H, d, J=8.4 Hz). 7.57 (3H, d. J=7.7 Hz). 7.39 (1H, t, J=7.7 Hz), 7.24 (1H, d, J=7.7 Hz), 7.04 (2H, d, J=8.7 Hz), 6.76 (2H, d, J=8.7 Hz), 3.66 (3H, s), 3.45 (2H, s), 3.22 (2H, dd, J=12.9 Hz, 6.8 Hz), 2.61 (2H, t, J=6.8 Hz); MS: 403.4 (M−1), 405.2 (M+1).

N-hydroxy-4-[3-(N-4-chlorobenzylaminocarbonylmethyl)phenyl]benzamide. ¹H NMR (DMSO-d₆): 11.26 (1H, br s), 9.05 (1H, br s), 8.61 (1H, t, J=5.8 Hz), 7.84 (2H, d, J=8.4 Hz), 7.68 (2H, d, J=8.4 Hz), 7.57 (2H, d, J=7.4 Hz), 7.40 (1H, t, J=7.4 Hz), 7.35 (1H, s), 7.26 (4H, m), 4.24 (2H, d, J=5.8 Hz), 3.54 (2H, s); MS: 393.2 (M−1), 395.2 (M+1).

N-hydroxy-{3-[N-2-(phenyl)ethylaminocarbonylmethyl]phenyl}benzamide. ¹H NMR (DMSO-d₆): 11.22 (1H, br s), 8.07 (1H, t, J=5.32 Hz), 7.79 (2H, d, J=8.4 Hz), 7.65 (2H, d, J=8.4 Hz), 7.50 (2H, m), 7.34 (1H, t, J=7.7 Hz), 7.13 (6H, m), 3.29 (4H, m), 2.63 (2H, t, J=7.2 Hz); MS: 373.2 (M−1), 375.2 (M+1).

N-hydroxy-4-[3-(morpholin-4-ylcarbonylmethyl)phenyl]benzamide. ¹H NMR (DMSO-d₆): 11.26 (1H, br s), 7.83 (2H, d, J=8.4 Hz), 7.71 (2H, d, J=8.4 Hz), 7.57 (2H, d, J=7.6 Hz), 7.41 (1H, t, J=7.6 Hz), 3.79 (2H, s), 3.46 (8H, m); MS: 339.4 (M−1), 341.4 (M+1).

N-hydroxy-4-[3-(N-methyl-N-phenylaminocarbonylmethyl)phenyl]benzamide. ¹H NMR (DMSO-d₆): 11.20 (1H, br s), 7.78 (2H, d, J=8.4 Hz), 7.61 (2H, d, J=8.4 Hz), 7.44 (4H, m), 7.3 (5H, m), 3.39 (2H, s), 3.13 (3H, s); MS: 359.2 (M−1), 361.4 (M+1).

N-hydroxy-3′-(4-chlorophenylcarbamoylmethylphenyl)benzamide. ¹H NMR (DMSO-d₆): 11.21 (1H, s), 10.27 (1H, s), 8.99 (1H, s), 7.79 (2H, d, J=8.2 Hz), 7.67 (2H, d, J=8.16 Hz), 7.57 (3H, m), 7.38 (1H, t, J=7.67 Hz), 7.28 (4H, m), 3.67 (2H, s); MS: 379.4 (M−1), 381.4 (M+1).

### Example 12

### Synthesis of N-hydroxy-4-(6-fluoro-4-phenoxy-1H-benzoimidazol-2-yl)-benzamide

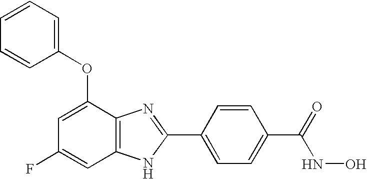 Step 1

To a solution of 1,3,5-trifluoro-2-nitrobenzene (2.2 ml, 19 mmol) in THF 75 ml was added ammonium hydroxide (2 ml). After stirring 16 hr at room temperature, the THF was evaporated and the residue was diluted wuth ethyl acetate (250 ml) and washed with brine (300 ml). The organic layer was dried (MgSO₄), filtered and concentrated to collect 3.2 g (−100%) of 2-nitro-3,5-difluorophenylamine as an orange solid.

Step 2

To a prestirred (25 min. at room temperature) slurry of phenol (0.26 g, 2.7 mmol) and sodium hydride (70 mg, 2.7 mmol) in THF (14 ml) was added 2-nitro-3,5-difluorophenylamine (0.43 g, 2.5 mmol) dissolved in THF (2 ml). After stirring 8 hr at room temperature, the brown solution was diluted with ethyl acetate (100 ml) and washed with H₂O (100 ml) and then brine (100 ml). The organic layer was dried (MgSO₄), filtered and concentrated to collect 0.52 g (84%) of 5-fluoro-2-nitro-3-phenoxyphenylamine as an orange solid.

Step 3

A solution of 5-fluoro-2-nitro-3-phenoxyphenylamine (0.52 g, 2.1 mmol) and palladium hydroxide (70 mg) in methanol (30 ml) was stirred at room temperature and subjected to atmospheric hydrogen for 4 hr. The catalyst was then removed by filtering the reaction slurry through celite. The solvent was removed in vacuo to collect 0.46 g of a green semi-solid. The resulting green solid was redissolved in methanol (20 ml) and to this was added methyl 4-formyl benzoate (0.35 g, 2.1 mmol) and sodium metabisulfite (0.6 g, 3.1 mmol). The solution was refluxed for 45 min, then cooled to room temperature and diluted with ethyl acetate (150 ml) and washed with sat. NaHCO₃ (200 ml). The organic layer was dried (MgSO₄), filtered and concentrated to collect a brown solid. Flash chromatography (25% ethyl acetate/hexanes) provided 0.51 g (68%) of 4-(4-benzyloxy-6-fluoro-1H-benzoimidazol-2-yl)benzoic acid methyl ester as a light orange solid.

Step 4

To a solution of 4-(4-benzyloxy-6-fluoro-1H-benzoimidazol-2-yl)-benzoic acid methyl ester (0.34 g, 0.94 mmol) in THF (5 ml) and methanol (10 ml) was added KOH (0.26 g in 2 ml of H₂O). After 4 hr at room temperature, the pH of the solution was adjusted to ˜5 and then it was extracted with ethyl acetate (100 ml). The organic layer was dried (MgSO₄), filtered and concentrated to collect a brown solid. The resulting crude acid (0.23 g, 0.66 mmol) was stirred in THF (10 ml) at room temperature and to this was added TEA (0.46 ml, 3.3 mmol) and thionyl chloride (0.06 ml, 0.86 mmol). After 4 hr at room temperature, the THF was removed in vacuo, and the resulting residue was dried under high vacuum. The residue was stirred in THF (3 ml) and to this was added hydroxylamine (1 ml, 50% aq. solution). After stirring 30 min at room temperature, the solution was diluted with ethyl acetate (50 ml) and washed with H₂O (100 ml). The organic layer was dried (MgSO₄), filtered and concentrated to collect a brown solid. The solid was dissolved in THF (2 ml) and precipitated into ethyl acetate/ether (20 ml/20 ml). The resulting tan solid was collected by filtration to provide 0.15 g (63%) of N-hydroxy-4-(6-fluoro-4-phenoxy-1H-benzoimidazol-2-yl) benzamide. ¹H NMR (DMSO-d₆): 9.13 (1H, br s), 8.24 (2H, d, J=8.4 Hz), 7.89 (2H, d J=8.4 Hz), 7.26 (6H, m), 6.47 (1H, dd, J=11.1 Hz, 2.2 Hz); MS: 362.2 (M−1), 364.0 (M+1).

Proceeding a described in Example 12 above, but using suitable starting materials the following compounds of Formula I were prepared:

N-hydroxy-4-(6-fluoro-4-phenethyloxy-1H-benzoimidazol-2-yl)-benzamide. ¹HNMR (DMSO-d₆): 11.31 (1H, br s), 9.11 (1H, br s), 8.08 (4H, m), 7.3 (5H, m), 6.89 (1H, dd, J=12.1 Hz, 2.2 Hz), 6.67 (1H, dd, J=12.1 Hz, 2.2 Hz), 4.46 (2H, m), 3.15 (2H, m); MS: 390.4 (M−1), 392.2 (M+1).

N-hydroxy-4-[6-fluoro-4-(4-methoxy-phenoxy)-1H-benzoimidazol-2-yl]-benzamide. ¹H NMR (DMSO-d₆): 9.12 (1H, br s), 8.04 (4H, m), 7.03 (5H, m), 6.27 (1H, dd, J=10.8 Hz, 2.2 Hz), 3.76 (3H, s); MS: 392.4 (M−1), 394.2 (M+1).

N-hydroxy-4-[6-fluoro-4-(tetrahydro-furan-2-ylmethoxy)-1H-benzoimidazol-2-yl]-benzamide. ¹H NMR (DMSO-d₆): 11.32 (1H, br s), 9.11 (1H, s), 8.23 (2H, m), 7.89 (2H, m), 6.85 (2H, m), 4.21 (3H, m), 3.76 (2H, m), 1.89 (4H, m); MS: 370.2 (M−1), 372.2 (M+1).

N-hydroxy-4-4-(6-fluoro-4-isobutoxy-1H-benzoimidazol-2-yl)-benzamide. ¹H NMR (DMSO-d₆): 11.39 (1H, br s), 9.12 (1H, s), 8.27 (2H, m), 7.89 (2H, m), 6.8 (2H, m), 3.97 (2H, m), 2.13 (1H, m), 1.04 (6H, m); MS: 342.2 (M−1), 343.8 (M+1).

N-hydroxy-4-(4-cyclohexyloxy-6-fluoro-1H-benzoimidazol-2-yl)-benzamide. ¹H NMR (DMSO-d₆): 8.22 (2H, m), 7.86 (2H, d, J=8.4 Hz), 6.83 (2H, m), 4.67 (1H, m), 1.43 (10H, m); MS: 368.2 (M−1), 370.0 (M+1).

### Example 13

### Synthesis of N-hydroxy-4-[5-(N,N-dimethylaminocarbonyl)-3-(phenylcarbonylamino)-phenyl]benzamide

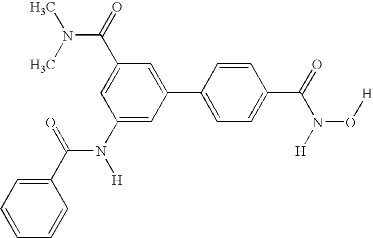 Step 1

To a solution of 3-nitrobenzoic acid (20.00 g, 119.67 mmol) in a mixture of trifluoroacetic acid (120 ml) and sulfuric acid (48 ml) was added N-bromosuccinimide (42.60 g, 239.35 mmol) protion wise. The reaction was stirred at room temperature under a nitrogen balloon overnight. The reaction was poured into ice and the resulting pale yellow precipitate was collected by vacuum filtration and air-dried to afford the 5-bromo-3-nitrobenzoic acid (28.0 g, 95% yield). ¹H NMR (DMSO-d₆) 8.61 (t, 1H, J=2.0 Hz), 8.53 (t, 1H, J=1.5 Hz), 8.39 (t, 1H, J=1.7 Hz); MS: calc 244.9; found 244.2 (M−1).

Step 2

To a solution of the 5-bromo-3-nitrobenzoic acid (10.00 g, 40.6 mmol) in methanol (250 ml) was added 4N hydrogen chloride in dioxane (25 ml) and the solution heated to reflux overnight. The reaction was concentrated to yield methyl 5-bromo-3-nitrobenzoate as a light cream-colored solid suitable for use in subsequent reactions (10.50 g, 99% yield).

Step 3

To a solution of the methyl 5-bromo-3-nitrobenzoate (5.00 g, 19.23 mmol) in a mixture of acetone (50 ml) and water (SOmI) was added 4-carboxyphenyl boronic acid (3.50 g, 21.15 mmol) followed by anhydrous potassium carbonate (6.65 g, 48.1 mmol). The reaction was flushed with nitrogen and to it was added palladium acetate (130 mg, 0.58 mmol). After stirring at room temperature for 30 minutes, the reaction was diluted with ethyl acetate and poured into a separatory funnel. The aqueous layer was drained and saved for later use. The organic layer was washed with 1N HCl and brine and set aside. The previous aqueous layer was placed into the separatory funnel and to it was added 1N HCl followed by water. The aqueous layer was extracted twice with ethyl acetate and the combined organic fractions washed with brine and dried over anhydrous sodium sulfate. Concentration afforded methyl 4-(3-nitro-5-ethoxy-carbonylphenyl)benzoic acid as a beige solid (5.80 g, 100% yield) suitably pure for use in the subsequent solid phase reactions.

Step 4

Hydroxylamine Wang resin (0.500 g, 1.04 mmol/g, 0.52 mmol) was pre-swelled with methylene chloride for 30 minutes. The solvent was drained and the resin washed with DMF. To the resin was added a solution of afforded methyl 4-(3-nitro-5-ethoxy-carbonylphenyl)benzoic acid in DMF (5 ml), HATU (0.395 g, 1.04 mmol), and DIEA (0.36 ml, 2.08 mmol) and the mixture agitated at room temperature overnight. The solvents were drained and the resin washed with DMF (4×) to provide resin bound 4-(3-nitro-5-ethoxycarbonylphenyl)benzamide.

Step 5

To the resin bound 4-(3-nitro-5-ethoxycarbonylphenyl)benzamide (0.52 mmol) was added tin (II) chloride dihydrate (1.17 g, 5.2 mmol) and DMF (5 ml) and the reaction agitated at room temperature overnight. The solvents were drained and the resin washed with DMF (4×), DCM (2×) and DMF (2×). A small amount of resin was cleaved with a solution of 50% TFA/49% DCM/1% water and the supernatant analyzed via HPLC and LCMS to monitor the conversion of 4-(3-nitro-5-ethoxycarbonylphenyl)benzoate to 4-(3-amino-5-ethoxycarbonylphenyl)benzamide.

Step 6A

To the resin bound 4-(3-amino-5-ethoxycarbonylphenyl)benzamide (0.52 mmol) was added DMF (5 ml), followed by the benzoic acid (2.08 mmol), HATU (0.79 g, 2.08 mmol), and DIEA (0.73 ml, 4.16 mmol) and the mixture agitated at room temperature overnight. The solvents were drained and the resin washed with DMF (4×). A small amount of resin was cleaved with a solution of 50% TFA/49% DCM/1% water and the supernatant analyzed via HPLC and LCMS to monitor the conversion of 4-(3-amino-5-ethoxycarbonylphenyl)-benzamide to 4-(3-phenylcarbonylamino-5-ethoxycarbonylphenyl)benzamide.

Step 6B

Alternatively, 4-(3-phenylcarbonylamino-5-ethoxycarbonylphenyl)benzamide was prepared as follows: To 4-(3-amino-5-ethoxycarbonylphenyl)benzamide (0.52 mmol) was added DCM (5 ml) and DMA (0.73 ml, 4.16 mmol) followed by the benzoyl chloride (2.08 mmol) and the reaction mixture was stirred at room temperature overnight. The solvents were drained and the resin washed with DCM (4×). A small amount of resin was cleaved with a solution of 50% TFA/49% DCM/1% water and the supernatant analyzed via HPLC and LCMS LCMS to monitor the conversion of of 4-(3-amino-5-ethoxycarbonylphenyl)benzamide to 4-(3-phenylcarbonylamino-5-ethoxycarbonylphenyl)benzamide.

Step 7

To the resin bound 4-(3-phenylcarbonylamino-5-ethoxycarbonylphenyl)benzamide (0.52 mmol) was added THF (5 ml) and potassium trimethylsilanoate (0.27 g, 2.08 mmol) and the reaction mixture was agitated at room temperature for 4 h. The solvents were drained and the resin washed with THF (4×), MeOH (4×) and finally DCM (2×). A small amount of resin was cleaved with a solution of 50% TFA/49% DCM/1% water and the supernatant analyzed via HPLC and LCMS to monitor the conversion of 4-(3-phenylcarbonylamino-5-ethoxycarbonylphenyl)-benzamide to 4-(3-phenylcarbonylamino-5-carboxyphenyl)benzamide.

Step 8

To the resin bound 4-(3-phenylcarbonylamino-5-carboxyphenyl)benzamide (0.52 mmol) was added DMF (5 ml), followed by dimethylamine hydrochloride (170 mg, 2.08 mmol), HATU (0.79 g, 2.08 mmol), and DIEA (0.73 ml, 4.16 mmol) and the mixture agitated at room temperature overnight. The solvents were drained and the resin washed with DMF (4×) followed by DCM (2×). A small amount of resin was cleaved with a solution of 50% TFA/49% DCM/1% water and the supernatant analyzed via HPLC and LCMS to monitor the conversion of 4-(3-phenylcarbonyl-amino-5-carboxyphenyl)benzamide to 4-(5-N,N-dimethylaminocarbonyl-3-phenylcarbonylamino-phenyl)benzamide.

Step 9

To the resin (0.52 mmol) in Step 8 above, was added 5 ml of a solution of 50% TFA/49% DCM/1% water and the mixture agitated for 30 minutes. The solvents were drained into a round bottom flask and concentrated to afford the product as a tan colored solid. The solid was dissolved in hot ethyl acetate and slowly added to hexane to afford precipitation of N-hydroxy-4-[5-(N,N-dimethylaminocarbonyl)-3-(phenylcarbonylamino)-phenyl]benzamide as a white solid (80 mg, 30% yield). ¹H NMR(DMSO-d6): 11.28 (s, 1H), 10.45 (s, 1H), 8.18 (br t, 1H), 7.99-7.44 (m, 11H), 3.00 (br d, 6H); MS: calc 403.4; found 402.0 (M−1), 404.2 (M+1).

Proceeding a described in Example 13 above, but using suitable starting materials the following compounds of Formula I were prepared:

N-hydroxy-4-[5-(N-benzylaminocarbonyl)-3-(phenylcarbonylamino)-phenyl]benzamide: ¹H NMR(DMSO-d6): 11.29 (s, 1H), 10.49 (s, 1H), 9.19 (m, 1H), 8.34 (s, 2H), 8.10-7.79 (m, 7H), 7.59-7.43 (m, 3H), 7.35-7.33 (m, 5H) 4.52 (d, 2H, J=5.9 Hz); MS: calc 465.5; found 464.4 (M−1), 466.2 (M+1).

N-hydroxy-4-[5-(carboxy)-3-(phenylcarbonylamino)-phenyl]benzamide: ¹H NMR(DMSO-d6): 11.29 (s, 1H), 10.53 (s, 1H), 8.49 (s, 1H), 8.42 (s, 1H), 8.02-7.53 (m, 10H); MS: calc 376.37; found 374.8 (M−1), 377.2 (M+1).

N-hydroxy-4-[5-(morpholin-1-ylcarbonyl)-3-(phenylcarbonylamino)-phenyl]benzamide: ¹H NMR(DMSO-d6): 11.30 (s, 1H), 10.52 (s, 1H), 10.46 (s, 1H), 8.46 (m, 1H), 8.39 (m, 1H), 8.20 (m, 1H), 7.99-7.44 (m, 12H), 3.62 (br s 2, 8H); MS: calc 445.5; found 444.4 (M−1), 444.6 (M+1).

N-hydroxy-4-[5-(N,N-dimethylaminocarbonyl)-3-(4-methylphenylcarbonylamino)-phenyl]benzamide: ¹H NMR(DMSO-d6): 11.28 (s, 1H), 10.35 (s, 1H), 8.17 (m, 1H), 7.91-7.73 (m, 8H), 7.42-7.33 (m, 2H), 2.99 (br d 2, 6H), 2.38 (2, 3H); MS: calc 417.4; found 416.4 (M−1), 418.6 (M+1).

N-hydroxy-4-[5-(morpholin-1-ylcarbonyl)-3-(4-methylphenylcarbonylamino)-phenyl]benzamide: ¹H NMR(DMSO-d6): 10.37 (s, 1H), 8.20 (s, 1H), 7.91-7.73 (m, 8H), 7.44-7.34 (m, 2H), 3.62 (br 2, 8H), 2.38 (s, 3H); MS: calc 459.5; found 458.4 (M−1), 460.6 (M+1).

N-hydroxy-4-[5-(N-phenylaminocarbonyl)-3-(4-methylphenylcarbonylamino)-phenyl]benzamide: ¹H NMR(DMSO-d6): 11.26 (s, 1H), 10.40 (s, 1H), 10.32 (s, 1H), 8.32 (d, 2H, J=8.4 Hz), 7.95-7.71 (m, 1H), 7.34-7.29 (m, 4H), 7.06 (t, 1H, J=7.8 Hz), 2.34 (s, 3H); MS: calc 465.5; found 464.4 (M−1), 466.2 (M+1).

N-hydroxy-4-[5-(N,N-dimethylaminocarbonyl)-3-(4-methoxyphenylcarbonylamino)-phenyl]benzamide: ¹H NMR(DMSO-d6): 11.26 (s, 1H), 10.40 (s, 1H), 8.00-7.76 (m, 9H), 7.08 (d, 2H, J=8.6 Hz), 8.20 (m, 1H), 7.99-7.44 (m, 12H), 3.62 (br 2, 8H); MS: calc 433.5; found 432.6 (M−1), 434.6 (M+1).

N-hydroxy-4-[5-(morpholin-1-ylcarbonyl)-3-(4-methoxyphenylcarbonylamino)-phenyl]benzamide: ¹H NMR(DMSO-d6): 11.29 (s, 1H), 10.36 (s, 1H), 10.30 (s, 1H), 8.45-8.40 (m, 1H), 8.19-7.73 (m, 9H), 7.42 (s, 1H), 7.08 (d, 2H, J=8.6 Hz), 3.83 (s, 3H), 3.61 br m, 8H); MS: calc 475.5; found 474.2 (M−1), 476.5 (M+1).

N-hydroxy-4-[5-(N-phenylaminocarbonyl)-3-(4-methoxyphenylcarbonylamino)-phenyl]benzamide: ¹H NMR(DMSO-d6): 11.30 (s, 1H), 10.36 (s, 1H), 8.39-8.34 (m, 2H), 8.04-7.76 (m, 8H), 7.39-7.33 (m, 2H), 7.15-7.06 (m, 4H), 3.84 (s, 3H); MS: calc 481.5; found 480.2 (M−1), 482.2 (M+1).

N-hydroxy-4-[5-(N,N-dimethylaminocarbonyl)-3-(3,4-dimethoxyphenylcarbonylamino)-phenyl]benzamide: ¹H NMR(DMSO-d₆): 11.30 (s, 1H), 10.27 (s, 1H), 8.19 (s, 1H), 7.89-7.43 (m, 9H), 7.10 (m, 1H), 3.84 (br d, 6H), 3.00 (br d, 6H); MS: calc 463.49; found 462.4 (M−1), 464.4 (M+1).

N-hydroxy-4-[5-(morpholin-1-ylcarbonyl)-3-(3,4-dimethoxyphenylcarbonylamino)-phenyl]benzamide: ¹H NMR(DMSO-d6): 11.30 (s, 1H), 10.35 (m, 2H), 8.43 (d, 1H, J+1.5 Hz), 8.21 (m, 1H), 7.94-7.44 (m, 8H), 7.11 (d, 1H, 8.6 Hz), 3.84 (br d, 6H), 3.63 (br m, 8H); MS: calc 505.5; found 504.2 (M−1), 506.4 (M+1).

N-hydroxy-4-[5-(N-phenylaminocarbonyl)-3-(3,4-dimethoxyphenylcarbonylamino)-phenyl]benzamide: ¹H NMR(DMSO-d6): 11.31 (s, 1H), 10.37 (s, 1H), 8.41 (s, 1H), 8.32 (s, 1H), 8.05-7.08 (m, 13H), 3.84 (d, 6H, J=3.5 Hz); MS: calc 511.5; found 510.4 (M−1), 512.6 (M+1).

N-hydroxy-4-[5-(piperidin-1-ylcarbonyl)-3-(4-methylphenylcarbonylamino)-phenyl]benzamide: ¹H NMR(DMSO-d6): 11.28 (s, 1H), 10.36 (s, 1H), 9.06 (s, 1H), 8.18 (s, 1H), 7.91-7.72 (m, 8H), 7.38-7.33 (m, 3H), 3.31 (m, 4H), 2.38 (s, 3H), 1.61 (m, 6H); MS: calc 457.53; found 456.0 (M−1), 458.4 (M+1).

N-hydroxy-4-[5-(piperazin-1-ylcarbonyl)-3-(4-methylphenylcarbonylamino)-phenyl]benzamide: ¹H NMR(DMSO-d6): 11.33 (s, 1H), 10.41 (s, 1H), 9.08 (s, 1H), 8.17 (s, 1H), 7.99-7.73 (m, 8H), 7.51 (s, 1H), 7.35 (d, 2H, 8.4 Hz), 3.31 (s, 4H), 3.18 (br s, 4H), 2.39 (s, 3H); MS: calc 458.5; found 457.4 (M−1), 459.4 (M+i).

N-hydroxy-4-[5-(N-methylaminocarbonyl)-3-(4-methylphenylcarbonylamino)-phenyl]benzamide: ¹H NMR(DMSO-d6): 11.30 (s, 1H), 10.43 (s, 1H), 9.07 (s, 1H), 8.46 (s, 1H), 8.40 (s, 1H), 7.94-7.74 (m, 7H), 7.36 (d, 2H, 8.1 Hz), 2.50 (s, 3H), 2.39 (s, 3H); MS: calc 403.4; found 402.2 (M−1), 404.4 (M+1).

N-hydroxy-4-[5-(N-carboxymethyl-N-methylaminocarbonyl)-3-(4-methylphenylcarbonylamino)-phenyl]benzamide: ¹H NMR(DMSO-d6): 11.30 (s, 1H), 10.33 (br m, 1H), 9.01 (s, 1H), 8.19 (s, 1H), 8.11 (s, 1H), 7.87-7.80 (m, 5H), 7.71-7.63 (m, 3H), 7.35-7.28 (m, 3H), 4.11 (s, 2H), 2.96 (s, 3H), 2.33 (s, 3H); MS: calc 461.5; found 460.4 (M−1), 462.4 (M+1).

N-hydroxy-4-[5-(N-aminocarbonylmethylaminocarbonyl)-3-(4-methylphenylcarbonyl-amino)-phenyl]benzamide: ¹H NMR(DMSO-d6): 11.30 (s, 1H), 10.41 (s, 1H), 9.08 (s, 1H), 8.35 (s, 1H), 8.30 (s, 1H), 7.97-7.80 (m, 8H), 7.40-7.33 (m, 3H), 4.09 (br s, 2H), 2.38 (s, 3H); MS: calc 446.5; found 445.4 (M−1), 447.4 (M+1).

N-hydroxy-4-(5-[(N-(2-N-methylaminoethyl)-N-(methyl)aminocarbonyl)-3-(4-methylphenylcarbonylamino)]-phenyl}benzamide: ¹H NMR(DMSO-d6): 11.31 (s, 1H), 10.42 (s, 1H), 9.08 (s, 1H), 8.61 (br s, 1H), 8.15-7.75 (m, 9H), 7.38-7.35 (m, 2H), 3.39 (s, 2H), 3.02 (s, 3H), 2.63 (s, 2H), 2.48 (s, 3H), 2.40 (s, 3H); MS: calc 460.5; found 459.2 (M−1), 461.6 (M+1).

N-hydroxy-4-[5-(N-carboxymethylaminocarbonyl)-3-(4-methylphenylcarbonyl-amino)-phenyl]benzamide: ¹H NMR(DMSO-d6): 9.09 (s, 1H), 8.50-8.40 (m, 2H), 7.94-7.77 (m, 7H), 7.36 (d, 2H, 7.9 Hz), 3.36 (s, 2H), 2.39 (s, 3H); MS: calc 447.4; found 446.6 (M-1), 448.4 (M+1).

N-hydroxy-4-[5-(N,N-dimethylaminocarbonyl)-3-(3,4-methylenephenylcarbonyl-amino)-phenyl]benzamide: ¹H NMR(DMSO-d6): 10.23 (s, 1H), 8.12 (s, 1H), 7.86-7.37 (m, 7H), 7.02 (d, 2H, 8.2 Hz), 6.08 (s, 2H), 2.93 (br d, 6H); MS: calc 447.4; found 446.6 (M−1), 448.2 (M+1).

N-hydroxy-4-[5-(morpholin-4-ylcarbonyl)-3-(3,4-methylenephenylcarbonylamino)-phenyl]benzamide: ¹H NMR(DMSO-d6): 11.30 (s, 1H), 10.35 (s, 1H), 10.29 (s, 1H), 8.45-8.39 (m, 2H), 8.19 (s, 1H), 7.89-7.43(m, 5H), 7.51 (s, 1H), 7.07 (d, 2H, 8.2 Hz), 6.13 (s, 2H), 3.62 (br s, 8H; MS: calc 489.5; found 488.6 (M−1), 490.4 (M+1).

N-hydroxy-4-[5-(N-phenylaminocarbonyl)-3-(3,4-methylenephenylcarbonylamino)-phenyl]benzamide: ¹H NMR(DMSO-d6): 11.26 (s, 1H), 10.31 (s, 1H), 9.02 (br s, 1H), 8.32-8.29 (m, 2H), 7.95-7.01 (m, 13H), 6.09 (s, 2H); MS: calc 495.5; found 494.4 (M−1), 496.4 (M+1).

N-hydroxy-4-[5-(N,N-dimethylaminocarbonyl)-3-(2,4-dichlorophenylcarbonyl-amino)-phenyl]benzamide: ¹H NMR(DMSO-d6): 11.29 (s, 1H), 10.78 (s, 1H), 8.05 (s, 1H), 7.85-7.46 (m, 9H), 2.98 (br d, 6H); MS: calc 472.3; found 471.2 (M−1), 473.3 (M+1).

N-hydroxy-4-[3-(4-chlorophenylcarbonylamino)-5-(morpholin-4-ylcarbonyl)-phenyl]benzamide: ¹H NMR(DMSO-d6): 11.23 (s, 1H), 10.74 (s, 1H), 9.02 (br s, 1H), 8.02 (s, 1H), 7.83-7.42 (m, 9H), 3.57 (br s, 8H); MS: calc 514.4; found 513.4 (M−1), 515.4 (M+1).

N-hydroxy-4-[5-(N-phenylaminocarbonyl)-3-(2,4-dichlorophenylcarbonylamino)-phenyl]benzamide: ¹H NMR(DMSO-d6): 11.25 (s, 1H), 10.80 (s, 1H), 10.34 (s, 1H), 9.02 (s, 1H), 8.21 (s, 1H), 8.17 (s, 1H), 7.98-7.28 (m, 13H), 7.09-7.03 (m, 1H); MS: calc 520.4; found 519.4 (M−1), 521.6 (M+1).

N-hydroxy-4-[5-(N,N-dimethylaminocarbonyl)-3-(4-chlorophenylcarbonylamino)-phenyl]benzamide: ¹H NMR(DMSO-d6): 11.29 (s, 1H), 10.53 (s, 1H), 8.17-7.45 (m, 11H), 2.99 (br d, 6H); MS: calc 437.9; found 436.2 (M−1), 438.2 (M+i).

N-hydroxy-4-[5-(morpholin-4-ylcarbonyl)-3-(4-chlorophenylcarbonylamino)-phenyl]benzamide: ¹H NMR(DMSO-d6): 11.29 (s, 1H), 10.53 (s, 1H), 8.18-7.46 (m, 11H), 3.62 (br s, 8H); MS: calc 479.9; found 478.0 (M−1), 480.2 (M+1).

N-hydroxy-4-[5-(N-ethyl-N-methylaminocarbonyl)-3-(4-methylphenylcarbonylamino)-phenyl]benzamide: ¹H NMR(DMSO-d6): 11.29 (s, 1H), 10.36 (s, 1H), 8.19 (s, 1H), 7.94-7.74 (m, 7H), 7.40-7.34 (m, 3H), 3.47 (m, 2H), 2.96 (s, 3H), 2.39 (s, 3H), 1.25 (m, 3H); MS: calc 431.5; found 430.4 (M−1), 432.2 (M+1).

N-hydroxy-4-[5-(4-methylpiperazin-1-ylcarbonyl)-3-(4-methylphenylcarbonyl-amino)-phenyl]benzamide: ¹H NMR(DMSO-d6): 11.31 (s, 1H), 10.98 (s, 1H), 10.47 (s, 1H), 8.20-7.33 (m, 11H), 3.11 (br s, 4H), 2.77 (s, 4H), 2.46 (s, 3H), 2.38 (s, 3H); MS: calc 472.5; found 471.4 (M−1), 473.6 (M+1).

N-hydroxy-4-[5-(3-(RS)-aminocarbonylpiperidin-1-ylcarbonyl)-3-(4-methylphenyl-carbonylamino)-phenyl]benzamide: ¹H NMR(DMSO-d6): 11.29 (s, 1H), 10.38 (s, 1H), 9.07 (s, 1H), 8.17 (s, 1H), 7.94-7.73 (m, 8H), 7.40-7.34 (m, 3H), 3.90 (s, 2H), 3.26 (m, 2H), 2.89 (m, 1H), 2.39 (s, 3H), 1.63 (m, 2H), 1.24 (m, 2H); MS: calc 500.6; found 499.6 (M−1), 501.6 (M+1).

N-hydroxy-4-[5-(N,N-diethylaminocarbonyl)-3-(4-methylphenylcarbonylamino)-phenyl]benzamide: ¹H NMR(DMSO-d6): 11.28 (s, 1H), 10.36 (s, 1H), 8.17 (s, 1H), 7.91-7.73 (m, 7H), 7.36-7.33 (m, 1H), 2.48 (m, 4H), 2.38 (m, 3H), 1.12 br s, 6H); MS: calc 445.5; found 444.4 (M−1), 446.6 (M+1).

N-hydroxy-4-[5-(N-(2-N-methylaminoethyl)aminocarbonyl)-3-(4-methylphenyl-carbonyl-amino)-phenyl]benzamide: ¹H NMR(DMSO-d6): 11.31 (s, 1H), 10.42 (s, 1H), 8.86 (m, 1H), 8.58 (m, 2H), 8.37 (s, 1H), 8.30 (s, 1H), 7.97-7.79 (m, 8H), 7.34 (d, 2H, J=8.1 Hz), 3.40 (m, 2H), 3.10 (m, 2H), 2.65 (m, 3H), 2.35 (s, 3H); MS: calc 446.5; found 445.6 (M−1), 447.6 (M+1).

N-hydroxy-4-[5-(4-hydroxypiperidin-1-ylcarbonyl)-3-(4-methylphenylcarbonyl-amino)-phenyl]benzamide: ¹H NMR(DMSO-d6): 11.23 (s, 1H), 10.31 (s, 1H), 8.14 (m, 1H), 7.86-7.68 (m, 6H), 7.34-7.28 (m, 4H), 3.68 (m, 4H), 2.33 (s, 3H), 1.32 (br m, 4H); MS: calc 473.5; found 472.6 (M−1), 474.4 (M+1).

N-hydroxy-4-[5-(pyrrolidin-1-ylcarbonyl)-3-(4-methylphenylcarbonylamino)-phenyl]benzamide: ¹H NMR(DMSO-d6): 10.37 (s, 1H), 8.20 (s, 1H), 8.01-7.74 (m, 8H), 7.53-7.33 (m, 4H), 3.48 (m, 4H), 2.38 (s, 3H), 1.85 (br m, 4H); MS: calc 443.5; found 442.4 (M−1), 444.4 (M+1).

N-hydroxy-4-[5-(3-(RS)-hydroxymethylpiperidin-1-ylcarbonyl)-3-(4-methylphenyl-carbonylamino)-phenyl]benzamide ¹H NMR(DMSO-d6): 10.37 (s, 1H), 8.19 (m, 1H), 7.92-7.73 (m, 8H), 7.38-7.33 (m, 3H), 4.50-4.35 (m, 1H), 3.50-3.10 (m, 5H), 2.38 (s, 3H), 1.73-1.13 (m, 5H); MS: calc 487.6; found 486.4 (M−1), 488.4 (M+1).

N-hydroxy-4-[5-(2-(RS)-hydroxymethylpiperidin-1-ylcarbonyl)-3-(4-methylphenyl-carbonylamino)-phenyl]benzamide ¹H NMR(DMSO-d6): 11.36 (s, 1H), 10.56 (s, 1H), 9.17 (s, 2H), 8.65 (s, 1H), 8.51 (s, 1H), 8.13-7.81 (m, 7H), 7.36 (d, 2H, J=8.4 Hz), 4.51 (m, 2H), 3.39-2.91 (m, 4H), 2.39 (s, 3H), 1.88-1.54 (m, 6H); MS: calc 487.5; found 486.4 (M−1), 488.8 (M+1).

N-hydroxy-4-[5-(N-2-hydroxyethyl-N-methylaminocarbonyl)-3-(4-methylphenyl-carbonylanlino)-phenyl]benzamide: ¹H NMR(DMSO-d6): 11.28 (s, 1H), 10.36 (s, 1H), 8.17-7.72 (m, 8H), 7.44-7.33 (m, 3H), 3.83 (m, 2H), 3.37 (m, 2H), 3.00 (s, 3H), 2.38 (s, 3H); MS: calc 447.5; found 446.6 (M−1), 448.2 (M+1).

N-hydroxy-4-[5-(N-4-chlorobenzyl]aminocarbonyl)-3-(4-methylphenyl-carbonylamino)-phenyl]benzamide: ¹H NMR(DMSO-d6): 10.39 (s, 1H), 8.32 (m, 2H), 7.95-7.78 (m, 7H), 7.38-7.33 (m, 6H), 4.50-4.40 (m, 2H), 2.38 (s, 3H); MS: calc 513.9; found 512.4 (M−1), 514.4 (M+1).

N-hydroxy-4-[5-(N-3-methylbutylaminocarbonyl)-3-(4-methylphenylcarbonyl-amino)-phenyl]benzamide: MS: calc 459.5; found 458.4 (M−1), 460.4 (M+1).

N-hydroxy-4-[5-(N-2-phenylethylaminocarbonyl)-3-(4-methylphenylcarbonylamino)-phenyl]benzamide: ¹H NMR(DMSO-d6): 11.24 (s, 1H), 10.34 (s, 1H), 8.67-8.63 (m, 1H), 8.26-8.22 (m, 2H), 7.89-7.72 (m, 7H), 7.32-7.14 (m, 7H), 3.50-3.42 (m, 2H), 2.84-2.79 (m, 2H), 2.33 (s, 3H); MS: calc 493.6; found 492.6 (M−1), 494.4 (M+1).

N-hydroxy-4-{5-[N-2-(4-methoxyphenyl)ethylaminocarbonyl)]-3-(4-methylphenyl-carbonylamino)-phenyl}benzamide: MS: calc 523.6; found 522.6 (M−1), 524.2 (M+1).

N-hydroxy-4-[5-(N-methyl-N-methoxyaminocarbonyl)-3-(4-methylphenylcarbonyl-amino)phenyl]benzamide: MS: calc 433.5; found 432.4 (M−1), 434.4 (M+1).

N-hydroxy-4-[5-(N-methyl-N-phenylaminocarbonyl)-3-(4-methylphenyl-carbonylamino)-phenyl]benzamide: MS: calc 479.5; found 478.0 (M−1), 480.2 (M+1).

N-hydroxy-4-[5-(N-benzylaminocarbonyl)-3-(4-methylphenylcarbonylamino)-phenyl]benzamide: MS: calc 479.5; found 478.0 (M−1), 480.4 (M+1).

N-hydroxy-4-[5-(N-2-hydroxyethylaminocarbonyl)-3-(4-methylphenyl-carbonylamino)-phenyl]benzamide: MS: calc 433.4; found 432.4 (M−1), 434.6 (M+1).

N-hydroxy-4-{5-[N,N-bis(2-hydroxyethyl)aminocarbonyl]-3-(4-methylphenyl-carbonylamino)-phenyl]benzamide: MS: calc 477.5; found 476.2 (M−1), 479.4 (M+1).

N-hydroxy-4-[5-(4-(RS)-hydroxymethylpiperidin-1-ylcarbonyl)-3-(4-methylphenyl-carbonylamino)-phenyl]benzamide: MS: calc 487.5; found 486.4 (M−1), 488.6 (M+1).

N-hydroxy-4-[5-(3-(RS)-methoxycarbonylpiperidin-1-ylcarbonyl)-3-(4-methylphenyl-carbonylamino)-phenyl]benzamide: MS: calc 529.6; found 528.6 (M−1), 530.4 (M+1).

N-hydroxy-4-[5-(4-(RS)-aminocarbonylpiperidin-1-ylcarbonyl)-3-(4-methylphenyl-carbonylamino)-phenyl]benzamide: MS: calc 500.6; found 501.6 (M+1).

N-hydroxy-4-[5-(2-(S)-aminocarbonylpyrrolidin-1-ylcarbonyl)-3-(4-methylphenyl-carbonylamino)-phenyl]benzamide: MS: calc 486.5; found 485.4 (M−1), 487.6 (M+1).

N-hydroxy-4-[5-(4-(RS)-methoxycarbonylpiperidin-1-ylcarbonyl)-3-(4-methyl-phenylcarbonylamino)-phenyl]benzamide: MS: calc 515.6; found 514.4 (M−1), 516.6 (M+1).

N-hydroxy-4-{5-[(N-(N-methylaminocarbonyl)-N-(methyl)aminocarbonyl]-3-(4-methylphenylcarbonylamino)-phenyl]benzamide: MS: calc 460.5; found 459.2 (M−1), 461.2 (M+1).

N-hydroxy-4-[5-(phenylacetylamino)-3-(pyrrolidine-1-carbonyl)phenyl]benzamide. ¹H NMR (DMSO-d₆): 10.40 (1H, s), 9.06 (1H, br s), 7.95 (1H, s), 7.83 (2H, d, J=7.6 Hz), 7.78 (1H, s), 7.69 (2H, d, J=7.6 Hz), 7.46 (1H, s), 7.33-7.23 (5H, m), 3.66 (1H, s), 3.46 (2H, t, J=7.5 Hz), 3.40 (2H, t, J=7.5 Hz), 1.88-1.78 (4H, m). MS: 444.4 (M+1); 442.3 (M−1).

N-hydroxy-4-[5-(3-phenyl-propionylamino)-3-(pyrrolidine-1-carbonyl)phenyl]-benzamide. ¹H NMR (DMSO-d₆): 10.06 (1H, s), 7.86 (1H, d, J=1.5 Hz), 7.77 (2H, d, J=8.3 Hz), 7.70 (1H, d, J=1.5 Hz), 7.63 (2H, d, J=8.3 Hz), 7.39 (1H, d, J=1.5 Hz), 7.22-7.10 (5H, m), 3.40 (2H, t, J=6.7 Hz), 3.34 (2H, t, J=6.7 Hz), 2.86 (2H, t, J=7.6 Hz), 2.59 (2H, t, J=7.6 Hz), 1.83-1.74 (4H, m). MS: 458.3 (M+1); 456.1 (M−1).

N-hydroxy-4-[5-(3-phenylacryloylamino)-3-(pyrrolidine-1-carbonyl)phenyl]-benzamide. ¹H NMR (DMSO-d₆): 11.28 (1H, s), 10.45 (1H, s), 9.06 (1H, s), 8.04 (1H, s), 7.90 (1H, s), 7.85 (2H, d, J=7.9 Hz), 7.73 (2H, d, J=7.9 Hz), 7.64-7.59 (3H, m), 7.50 (1H, s), 7.46-7.40 (3H, m), 6.82 (1H, d, J=15.6 Hz), 3.50-3.44 (4H, m), 1.91-1.82 (4H, m). MS: 456.1 (M+1); 454.2 (M−1).

N-hydroxy-4-[3-(4-methoxy-2-methylbenzoylamino)-5-(pyrrolidine-1-carbonyl)-phenyl]-benzamide. ¹H NMR (DMSO-d₆): 10.35 (1H, s), 8.13 (1H, s), 7.92 (1H, s), 7.85 (2H, d, J=7.8 Hz), 7.72 (2H, d, J=7.8 Hz), 7.49 (2H, m), 6.86 (2H, m), 3.79 (3H, s), 3.46 (4H, m), 2.41 (3H, s), 1.87 (4H, m); MS: 472.4 (M−1), 474.4 (M+1).

N-hydroxy-4-[3-(4-methoxy-2-methyl-benzoylamino)-5-(piperidine-1 arbonyl)-phenyl]benzamide. ¹H NMR (DMSO-d₆): 10.35 (1H, s), 8.11 (1H, s), 7.86 (2H, d, J=8.2 Hz), 7.78 (1H, s), 7.71 (2H, d, J=8.2 Hz), 7.49 (1H, d, J=8.0 Hz), 7.35 (1H, s), 6.85 (3H, m), 3.79 (3H, s), 3.59 (2H, m), 3.34 (2H, m), 2.41 (3H, s), 1.55 (6H, m); MS: 486.4 (M−1), 488.5 (M+1).

N-hydroxy-4-[3-(2,4-dimethylbenzoylamino)-5-(pyrrolidine-1-carbonyl)phenyl]-benzamide. ¹H NMR (DMSO-d₆): 10.41 (1H, s), 8.12 (1H, s), 7.92 (1H, s), 7.85 (2H, d, J=7.9 Hz), 7.72 (2H, d, J=7.9 Hz), 7.50 (1H, s), 7.40 (1H, d, J=7.6 Hz), 7.11 (3H, m), 3.47 (4H, m), 2.38 (3H, s), 2.33 (3H, s), 1.87 (4H, m); MS: 456.3 (M−1), 458.1 (M+1).

N-hydroxy-4-[3-(2,4-dimethylbenzoylamino)-5-(piperidine-1-carbonyl)phenyl]-benzamide. ¹H NMR (DMSO-d₆): 10.41 (1H, s), 8.12 (1H, s), 7.85 (2H, d, J=7.7 Hz), 7.79 (1H, s), 7.71 (2H, d, J=7.7 Hz), 7.4 (1H, d, J=7.7 Hz), 7.36 (1H, s), 7.11 (3H, m), 3.59 (2H, m), 3.34 (2H, m), 2.38 (3H, s), 2.33 (3H, s), 1.57 (6H, m); MS: 470.3 (M−1), 472.4 (M+1).

N-hydroxy-4-[5-(2,4-dimethylbenzoylamino)-3-(diethylaminocarbonyl)phenyl]-benzamide. MR (DMSO-d₆): 10.41 (1H, s), 8.10 (1H, s), 7.85 (2H, d, J=7.3 Hz), 7.78 (1H, s), 7.71 (2H, d, J=7.3 Hz), 7.40 (1H, d, J=6.4 Hz), 7.34 (1H, s), 7.11 (3H, m), 3.45 (2H, m), 3.25 (2H, m), 2.39 (3H, s), 2.33 (3H, s), 1.15(6H, m); MS: 458.1 (M−1), 460.4 (M+1).

N-hydroxy-4-[5-(4-methoxy-2-methylbenzoylamino)-3-(diethylaminocarbonyl)-phenyl]-benzamide. ¹H NMR (DMSO-d₆): 10.35 (1H, s), 8.09 (1H, s), 7.85 (2H, d, J=7.7 Hz), 7.78 (1H, s), 7.71 (2H, d, J=7.7 Hz), 7.49 (1H, d, J=7.7 Hz), 7.33 (1H, s), 6.85 (3H, m), 3.79 (3H, s), 3.44 (2H, m), 3.28 (2H, m), 2.41 (3H, s), 1.13 (6H, m); MS: 474.4 (M−1), 476.2 (M+1).

N-hydroxy-4-[3-(dimethylaminocarbonyl)-5-(indol-3-ylmethylcarbonylamino)phenyl]-benzamide: ¹HNMR(DMSO-d6): 11.28 (s, 1H), 10.35 (s, 1H), 8.17 (m, 1H), 7.91-7.73 (m, 8H), 7.42-7.33 (m, 2H), 2.99 (br d 2, 6H), 2.38 (2, 3H); MS: calc 417.4; found 416.4 (M−1), 418.6 (M+1).

N-hydroxy-4-[3-(dimethylaminocarbonyl)-5-(pyridin-4-ylmethylcarbonylamino)-phenyl]-benzamide: ¹H NMR (DMSO-d6): 11.26 (s, 1H), 10.40 (s, 1H), 10.32 (s, 1H), 8.32 (d, 2H, J=8.4 Hz), 7.95-7.71 (m, 11H), 7.34-7.29 (m, 4H), 7.06 (t, 1H, J=7.8 Hz), 2.34 (s, 3H); MS: calc 465.5; found 464.4 (M−1), 466.2 (M+1).

N-hydroxy-4-{5-(dichlorophenylcarbonylamino)-3-[N-methyl-N-(2-hydroxyethyl)-aminocarbonyl]phenyl}-benzamide: ¹H NMR (DMSO-d6): 11.28 (s, 1H), 10.38 (s, 1H), 10.30 (s, 1H), 8.32 (s, 1H), 7.93-7.65 (m, 7H), 7.52-7.25 (m, 2H), 3.5 (m, 2H), 3.2 (m, 2H), 3.00 (s, 3H); MS: calc 501.09; found 500.0 (M−1), 502.2 (M+1).

### Example 14

### Synthesis of N-hydroxy-4-[3-(benzoylaminomethyl)phenyl]benzamide

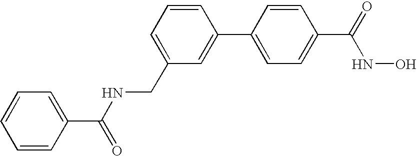 Step 1

To a prestirred (30 min) solution of (3-bromobenzyl)-carbamic acid benzyl ester (2.2 g, 6.8 mmol) and tetrakis(triphenylphosphine)palladium(0) (0.24 g, 0.2 mmol) in toluene (50 ml) and methanol (10 ml) was added (4-methoxycarbonylphenyl)boronic acid (1.3 g, 7.2 mmol) and K₂CO₃ (4.7 g, 34 mmol). The resulting slurry was heated to 75° C. for 3 h, then cooled to room temperature and diluted with ethyl acetate (200 ml) and washed with water (300 ml). The organic layer was dried (MgSO₄), filtered and concentrated to a brown oil. Flash chromatography (25% ethyl acetate/hexanes) provided 1.9 g (74%) of 3′-(benzyloxycarbonylaminomethyl)-biphenyl-4-carboxylic acid methyl ester as a white solid.

Step 2

To a solution of 3′-(benzyloxycarbonylamino-methyl)-biphenyl-4-carboxylic acid methyl ester (0.5 g, 1.3 mmol) in methanol (10 ml) and THF (3 ml) was added NaOH (0.6 g dissolved in 5 ml of H₂O). The solution was stirred 2 h at room temperature, then acidified to pH=2 with 1N HCl, dilited with H₂O (150 ml) and extracted into ethyl acetate (150 ml). The organic layer was dried (MgSO₄), filtered and concentrated to collect 0.47 g (98%) of 3′-(benzyloxycarbonylamino-methyl)-biphenyl-4-carboxylic acid as a white solid and this material was taken forward without further purification.

Step 3

To a solution of 3′-(benzyloxycarbonylamino-methyl)-biphenyl-4-carboxylic acid (0.47 g, 1.3 mmol), O-tert-butylhydroxylamine hydrochloride (0.2 g, 1.6 mmol) and TEA (0.9 ml) in DMF (10 ml) was added BOP reagent (0.63 g, 1.4 mmol). After stirring 3 h at room temperature, the solution was diluted with ethyl acetate (100 ml) and washed with 1N HCl (2×100 ml), then mild NaHCO₃ (2×100 ml). The organic layer was dried (MgSO₄), filtered and concentrated. Flash chromatography (40% ethyl acetate/hexane) provided 0.47 g (82%) of (4′-tert-butoxycarbamoyl-biphenyl-3-ylmethyl)-carbamic acid benzyl ester as a white solid.

Step 4

A mixture of (4′-tert-butoxycarbamoyl-biphenyl-3-ylmethyl)-carbamic acid benzyl ester (0.47 g, 1.1 mmol) and palladium (10% on charcoal, 50 mg) in methanol (15 mL) was stirred under a hydrogen atmosphere for 35 min and then the suspension was filtered through a plug of celite to remove the palladium. Concentration of the solvent resulted in the isolation of 0.33 g (100%) of crude 3′-aminomethyl-biphenyl-4-carboxylic acid tert-butoxy-amide.

Step 5

To a solution of 3′-aminomethyl-biphenyl-4-carboxylic acid tert-butoxy-amide (0.10 g, 0.34 mmol), benzoic acid (50 mg, 0.4 mmol) and TEA (0.4 mL) in DMF (2 mL) was added BOP reagent (0.16 g, 0.37 mmol). The solution was stirred 45 minutes at room temperature, then diluted with ethyl acetate (75 ml) and washed with 1N HCl (2×100 ml), then mild NaHCO₃ (2×100 ml). The organic layer was dried (MgSO₄), filtered and concentrated to collect a white foam. To the foam was added 30% HBr/acetic acid (2 mL). After 30 minutes of stirring at room temperature, the reaction solution was concentrated under vacuum, and the resulting solid was subjected to reverse phase HPLC to provide 98 mg (83%) of the title compound.

¹H NMR (DMSO-d₆): 11.17 (1H, s), 9.00 (1H, t, J=5.8 Hz), 7.81 (2H, d, J=7.7 Hz), 7.76 (2H, d, J=7.7 Hz), 7.64 (2H, d, J=7.4 Hz), 7.59 (1H, s), 7.52-7.26 (6H, m), 4.48 (2H, d, J=5.8 Hz). MS: 347.2 (M+1); 345.2 (M−1).

Proceeding as described in Example 14 above, but using suitable starting materials the following compounds were prepared.

N-hydroxy-4-[3-(2-pyridin-3-yl-acetylaminomethylphenyl)]benzamide. ¹H NMR (DMSO-d₆): 11.20 (1H, s), 8.98 (1H, br s), 8.64 (1H, d, J=5.6 Hz), 8.44 (1H, s), 8.40 (1H, d, J=3.5 Hz), 7.76 (2H, d, J=7.5 Hz), 7.71 (1H, d, J=7.5 Hz), 7.57 (2H, d, J=7.5 Hz), 7.49 (1H, d, J=7.9 Hz), 7.44 (1H, s), 7.33 (2H, m), 7.18 (1H, d, J=7.9 Hz), 4.28 (2H, d, J=5.6 Hz), 3.50.(1H, s). MS: 362.4 (M+1); 360.2 (M−1).

### Example 15

### Synthesis of N-hydroxy-4-{[3-(3-phenylpropylamino)methyl]phenyl}benzamide

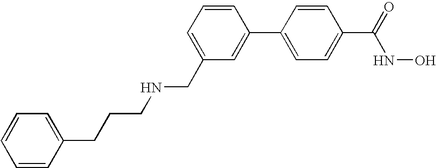 Step 1

To a prestirred (30 min) solution of 4-bromo-benzoic acid ethyl ester (2.8 g, 12.3 mmol) and tetrakis(triphenylphosphine)palladium(0) (0.42 g, 0.37 mmol) in toluene (50 ml) and ethanol (10 ml) was added 3-formylphenylboronic acid (2.0 g, 13.5 mmol) and K₂CO₃ (8.5 g, 61 mmol). The resulting slurry was heated to 75° C. for 3 h then cooled to room temperature and diluted with ethyl acetate (200 ml) and washed with water (300 ml). The organic layer was dried (MgSO₄), filtered and concentrated to a brown oil. Flash chromatography (20% ethyl acetate/hexane) provided 1.6 g (51%) of 3′-formyl-biphenyl-4-carboxylic acid ethyl ester as a white solid.

Step 2

To a prestirred solution (1 h) of 3′-formyl-biphenyl-4-carboxylic acid ethyl ester (0.20 g, 0.83 mmol) and 3-phenyl-propylamine (0.1 g, 0.83 mmol) in methanol (10 ml) was added NaBH₄ (32 mg, 0.83 mmol). After stirring 3 h at room temperature, the solution was diluted with ethyl acetate (100 ml) and washed with water (200 ml). The organic layer was dried (MgSO₄), filtered and concentrated to collect 0.29 g (97%) of crude 3′-[(3-phenyl-propylamino)-methyl]-biphenyl-4-carboxylic acid ethyl ester.

Step 3

To a solution of crude 3′-[(3-phenyl-propylamino)-methyl]-biphenyl-4-carboxylic acid ethyl ester (30.29 g, 0.81 mmol) and hydroxylamine (1.5 ml, 50% aqueous solution) in methanol (7 ml) was added NaOH (65 mg, 1.6 mmol) dissolved in water (1 ml). After 4 h of stirring at room temperature, the reaction solution was concentrated under vacuum and the resulting residue was subjected to reverse phase HPLC to provide 178 mg (55%) of the title compound as the HCl salt.

¹H NMR (DMSO-d₆): 9.46 (2H, br s), 9.07 (1H, br s), 8.00 (1H, s), 7.86 (2H, d, J=8.4 Hz), 7.78 (2H, d, J=8.4 Hz), 7.74 (1H, d, J=7.5 Hz), 7.52 (2H, m), 7.25 (2H, m), 7.18 (3H, m), 4.18 (2H, s), 2.89 (2H, s), 2.64 (2H, t, J=7.4 Hz), 1.99 (2H, m). MS: 361.4 (M+1); 359.3 (M−1).

Proceeding as described in Example 15 above, but using suitable starting materials the following compounds were prepared.

N-hydroxy-4-[3-(benzylamino-methyl)phenyl]benzamide. ¹H NMR (DMSO-d₆): 9.06 (1H, bs), 7.82 (2H, d, J=8.3 Hz), 7.73 (2H, d, J=8.3 Hz), 7.67 (1H, s), 7.55 (1H, d, J=7.6 Hz), 7.32 (7H, m), 3.75 (2H, s), 3.70 (2H, s); MS: 333.1 (M+1).

N-hydroxy-4-{3-{[2-(1H-Indol-3-yl)-ethyl]aminomethyl}phenyl}benzamide. ¹H NMR (DMSO-d₆): 11.22 (1H, s), 10.88 (1H, s), 9.23 (1H, bs), 7.91 (1H, s), 7.80 (2H, d, J=7.7 Hz), 7.71 (3H, m), 7.47 (3H, m), 7.27 (1H, d, J=8.4 Hz), 7.15 (1H, s), 7.00 (1H, t, J=7.3 Hz), 6.90 (1H, t, J=7.3 Hz), 4.20 (2H, s), 3.09 (4H, m); MS: 386.0 (M+1).

N-hydroxy--4-{3-[(3,4-methylenedioxyphenyl)methylaminomethyl]phenyl}benzamide. ¹H NMR (DMSO-d₆): 9.05 (1H, bs), 7.82 (2H, d, J=8.1 Hz), 7.73 (2H, d, J=8.1 Hz), 7.65 (1H, s), 7.55 (1H, d, J=7.6 Hz), 7.38 (1H, t, J=7.6 Hz), 7.34 (1H, d, J=7.6 Hz), 6.93 (1H, s), 6.80 (2H, m), 5.95 (2H, s), 3.71 (2H, s), 3.61 (2H, s); MS: 374.7 (M−1), 376.9 (M+1).

N-hydroxy-4-[3-(pyridin-4-ylmethylaminomethyl)phenyl]benzamide. ¹H NMR (DMSO-d₆): 11.22 (1H, bs), 10.12 (1H, bs), 8.73 (2H, d, J=4.7 Hz), 7.95 (1H, s), 7.86 (2H, d, J=5.3 Hz), 7.78 (2H, d, J=8.0 Hz), 7.70 (3H, m), 7.45 (2H, m), 4.32 (2H, s), 4.22 (2H, s); MS: 334.0 (M+1).

N-hydroxy-4-{3-{[2-(pyridin-3-yl)-ethyl]aminomethyl}phenyl}benzamide. ¹H NMR (DMSO-d₆): 11.23 (1H, bs), 9.63 (1H, bs), 8.78 (1H, s), 8.69 (1H, d, J=5.4 Hz), 8.33 (1H, d, J=8.4 Hz), 7.96 (1H, s), 7.84 (1H, t, J=7.2 Hz), 7.79 (2H, d, J=7.8 Hz), 7.73 (2H, d, J=7.8 Hz), 7.68 (1H, d, J=7.2 Hz), 7.47 (2H, m), 4.17 (2H, s), 3.23 (4H, m); MS: 346.3 (M−1), 348.0 (M+1).

N-hydroxy-4-{3-[N-benzoyl-N-(2-hydroxylethyl)aminomethyl]phenyl}benzamide. ¹H NMR (DMSO-d₆): 11.20 (1H, s), 8.99 (1H, s), 7.78-7.54 (6H, m), 7.42-7.30 (7H, m), 4.74 (2H, br s), 4.55 (1H, br s), 3.39 (2H, br s), 3.26 (2H, s). MS: 391.2 (M+1).

N-hydroxy-4-{3-[N-benzyl-N-(2-hydroxylethyl)aminomethyl]phenyl}benzamide. ¹H NMR (DMSO-d₆): 10.94 (1H, s), 8.07 (1H, s), 7.86-7.77 (5H, m), 7.64 (3H, m), 7.53 (1H, t, J=7.6 Hz), 7.43 (3H, m), 4.41 (4H, m), 3.81 (2H, t, J=4.5 Hz), 3.04 (2H, s). MS: 376.9 (M+1).

N-hydroxy-4-{3-[N-benzyl-N-(2-methoxyethyl)aminomethyl]phenyl}benzamide. ¹H NMR (DMSO-d₆): 11.25 (1H, s), 8.09 (1H, s), 7.87-7.77 (5H, m), 7.65 (3H, m), 7.52 (1H, t, J=7.7 Hz), 7.43 (3H, m), 4.38 (4H, m), 3.74 (2H, s), 3.23 (3H, s), 3.15 (2H, s). MS: 391.0 (M+1).

N-hydroxy-4-[3-(phenylaminomethyl)phenyl]benzamide. ¹H NMR (DMSO-d₆): 11.27 (1H, bs), 7.83 (2H, dd, J=8.4 Hz, 1.6 Hz), 7.77 (1H, s), 7.72 (2H, dd, J=8.4 Hz, 1.6 Hz), 7.60 (1H, d, J=6.8 Hz), 7.42 (2H, m), 7.15 (2H, m), 6.80 (3H, m), 4.41 (2H, s); MS: 318.8 (M+1), 316.8 (M−1)

N-hydroxy-4-[3-(4-methylphenylaminomethyl)phenyl]benzamide. ¹H NMR (DMSO-d₆): 11.23 (1H, bs), 7.82 (2H, d, J=8.4 Hz), 7.71 (3H, m), 7.59 (1H, d, 7.4 Hz), 7.40 (2H, m), 6.96 (2H, bs), 6.73 (2H, bs), 4.34 (2H, s), 2.15 (3H, s); MS: 333.1 (M+1), 331.3 (M−1)

N-hydroxy-4-[3-(4-chlorophenylaminomethyl)phenyl]benzamide. ¹H NMR (DMSO-d₆): 11.25 (1H, s), 7.82 (2H, dd, J=8.3 Hz, 1.6 Hz), 7.70 (3H, m), 7.56 (1H, d, J=7.1 Hz), 7.41 (1H, td, J=7.4 Hz, 1.2 Hz), 7.35 (1H, d, J=7.1 Hz), 7.05 (2H, dd, J=8.3 Hz, 1.6 Hz), 6.60 (2H, d, J=7.6 Hz), 4.32 (2H, s); MS: 353.1 (M+1), 351.1 (M−1).

### Example 16

### Synthesis of N-hydroxy-4-(3-pyridin-3-yloxymethylphenyl)benzamide

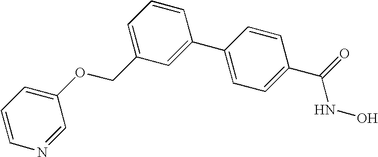 Step 1

To a solution of 3′-formyl-biphenyl-4-carboxylic acid ethyl ester (0.20 g, 0.80 mmol) in methanol (10 ml) was added NaBH₄ (30 mg, 0.80 mmol). After stirring 3 h at room temperature, the solution was diluted with ethyl acetate (75 ml) and washed with water (200 ml). The organic layer was dried (MgSO₄), filtered and concentrated. The resulting residue was subjected to flash chromatography (25% ethyl acetate/hexane) provided 0.16 g (78%) of 3′-hydroxymethyl-biphenyl-4-carboxylic acid ethyl ester.

Step 2

To a solution of 3′-hydroxymethyl-biphenyl-4-carboxylic acid ethyl ester (0.15 g, 0.59 mmol), 3-hydroxypyridine (67 mg, 0.70 mmol) and triphenylphosphine (0.18 g, 0.70 mmol) in THF (5 ml) was added diethylazodicarboxylate (0,11 ml, 0.70 mmol). After stirring 1.5 h at room temperature, the solution was diluted with ethyl acetate (75 ml) and washed with mild NaHCO₃ (100 ml). The organic layer was dried (MgSO₄), filtered and concentrated and the residue was subjected to flash chromatography (50% ethyl acetate/hexane) to provide 0.20 g (˜100%) of 3′-(pyridin-3-yloxymethyl)-biphenyl-4-carboxylic acid ethyl ester as a light yellow solid.

Step 3

To a solution of 3′-(pyridin-3-yloxymethyl)-biphenyl-4-carboxylic acid ethyl ester (0.20 g, 0.60 mmol) in methanol (3 ml) and TBF (3 ml) was added NaOH (0.3 g dissolved in 1 ml of H₂O). The solution was stirred 2 h at room temperature, then diluted with water (50 ml) and the pH was adjusted to ˜4-5 with 1N HCl. The aqueous layer was then extracted with ethyl acetate (2×75 ml). The organic layer was dried (MgSO₄), filtered and concentrated to give 3′-(pyridin-3-yloxymethyl)-biphenyl-4-carboxylic acid which was taken further without further purification.

Step 4

3′-(Pyridin-3-yloxymethyl)-biphenyl-4-carboxylic acid was stirred in THF (3 ml) with 2 drops of DMF, then oxalyl chloride was added (0.1 mL). The solution was stirred 30 minutes at room temperature then concentrated. The residue was then stirred in THF (2 ml) and to this was added hydroxylamine (50% aq. solution). After an additional 30 minutes of stirring at room temperature, the solvent was removed under vacuum and the resulting residue was subjected to reverse phase HPLC to provide 17 mg of the title compound.

¹H NMR (DMSO-d₆): 9.05 (1H, s), 8.37 (1H, s), 8.15 (1H, d, J=4.6 Hz), 7.82 (3H, m), 7.74 (2H, d, J=7.8 Hz), 7.67 (1H, d, J=6.9 Hz), 7.48 (3H, m), 7.33 (1H, m), 5.25 (2H, s). MS: 321.0 (M+1); 319.1 (M−1).

Proceeding as described in Example 16 above, but using suitable starting materials the following compounds were prepared.

N-hydroxy-4-[3-(phenyloxymethyl)phenyl]benzamide: ¹H NMR (DMSO-d₆): 11.81 (1H, s), 8.97 (1H, s), 7.77 (2H, d, J=8.7 Hz), 7.69 (1H, s), 7.67 (2H, d, J=8.7 Hz), 7.59 (1H, d, J=7.0 Hz), 7.42 (2H, m), 7.22 (2H, m), 6.95 (2H, d, J=8.7 Hz), 6.85 (1H, t, 7.0 Hz), 5.01 (2H, s); MS: 318.2 (M−1), 320.1 (M+1).

N-hydroxy-4-[3-(4-methylphenyloxymethyl)phenyl]benzamide. ¹H NMR (DMSO-d₆): 11.26 (1H, s), 9.05 (1H, s), 7.83 (2H, d, J=7.5 Hz), 7.74 (3H, m), 7.65 (1H, d, J=7.5 Hz), 7.47 (2H, m), 7.07 (2H, d, J=7.5 Hz), 6.91 (2H, d, J=7.5 Hz), 5.13 (2H, s), 2.22 (3H, s); MS: 332.3 (M−1), 334.1 (M+1).

N-hydroxy-4-[3-(4-chlorophenyloxymethyl)phenyl]benzamide. ¹H NMR (DMSO-d₆): 11.26 (1H, s), 9.05 (1H, s), 7.83 (2H, d, J=7.9 Hz), 7.75 (3H, m), 7.67 (1H, d, J=7.1 Hz), 7.47 (2H, m), 7.32 (2H, d, J=7.9 Hz), 7.05 (2H, d, J=7.9 Hz), 5.17 (2H, s); MS: 352.1 (M−1), 354.0 (M+1).

### Example 17

### Synthesis of N-hydroxy-4-(3-benzylaminophenyl)benzamide

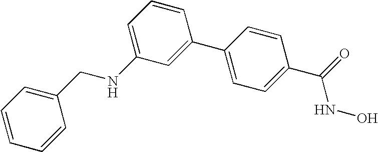 Step 1

To a prestirred (1 h) solution of 3′-amino-biphenyl-4-carboxylic acid methyl ester (0.20 g, 0.88 mmol) and benzaldehyde (0.09 ml, 0.84 mmol) in methanol (5 ml) was added NaBH₄ (30 mg, 0.84 mmol). After stirring 1 h at room temperature, the solution was diluted with ethyl acetate (75 ml) and washed with mild NaHCO₃ (100 ml). The organic layer was dried (MgSO₄), filtered and concentrated to collect crude 3′-benzylamino-biphenyl-4-carboxylic acid methyl ester.

Step 2

To a solution of crude 3′-benzylamino-biphenyl-4-carboxylic acid methyl ester from above and hydroxylamine (2.8 ml, 50% aqueous solution) in methanol (7 ml) was added NaOH (90 mg, 2.3 mmol) dissolved in water (1 ml). After 4 h of stirring at room temperature, the reaction solution was concentrated under vacuum and the resulting residue was subjected to reverse phase HPLC to provide 100 mg (32%) of the title compound as the HCl salt.

¹H NMR (DMSO-d₆): 11.24 (1H, bs), 7.79 (2H, d, J=8.7 Hz), 7.60 (2H, d, J=8.7 Hz), 7.40 (2H, d, J=7.1 Hz), 7.31 (2H, m), 7.22 (2H, m), 7.06 (1H, s), 7.02 (1H, bs), 6.78(1H, bs), 4.39 (2H, s); MS: 319.1 (M+1), 317.3 (M−1).

Proceeding as described in Example 17 above, but using suitable starting materials the following compounds were prepared.

N-hydroxy-4-[(3-methoxybenzylamino)phenyl]benzamide. ¹H NMR (DMSO-d₆): 11.19 (1H, s), 9.01 (1H, s), 7.77 (2H, d, J=8.6 Hz), 7.58 (2H, d, J=8.6 Hz), 7.26 (2H, d, J=9.1 Hz), 7.11 (1H, t, J=8.0 Hz), 6.84 (4H, m), 6.80 (1H, d, J=7.4 Hz), 6.58 (1H, d, J=7.4 Hz), 6.29 (1H, bs), 4.24 (2H, s), 3.70 (3H, s); MS: 349.4 (M+1), 346.9 (M−1).

N-hydroxy-4-[3-(4-methylbenzylamino)phenyl]benzamide. ¹H NMR (DMSO-d₆): 7.76 (2H, d, J=8.8 Hz), 7.57 (2H, d, J=8.8 Hz), 7.24 (2H, d, J=8.2 Hz), 7.11 (3H, m), 6.81 (2H, m), 6.57 (1H, d, J=8.2 Hz), 6.33 (1H, t, J=6.3 Hz), 4.26 (2H, d, J=6.3 Hz), 2.25 (3H, s); MS: 333.1 (M+1), 331.2 (M−1).

### Example 18

### Synthesis of N-hydroxy-4-[3-(phenylsulfonylamino)-5-(propoxymethyl)-phenyl]benzamide

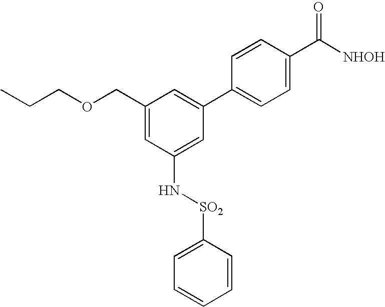 Step 1

3-Nitrobenzaldehyde (15.1 g, 100 mmol) and N-bromosuccinamide (18.7 g, 105 mmol) were mixed in TFA (80 mL) and sulfuric acid (20 mL) and the reaction mixture was refluxed under N₂(g) for 24 h. After cooling, the reaction mixture was poured into ice water with stirring. The precipitates were collected, washed with water and dried to give 17 g of 3-bromo-5-nitrobenzaldehyde.

Step 2

To a solution of 3-bromo-5-nitrobenzaldehyde (10.2 g, 44.3 mmol) in THF was added sodium borohydride (0.84 g, 22.2 mmol) in portions over 15 mins. After 2-3 h, water was slowly add to destroy excess unreacted sodium borohydride and the reaction mixture was acidified to pH 3 with dilute HCl(aq) and extracted with EtOAc. The organic layer was separated, dried, and concentrated to give 8.9 g (crude) of 3-bromo-5-nitrobenzyl alcohol.

Step 3

Sodium hydride (89 mg, 3.7 mmol) was dissolved in anhydrous DMF and stirred under N₂(g). A solution of 3-bromo-5-nitrobenzyl alcohol (719 mg, 3.1 mmol) in anhydrous DMF was added dropwise and the reaction mixture was stirred for 30 mins. Iodopropane (333 μL, 3.4 mmol) was added and after stirring for 12 h water was slowly added to destroy excess of sodium hydride. The reaction mixture was diluted with EtOAc and washed with dilute HCl(aq). The organic layer was separated, dried, and concentrated. Purification of the crude product on a silica gel column (20% EtOAc in hexane) gave 473 mg of 1-bromo-3-nitro-5-propoxymethylbenzene.

Step 4

To a solution of 1-bromo-3-nitro-5-propoxymethylbenzene (473 mg, 1.7 mmol) in dioxane (20 ml) was added (4-methoxycarbonylphenyl)boronic acid (301 mg, 1.8 mmol), potassium carbonate (716 mg, 5.18 mmol), EtOH (5 mL) and tetrakis(triphenylphosphine)-palladium(0) (100 mg, 0.86 mmol) and the reaction mixture was refluxed under N₂(g) for 4-5 h. After cooling, the reaction mixture to room temperature NaOH(aq) and dioxane were added and the reaction mixture was filtered through celite. The filtrate was washed with diethyl ether, the aqueous portion was acidified to pH 2 with dilute HCl(aq) and extracted with diethyl ether. The organic layer was separated, dried and concentrated to give 527 mg of 3′-nitro-5′-propoxymethylbiphenyl-4-carboxylic acid.

Step 5

3′-Nitro-5′-propoxymethylbiphenyl-4-carboxylic acid (527 mg, 1.67 mmol) was dissolved in anhydrous DMF and cesium carbonate (545 mg, 1.67 mmol) and iodomethane (125 μL, 2 mmol) were added. After stirring for 30-60 mins., the reaction mixture was diluted with EtOAc and washed with aqueous sodium bicarbonate solution and dilute HCl(aq). The organic layer was separated, dried and concentrated. The crude product was purified on a silica gel column (20% EtOAc in hexane) to give 244 mg of 3′-nitro-5′-propoxymethyl-biphenyl-4-carboxylic acid methyl ester.

Step 6

3′-Nitro-5′-propoxymethylbiphenyl-4-carboxylic acid methyl ester (244 mg, 0.74 mmol) was dissolved in 20 ml of 1:1 MeOH/HOAc. Fe(0) dust was added and the reaction mixture was refluxed under N₂(g) for 4 h. After cooling, the reaction mixture was diluted with EtOAc and washed with aqueous sodium carbonate solution. The organic layer was separated, dried and concentrated. Purification on a silica gel column (40% EtOAc in hexane) gave 132 mg 3′-amino-5′-propoxymethylbiphenyl-4-carboxylic acid methyl ester.

Step 7

3′-Amino-5′-propoxymethylbiphenyl-4-carboxylic acid methyl ester (132 mg, 0.441 mmol) was dissolved in anhydrous DMF and benzenesulfonyl chloride (62 μL, 0.485 mmol) and triethylamine(92 μL, 0.662 mmol) were added. After 1-2 h, the reaction mixture was diluted with EtOAc and washed with saturated NaCl(aq). The organic layer was separated and concentrated and the crude product was dissolved in MeOH and added NaOH(aq) was added till pH 12-13. The reaction mixture was stirred overnight. The reaction mixture was acidified to pH 2 and extracted with EtOAc. The organic layer was separated and concentrated to give 3′-phenylsulfonylamino-5′-propoxymethylbiphenyl-4-carboxylic acid.

Step 8

3′-Phenylsulfonylamino-5′-propoxymethylbiphenyl-4-carboxylic acid (100 mg, 0.235 mmol) was dissolved in anhydrous DMF and HOBt (79 mg, 0.588 mmol) and EDC (90 mg, 0.471 mmol) were added. After 1-2 h, hydroxylamine hydrochloride (50 mg, 0.705 mmol) and TEA (98 μL, 0.705 mmol) were added and stirring was continued for 1-2 h. The reaction mixture was diluted with EtOAc and washed with dilute HCl(aq). After concentration, the crude was purified with prep HPLC to give the title compound (29 mg).

¹H NMR (DMSO-d₆) δ 11.253 (s, 1H), 10.454 (s, 1H), 7.803 (m, 4H), 7.544 (m, 5H), 7.279 (m, 2H), 7.090 (s, 1H), 4.411 (s, 2H), 3.307 (t, J=6.8 Hz, 2H), 1.528 (q, J=5.2 Hz, 2H), 0.868 (t, J=7.6 Hz, 3H). Mass, ESI: (M⁺+H): 441.5 (calc.); 441.2 (obs.)

Proceeding as described in Example 18 above, but using suitable starting materials the following compounds were prepared.

N-hydroxy-4-[3-(4-methoxyphenylcarbonylamino)-5-(propoxymethyl)-phenyl]benzamide. ¹H NMR (DMSO-d₆) δ 11.266 (s, 1H), 10.192 (s, 1H), 9.061 (s, 1H), 8.070 (s, 1H), 7.978 (d, J=9.2 Hz, 2H), 7.855 (d, J=7.2 Hz, 2H), 7.809 (s, 1H), 7.705 (d, J=7.2 Hz, 2H), 4.525 (s, 2H), 3.843 (s, 3H), 3.438 (t, J=6.4 Hz, 2H), 1.590 (q, J=8 Hz, 2H), 0.914 (t, J=7.2 Hz, 3H).

Mass, ESI: (M⁺+H): 435.5 (calc.); 435.1 (obs.).

N-hydroxy-4-[3-(isopropylcarbonylamino)-5-(propoxymethyl)-phenyl]benzamide. ¹H NMR (DMSO-d₆) δ 11.258 (s, 1H), 9.948 (s, 1H), 9.056 (s, 1H), 7.924 (s, 1H), 7.839 (d, J=7.6 Hz, 2H), 7.662 (d, J=7.6 Hz, 2H), 7.588 (s, 1H), 7.286 (s, 1H), 4.489 (s, 2H), 3.420 (t, J=6.4 Hz, 2H), 2.610 (m, 1H), 1.558 (q, J=8 Hz, 2H), 1.113 (d, J=6.4 Hz, 6H), 0.914 (t, J=7.2 Hz, 3H). Mass, ESI: (M⁺+H): 371.4 (calc.); 370.9 (obs.)

N-hydroxy 4-[3-(phenylureido)-5-(propoxymethyl)-phenyl]benzamide. ¹H NMR (DMSO-d₆) δ 11.264 (s, 1H), 9.057 (s, 1H), 8.825 (s, 1H), 8.701 (s, 1H), 7.845 (d, J=7.2 Hz, 2H), 7.752 (s, 1H), 7.684 (d, J=7.2 Hz, 2H), 7.397 (s, 1H), 7.254 (m, 3H), 6.965 (t, J=7.2 Hz, 1H), 4.503 (s, 2H), 3.430 (t, J=6.4 Hz, 2H), 1.586 (q, J=8 Hz, 2H), 0.918 (t, J=7.2 Hz, 3H). Mass, ESI: (M⁺+H): 420.5 (calc.); 420.3 (obs.)

N-hydroxy-4-[3-(benzylamino)-5-(propoxymethyl)-phenyl]benzamide. ¹H NMR (DMSO-d₆) δ 7.785 (d, J=8 Hz, 2H), 7.590 (d, J=6.8 Hz, 2H), 7.386 (d, J=7.2 Hz, 2H), 7.313 (t, J=7.2 Hz, 2H), 7.223 (m, 1H), 6.872 (bs, 2H), 6.693 (bs, 1H), 4.378 (s, 4H), 3.344 (t, J=6.4 Hz, 2H), 1.524 (q, J=8 Hz, 2H), 0.870 (t, J=7.2 Hz, 3H). Mass, ESI: (M⁺+H): 391.5 (calc.); 391.2 (obs.)

N-hydroxy-4-{3-[(2-phenylethenyl)carbonylamino]-5-(propoxymethyl)-phenyl}benzamide. ¹H NMR (DMSO-d₆) δ 11.272 (s, 1H), 10.457 (s, 1H), 9.057 (s, 1H), 8.016 (s, 1H), 7.856 (d, J=7.2 Hz, 2H), 7.609 (m, 6H), 7.447 (m, 3H), 7.328 (s, 1H), 6.892 (d, J=15.6 Hz, 1H), 4.524 (s, 2H), 3.442 (t, J=6.4 Hz, 2H), 2.610 (m, 1H), 1.593 (q, J=8 Hz, 2H), 0.920 (t, J=7.2 Hz, 3H). Mass, ESI: (M⁺+H): 431.5 (calc.); 430.9 (obs.)

N-hydroxy-4-[3-(phenylaminomethylcarbonylamino)-5-(propoxymethyl)-phenyl]benzamide. ¹H NMR (DMSO-d₆) δ 10.149 (s, 1H), 7.903 (s, 1H), 7.828 (d, J=8 Hz, 2H), 7.646 (m, 3H), 7.309 (s, 1H), 7.102 (t, J=7.2 Hz, 2H), 6.626 (m, 3H), 4.491 (s, 2H), 3.897 (s, 2H), 3.412 (t, J=6.4 Hz, 2H), 1.570 (q, J=8 Hz, 2H), 0.899 (t, J=7.2 Hz, 3H). Mass, ESI: (M⁺+H): 434.5 (calc.); 434.3 (obs.)

N-hydroxy-4-[3-(benzyloxymethyl)-5-(4-methoxyphenylcarbonylamino)-phenyl]benzamide. ¹H NMR (DMSO-d₆) δ 11.265 (s, 1H), 10.207 (s, 1H), 8.098 (s, 1H), 7.985 (d, J=7.2 Hz, 2H), 7.863 (m, 3H), 7.715 (d, J=6.8 Hz, 2H), 7.383 (m, 5H), 7.307 (m,1H), 7.065 (d, J=8 Hz, 2H), 4.616 (s, 2H), 4.593 (s, 2H), 3.845 (s, 3H). Mass, ESI: (M⁺+H): 483.5 (calc.); 483.1 (obs.)

N-hydroxy-4-[3-(4-methoxyphenylcarbonylamino)-5-(phenyloxymethyl)-phenyl]benzamide. ¹H NMR (DMSO-d₆) δ 11.272 (s, 1H), 10.236 (s, 1H), 8.109 (s, 1H), 7.985 (d, J=6.8 Hz, 2H), 7.935 (s, 1H), 7.867 (d, J=6.4 Hz, 2H), 7.728 (d, J=6.4 Hz, 2H), 7.516 (s, 1H), 7.298 (t, J=6.8 Hz, 2H), 7.055 (m, 4H), 6.938 (t, J=7.6 Hz, 1H), 5.180 (s, 2H), 3.844 (s, 3H). Mass, ESI: (M⁺+H): 469.5 (calc.); 469.3 (obs.)

N-hydroxy-4-[3-(4-methoxyphenylcarbonylamino)-5-(3-phenylprop-2-enyloxymethyl)-phenyl]benzamide. ¹H NMR (DMSO-d₆) δ 11.267 (s, 1H), 10.206 (s, 1H), 9.058 (s, 1H), 8.090 (s, 1H), 7.987 (d, J=8 Hz, 2H), 7.865 (m, 3H), 7.720 (d, J=8 Hz, 2H), 7.464 (d, J=8 Hz, 2H), 7.398 (s, 1H), 7.323 (t, J=6.4 Hz, 2H), 7.237 (m,1H), 7.066 (d, J=8 Hz, 2H), 6.711 (d, J=15.6 Hz, 1H), 6.458 (m, 1), 4.612 (s, 2H), 4.228 (d, J=5.6 Hz, 2H), 3.845 (s, 3H). Mass, ESI: (M⁺+H): 509.6 (calc.); 509.1 (obs.).

### Example 19

### Synthesis of N-hydroxy-4-(3-[(2,4-dichlorophenyl)carbonylamino]-5-(dimethylaminomethyl)-phenyl)benzamide

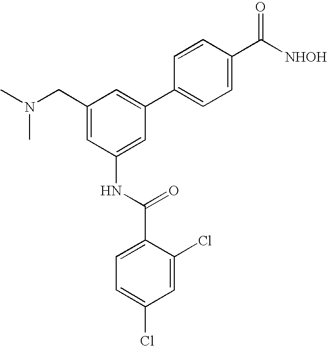 Step 1

To a solution of 3-nitrobenzaldehyde (15.00 g, 99.2 mmol) in a mixture of trifluoroacetic acid (80 ml) and sulfuric acid (20 ml) was added N-bromosuccinimide (18.6 g, 104.5 mmol) protionwise. The reaction mixture was heated to reflux under a nitrogen balloon overnight. The reaction mixture was poured into ice and the resulting pale yellow precipitate was collected by vacuum filtration and air-dried to afford 3-bromo-5-nitrobenzaldehyde (16.0 g, 70% yield).

Step 2

To a solution of the 3-bromo-5-nitrobenzaldehyde (1.37 g, 5.96 mmol) in a mixture of toluene (20 ml) and methanol (10 ml) was added 4-methoxycarbonyl boronic acid (1.18 g, 6.56 mmol). To this was added potassium carbonate (3.70 g, 26.8 mmol) and the reaction flask was flushed with nitrogen. Tetrakis(triphenylphosphine)palladium(0) (0.34 g, 0.30 mmol) was added and the reaction mixture heated to reflux overnight. The reaction mixture was diluted with ethyl acetate and washed 5% with citric acid, followed by brine. The organic layers were combined, dried over anhydrous sodium sulfate and concentrated to afford 3′-formyl-5′-nitrobiphenyl-4-carboxylic acid methyl ester a brown solid (10.10 g, 65% yield).

Step 3

To a solution of 3′-formyl-5′-nitrobiphenyl-4-carboxylic acid methyl ester (0.50 g, 1.75 mmol) and dimethylamine hydrochloride (0.14 g, 1.75 mmol) in 1,2-dichloroethane (5 ml) and methanol (10 ml) was added triethylamine (0.33 ml, 2.34 mmol) followed by sodium triacetoxyborohydride (0.52 g, 2.45 mmol). The reaction mixture was stirred under nitrogen overnight. Ethyl acetate was added and the solution extracted with ethyl acetate/water. The organic layer was washed with brine and dried over anhydrous sodium sulfate. Concentration afforded 3′-dimethylaminomethyl-5′-nitrobiphenyl-4-carboxylic acid methyl ester as an oil (0.40 g, 73% yield).

Step 4

To a solution of the 3′-dimethylaminomethyl-5′-nitrobiphenyl-4-carboxylic acid methyl ester (0.36 g, 1.26 mmol) in EtOH (15 ml) under an atmosphere of nitrogen was added 10% palladium on carbon (25 mgs). The reaction mixture was stirred under a ballon on hydrogen overnight. The catalyst was removed by filtration and the ethanol removed to yield 5′-amino-3′-dimethylaminomethylbiphenyl-4-carboxylic acid methyl ester as a colorless oil (0.34 g, 87% yield). MS: calc314.13; found 315.5(M+1).

Step 5

To a solution of 5′-amino-3′-dimethylaminomethylbiphenyl-4-carboxylic acid methyl ester (0.34 g, 1.20 mmol) in DCM was added DIEA (0.38 ml, 2.16 mmol) followed by 2,4-dichlorobenzoyl chloride (0.18 ml, 1.32 mmol). The reaction was stirred under nitrogen overnight. The reaction mixture was diluted with ethyl acetate, washed with water and brine. The combined organic layers were dried over anhydrous sodium sulfate and concentrated to afford 3′-dimethylaminomethyl-5′-phenylcarbonylaminobiphenyl-4-carboxylic acid methyl ester as a tan colored solid (0.50 g, 91% yield). MS: calc 456.1; found 457.5(M+1).

Step 6

To a solution of 3′-dimethylaminomethyl-5′-phenylcarbonylaminobiphenyl-4-carboxylic acid methyl ester (0.50 g, 1.09 mmol) in a mixture of THF and methanol (2 mls) in an ice bath was added 50% hydroxylamine (2.5 m]). The reaction mixture was stirred for 15 min., and then a solution of sodium hydroxide (0.18 g, 4.36 mmol) in methanol (2 mls). The reaction mixture was stirred overnight. The solvents were removed under reduced pressure and the resulting aquesous oil extracted with ethyl acetate. The ethyl acetate was washed with brine and dried over anhydrous sodium sulfate. Concentration afforded an oil which was purified by reverse phase HPLC to afford the title compound as a pale pink powder (38 mgs, 8% yield). ¹H NMR(DMSO-d6): 11.29 (s, 1H), 10.49 (s, 1H), 8.10-7.79 (m, 5H), 7.59-7.43 (m, 4H), 7.02 (s, 1H), 3.6 (s, 2H), 2.3 (m, 6H); MS: calc 458.36; found 457.2 (M−1), 459.7 (M+1).

Proceeding as described above, but substituting appropriate starting materials the following compounds were prepared.

N-hydroxy-4-[3-(4-methylphenylcarbonylamino)-5-(dimethylaminomethyl)-phenylibenzamide: ¹H NMR(DMSO-d6): 11.29 (s, 1H), 10.53 (s, 1H), 8.49 (s, 1H), 8.42 (s, 1H), 8.02-7.53 (m, 10H); MS: calc 376.37; found 374.8 (M−1), 377.2 (M+1).

N-hydroxy-4-[3-(2,4-dichlorophenylcarbonylaino)-5-(piperidin-1-ylmethyl)-phenyl]benzamide: ¹H NMR(DMSO-d6): 11.28 (s, 1H), 10.45 (s, 1H), 8.18 (br t, 1H), 7.99-7.44 (m, 11H), 3.00 (br d, 6H); MS: calc 403.4; found 402.0 (M−1), 404.2 (M+1).

N-hydroxy-4-{3-[(2,4-dichlorophenyl)carbonylamino]-5-(pyrrolidin-1-ylmethyl)-phenyl}benzamide: ¹H NMR(DMSO-d6): 11.30 (s, 1H), 10.52 (s, 1H), 10.46 (s, 1H), 8.46 (m, 1H), 8.39 (m, 1H), 8.20 (m, 1H), 7.99-7.44 (m, 12H), 3.62 (br s 2, 8H); MS: calc 445.5; found 444.4 (M−1), 444.6 (M+1).

N-hydroxy-4-[3-(2,4-dichlorobenzylamino)-5-(dimethylaminocarbonyl)-phenyl]benzamide: ¹H NMR(DMSO-d6): 10.37 (s, 1H), 8.20 (s, 1H), 7.91-7.73 (m, 8H), 7.44-7.34 (m, 2H), 3.62 (br 2, 8H), 2.38 (s, 3H); MS: calc 459.5; found 458.4 (M−1), 460.6 (M+1).

N-hydroxy-4-[3-(dimethylaminomethyl)-5-(furan-3-ylcarbonylamino)-phenyl]benzamide: ¹H NMR (CD₃OD): 8.27 (m,1H), 8.04 (m,1H), 7.96 (m,1H), 7.89-7.86 (d, 2H, J=8 Hz), 7.81-7.76 (d, 2H, J=8 Hz), 7.64 (m, 1H), 7.60 (m, 1H), 6.97 (m,1H), 4.41 (s, 2H), 2.94 (s, 6H);MS: calc 379.15; found 380.1 (M+1), 378.0 (M−1).

### Example 20

### Synthesis of acetyl-Gly-Ala-(N-acetyl-Lys)-AMC

tert-Boc (N-Acetyl-Lys)-AMC (445 mg, 1 mmol, purchased from Bachem) was dissolved in 4 M HCL in dioxane to provide H-(N-acetyl-Lys)-AMC as a white solid. To a solution of H-(N-acetyl-Lys)-AMC in DMF (5 ml) was added Ac-Gly-Ala-OH (188 mg, 1 mmol) using PyBOP (520 mg, 1 mmol), HOBt (135 mg, 1 mmol), and NMM (0.296 ml, 2 mmol). The reaction mixture was stirred for 1 h and monitored by MS/LC for the presence of H-(N-acetyl-Lys)-AMC. Additional amounts of PYBOP (260 mg, 0.5 mmol), HOBt (70 mg, 0.5 mmol), and NMM (0.146 ml, 1 mmol) was added and the stirring was continued for additional 4 h after which the product was isolated in quantative yield.

### BIOLOGICAL EXAMPLES

### Example 1

### Inhibition of HDAC In Vitro

The HDAC inhibitory activity of the compounds of this invention in vitro was determined as follows.

Measurements were performed in a reaction volume of 100 μL using 96-well assay plates. HDAC-1 (200 pM final concentration) in reaction buffer (50 mM HEPES, 100 mM KCl, 0.001% Tween-20, 5% DMSO, pH 7.4) was mixed with inhibitor at various concentrations and allowed to incubate for 30 minutes, after which trypsin and acetyl-Gly-Ala-(N-acetyl-Lys)-AMC were added to final concentrations of 50 nM and 25 μM, respectively, to initiate the reaction. Negative control reactions were performed in the absence of inhibitor in replicates of eight.

The reactions were monitored in a fluorescence plate reader. After a 30 minute lag time, the fluorescence was measured over a 30 minute time frame using an excitation wavelength of 355 nm and a detection wavelength of 460 nm. The increase in fluorescence with time was used as the measure of the reaction rate. Inhibition constants were obtained using the program BatchKi (Kuzmic et al. Anal. Biochem. 2000, 286, 45-50).

### Example 2

### Cell Proliferation Assay In Vitro

The ability of the compounds of Formula I to inhibit growth of tumor cells in vitro was determined as follows.

Stock cultures of the DU 145 prostate carcinoma cell line were maintained in RPMI medium 1640 containing 10% (v/v) fetal bovine serum, 2 mM L-glutamine, 1 mM sodium pyruvate, 50 units/ml penicillin, and 50 μg/ml streptomycin at 37° C. in 5% CO₂ humidified atmosphere. Cells were cultured in 75-cm² culture flasks and subcultures were established every 3 to 4 days so as not to allow the cells to exceed 90% confluence.

DU 145 cells were harvested for proliferation assays by trypsinization (0.05% trypsin/0.53 mM EDTA), washed twice in culture medium, resuspended in appropriate volume of medium, and then counted using a hemacytometer. Cells were seeded in wells of flat-bottom 96-well plates at a density of 5,000 cell/well in 100 μl. Cells were allowed to attach for 1.5 to 2 hours at 37° C.

Compounds were diluted from 10 mM stock solutions in DMSO. Serial 3-fold dilutions were performed in medium containing 0.6% DMSO in wells (in triplicate) of a 96-well U-bottom plates starting with a 60 μM solution. After dilutions were completed, 100 μl of each compound dilution (in triplicate) was transferred to designated triplicate wells of the 96-well plate containing cells in 100 μl of medium. Final concentrations of the dose-response for compounds in assay plates ranged from 0.12 to 30 μM. Control wells (cells with notreatment) received 100 μl of 0.6% DMSO in culture medium. Wells containing medium with no cells served as the background wells. Cells were cultured with the compounds for 48 and 72 hours at 37° C. in a humidified CO₂ incubator.

Cell proliferation was assessed by measuring fluorescence after the addition of the fluorogenic redox indicator, Alamar Blue™ (BioSource International). Ten μl of Alamar Blue™ was added to each well of the 96-well plate(s) 3 to 4 hours prior to the end of the incubation period. Assay plates were read in a fluorescence plate reader (excitation, 530 nM; emission, 620 nM). G₁₅₀ values (concentration at which the growth of the tumor cells was inbibited by 50%) for compounds were determined by plotting the percent control fluorescence against the logarithm of the compound concentration. The compounds of this invention inhibited the growth of the tumor cells.

### Pharmaceutical Composition Examples

The following are representative pharmaceutical formulations containing a compound of Formula I

### Tablet Formulation

The following ingredients are mixed intimately and pressed into single scored tablets.

| | Quantity per |
|---|---|
| Ingredient | tablet, mg |
| compound of this invention | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| | Quantity per |
|---|---|
| Ingredient | capsule, mg |
| compound of this invention | 200 |
| lactose, spray-dried | 148 |
| magnesium stearate | 2 |

### Suspension Formulation

The following ingredients are mixed to form a suspension for oral administration.

| Ingredient | Amount |
|---|---|
| compound of this invention | 1.0 g |
| fumaric acid | 0.5 g |
| sodium chloride | 2.0 g |
| methyl paraben | 0.15 g |
| propyl paraben | 0.05 g |
| granulated sugar | 25.5 g |
| sorbitol (70% solution) | 12.85 g |
| Veegum K (Vanderbilt Co.) | 1.0 g |
| flavoring | 0.035 ml |
| colorings | 0.5 mg |
| distilled water | q.s. to 100 ml |

### Injectable Formulation

The following ingredients are mixed to form an injectable formulation.

| Ingredient | Amount |
|---|---|
| compound of this invention | 1.2 g |
| sodium acetate buffer solution, | 0.4 M 2.0 ml |
| HCl (1 N) or NaOH (1 N) | q.s. to suitable pH |
| water (distilled, sterile) | q.s. to 20 ml |

All of the above ingredients, except water, are combined and heated to 60-70.degree. C. with stirring. A sufficient quantity of water at 60.degree. C. is then added with vigorous stirring to emulsify the ingredients, and water then added q.s. to 100 g.

### Suppository Formulation

A suppository of total weight 2.5 g is prepared by mixing the compound of the invention with Witepsol.RTM. H-15 (triglycerides of saturated vegetable fatty acid; Riches-Nelson, Inc., New York), and has the following composition:

| | |
|---|---|
| compound of the invention | 500 mg |
| Witepsol ™ H-15 | balance |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled. All patents, patent applications and publications cited in this application are hereby incorporated by reference in their entirety for all purposes to the same extent as if each individual patent, patent application or publication were so individually denoted.

We claim:

1. A compound of Formula I: 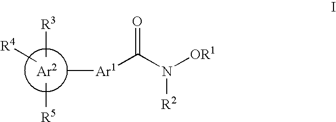
wherein:
R¹ is hydrogen or alkyl;
R² is hydrogen;
Ar¹ is phenylene or a six membered heteroarylene ring containing one or two nitrogen ring atoms, the rest of the ring atoms being carbon; wherein said Ar¹ group is optionally substituted with one or two groups independently selected from alkyl, halo, hydroxy, alkoxy, haloalkoxy, or haloalkyl;
Ar² is aryl, benzimidazol-2-yl, cycloalkyl or heterocycloalkyl;
R³ is hydrogen, alkyl, halo, hydroxy, or alkoxy; and
R⁴ and R⁵ are independently selected from the group consisting of hydrogen, alkyl, halo, haloalkyl, nitro, cyano, carboxy, carboxyalkyl, alkoxycarbonyl, optionally substituted phenyl, optionally substituted heteroaryl, optionally substituted heterocycloalkyl, cycloalkyl, heterocycloaminoalkyl, —X—R⁶, or —(C₁₋₆alkylene)-Y—R⁷ where X and Y are independently —O—, —S—, —SO—, —SO₂—, —NR⁸—, —CO—, —NR⁹CO—, —CONR¹⁰—, —NR¹¹SO₂—, —SO₂NR¹²—, —NHC(O)O—, —OC(O)NH—, —NR¹³CONR¹⁴—, or —NR¹⁵SO₂NR¹⁶— where R⁶ and R⁷ are independently hydrogen, alkyl, hydroxyalkyl, optionally substituted phenyl, optionally substituted heteroaryl, optionally substituted heterocycloalkyl, cycloalkyl, optionally substituted phenylalkyl, optionally substituted phenoxyalkyl, optionally substituted phenylalkenyl, optionally substituted phenylaminoalkyl, optionally substituted heteroaralkyl, optionally substituted heteroaryloxyalkyl, optionally substituted heterocycloalkylalkyl, or cycloalkylalkyl, R⁸, R⁹, R¹¹, R¹³, and R¹⁵ are independently hydrogen, alkyl, hydroxyalkyl, alkoxyalkyl, or optionally substituted phenylalkyl; R¹⁰, R¹², R¹⁴, and R¹⁶ are independently hydrogen, alkyl, optionally substituted phenylalkyl, alkoxy, hydroxyalkyl, haloalkyl, alkoxyalkyl, carboxyalkyl, cyanoalkyl, aminoalkyl, aminocarbonylalkyl, alkylaminocarbonylalkyl, dialkylaminocarbonylalkyl, or acyl or R⁴ and R⁵ together form methylenedioxy; and individual isomers, mixtures of isomers; or a pharmaceutically acceptable salt thereof provided that: (i) at least one of R³, R⁴ and R⁵ is not hydrogen; (ii) when Ar² is cycloalkyl, then at least two of R³, R⁴ and R⁵ are hydrogen; (iii) when R¹ and R³ are hydrogen, Ar¹ is phenylene and Ar² is phenyl, and one of R⁴ and R⁵ is methoxy, then the other of R⁴ and R⁵ is not —OR⁶ where R⁶ is cyclopentyl or phenylpentyl; (iv) when Ar¹ is phenylene and Ar² is phenyl then at least one of R³, R⁴ and R⁵ is not alkyl; (v) when Ar¹ is phenylene, Ar² is aryl and is located at the 3-position of the phenylene ring, then Ar² is not substituted with an optionally substituted phenyl; (vi) when Ar¹ is phenylene and Ar² is phenyl, and R⁴ or R⁵ is —CONR¹⁰R⁶ or —(C₁₋₆alkylene)-CONR¹⁰R⁷ then said R⁴ or R⁵ is not located at the 4-position of the phenyl ring; and (vii) when Ar¹ is phenylene and Ar² is phenyl and two of R³, R⁴ and R⁵ are hydrogen, then the remaining of R³, R⁴ and R⁵ is not nitro.

2. The compound of claim 1 wherein said compound is represented by Formula Ib: 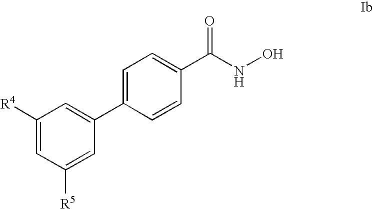
wherein:
R⁴ is hydrogen, alkyl, halo, haloalkyl, cyano, carboxy, carboxyalkyl, alkoxycarbonyl, heterocycloaminoalkyl, —X—R⁶, or —(C₁₋₆alkylene)-Y—R⁷ where X and Y are independently —O—, —S—, —SO—, —SO₂—, —NR⁸—, —CO—, —NR⁹CO—, —CONR¹⁰—, —NR¹¹SO₂—, —SO₂NR¹², —NHC(O)O—, —OC(O)NH—, —NR¹³CONR¹⁴—, or —NR¹⁵SO₂NR¹⁶—;
R⁵ is —X—R⁶ or —(C₁₋₆alkylene)-Y—R⁷ where X and Y are independently —O—, —S—, —SO—, —SO₂—, —NR⁸—, —CO—, —NR⁹CO—, —CONR¹⁰—, —NR¹¹SO₂—, —SO₂NR¹²—, —NHC(O)O—, —OC(O)NH—, —NR¹³CONR¹⁴—, or —NR¹⁵SO₂NR¹⁶— where:
each R⁶ and R⁷ is independently hydrogen, alkyl, hydroxyalkyl, optionally substituted phenyl, optionally substituted heteroaryl, optionally substituted heterocycloalkyl, cycloalkyl, optionally substituted phenylalkyl, optionally substituted phenyalkenyl, optionally substituted phenoxyalkyl, optionally substituted phenylaminoalkyl, optionally substituted heteroaralkyl, optionally substituted heteroaryloxyalkyl, optionally substituted heterocycloalkylalkyl, or cycloalkylalkyl,
each R⁸, R⁹, R¹¹, R¹³, and R¹⁵ is independently hydrogen, alkyl, hydroxyalkyl, alkoxyalkyl, or optionally substituted phenylalkyl; and
each R¹⁰, R¹², R¹⁴, and R¹⁶ is independently hydrogen, alkyl, optionally substituted phenylalkyl, alkoxy, hydroxyalkyl, haloalkyl, alkoxyalkyl, carboxyalkyl, cyanoalkyl, aminoalkyl, aminocarbonylalkyl, alkylaminocarbonylalkyl, dialkylaminocarbonylalkyl, or acyl;
and individual stereoisomers, mixtures of stereoisomers; or a pharmaceutically acceptable salt thereof provided that (i) when one of R⁴ and R⁵ is methoxy, then the other of R⁴ and R⁵ is not —OR⁶ where R⁶ is cyclopentyl or phenylpentyl.

3. The compound of claim 1 wherein said compound is represented by Formula Ib: 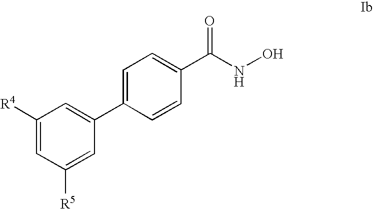
wherein:
R⁴ is hydrogen, alkyl, halo, haloalkyl, cyano, carboxy, carboxyalkyl, alkoxycarbonyl, heterocycloaminoalkyl, —X—R⁶, or —(C₁₋₆alkylene)-Y—R⁷ where X and Y are independently —O—, —S—, —SO—, —SO₂—, —NR⁸—, —CO—, —NR⁹CO—, —CONR¹⁰—, —NR¹¹SO₂—, —SO₂NR¹²—, —NHC(O)O—, —OC(O)NH—, —NR¹³CONR¹⁴—, or —NR¹⁵SO₂NR¹⁶—;
R⁵ is —X—R⁶ or —(C₁₋₆alkylene)-Y—R⁷ where X and Y are independently —O—, —S—, —SO—, —SO₂—, —NR⁸—, —CO—, —NR⁹CO—, —CONR¹⁰—, —NR¹¹SO₂—, —SO₂NR¹²—, —NHC(O)O—, —OC(O)NH—, —NR¹³CONR¹⁴—, or —NR¹⁵SO₂NR¹⁶— where:
each R⁶ and R⁷ is independently hydrogen, alkyl, hydroxyalkyl, optionally substituted phenyl, optionally substituted heteroaryl, optionally substituted heterocycloalkyl, cycloalkyl, optionally substituted phenylalkyl, optionally substituted phenyalkenyl, optionally substituted phenoxyalkyl, optionally substituted phenylaminoalkyl, optionally substituted heteroaralkyl, optionally substituted heteroaryloxyalkyl, optionally substituted heterocycloalkylalkyl, or cycloalkylalkyl,
each R⁸, R⁹, R¹¹, R¹³, and R¹⁵ is independently hydrogen, alkyl, hydroxyalkyl, alkoxyalkyl, or optionally substituted phenylalkyl; and
each R¹⁰, R¹², R¹⁴, and R¹⁶ is independently hydrogen, alkyl, optionally substituted phenylalkyl, alkoxy, hydroxyalkyl, haloalkyl, alkoxyalkyl, carboxyalkyl, cyanoalkyl, aminoalkyl, aminocarbonylalkyl, alkylaminocarbonylalkyl, dialkylaminocarbonylalkyl, or acyl;
and individual stereoisomers, mixtures of stereoisomers; or a pharmaceutically acceptable salt thereof provided that (i) when one of R⁴ and R⁵ is methoxy, then the other of R⁴ and R⁵ is not —OR⁶ where R⁶ is cyclopentyl or phenylpentyl, (ii) when R⁴ is hydrogen, alkyl, halo, haloalkyl, cyano, carboxy, alkoxycarbonyl, or —X—R⁶ [where X is —O—, —S—, —SO—, —NR⁸—, or —CO— where R⁶ and R⁸ are independently hydrogen or alkyl), then R⁵ is not —X—R⁶ [where X is —O—, —S—, —SO—, or —NR⁸— where R⁶ and R⁸ are independently hydrogen or alkyl]; (iii) when R⁴ and/or R⁵ are —X—R⁶ [where X is —CONR¹⁰—, —SO₂NR¹²—, —NR¹³CONR¹⁴—, or —NR¹⁵SO₂NR¹⁶—, then both R⁶ and R¹⁰, R¹², R¹⁴, and R¹⁶ are not simultaneously hydrogen; and (iv) when R⁴ and/or R⁵ are —X—R⁶ where X is —NR⁹CO—, —NR¹¹SO₂—, —NHC(O)O—, or —OC(O)NH—, then R⁶ is not hydrogen.

4. The compound of claim 3 wherein said compound is represented by Formula Ic: 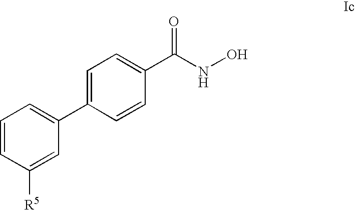
wherein:
R⁵ is —X—R⁶ or —(C₁₋₆alkylene)-Y—R⁷ where:
X is —NR⁸—, —NR⁹CO—, —NR¹¹SO₂—, —NR¹³CONR¹⁴—, or —NR¹⁵SO₂NR¹⁶—;
Y is —O—, —S—, —SO—, —SO₂—, —NR⁸—, —CO—, —NR⁹CO—, or —CONR¹⁰—;
R⁶ is alkyl, optionally substituted phenyl, optionally substituted heteroaryl, optionally substituted phenylalkyl, optionally substituted phenoxyalkyl, optionally substituted heteroaralkyl, or optionally substituted phenylalkenyl;
R⁷ is hydrogen, alkyl, hydroxyalkyl, optionally substituted phenyl, optionally substituted heteroaryl, optionally substituted heterocycloalkyl, cycloalkyl, optionally substituted phenylalkyl, optionally substituted phenoxyalkyl, optionally substituted heteroaralkyl, optionally substituted heteroaryloxyalkyl, optionally substituted heterocycloalkylalkyl, or cycloalkylalkyl;
R⁸, R⁹, R¹¹, R¹³, and R¹⁵ are independently hydrogen, alkyl, hydroxyalkyl, alkoxyalkyl, or optionally substituted phenylalkyl; and
R¹⁰, R¹⁴, and R¹⁶ are independently hydrogen, alkyl, optionally substituted phenylalkyl, alkoxy, hydroxyalkyl, or alkoxyalkyl; or
a pharmaceutically acceptable salt thereof provided that when R⁵ is —NR⁶R⁸ then R⁶ is not alkyl.

5. The compound of claim 4 wherein X is —NR⁸—, —NR⁹CO—, —NR¹¹SO₂—, or —NR¹³CONR¹⁴—; Y is —O—, —NR⁸—, —CO—, —NR⁹CO—, or —CONR¹⁰—; R⁶ is alkyl, optionally substituted phenyl, optionally substituted heteroaryl, optionally substituted phenylalkyl, optionally substituted phenoxyalkyl, or optionally substituted heteroaralkyl; R⁷ is alkyl, optionally substituted phenyl, optionally substituted heterocycloalkyl, optionally substituted phenylalkyl, optionally substituted heteroaryl, or optionally substituted heteroaralkyl; each R⁸, R⁹, R¹¹, and R¹³ are independently hydrogen, alkyl, hydroxyalkyl, or alkoxyalkyl; and R¹⁰ and R¹⁴ are independently hydrogen or alkyl.

6. The compound of claim 4 wherein R⁵ is methylsulfonylamino, phenylsulfonylamino, phenylureido, 3-chlorophenylsulfonylamino, 4-fluorophenylsulfonylamino, 3,4-dichloro-phenylsulfonylamino, phenylcarbonylamino, benzylsulfonylamino, 4-chlorophenyl-sulfonylamino, 3-trifluoromethylphenylsulfonylamino, 4-methoxyphenylsulfonylamino, 3,4-dichlorophenylcarbonylamino, 3-methoxyphenylureido, 3,4-dimethoxyphenylcarbonylamino, 2,5-dimethoxyphenylsulfonylamino, 4-trifluoromethoxyphenylsulfonylamino, 3-fluorophenyl-carbonylamino, 2,4-dichlorophenylcarbonylamino, 4-methylphenylcarbonylamino, 3-trifluoromethylphenylcarbonylamino, 3,4-methylenedioxyphenylcarbonylamino, 4-methoxy-phenylcarbonylamino, 2-propylcarbonylamino, benzylcarbonylamino, 2-phenylethylcarbonyl-amino, 2-(4-trifluoromethylphenyl)ethylcarbonylamino, 2-(4-methoxyphenyl)ethylcarbonyl-amino, 3,5-dichlorophenylcarbonylamino, phenoxymethylcarbonylamino, 3-methylbutyl-carbonylamino, thiophen-2-ylmethylcarbonylamino, 2-(2,4-dichlorophenyl)ethylcarbonyl-amino, 2-(3,4-methylenedioxyphenyl)ethylcarbonylamino, 4-trifluoromethylphenylcarbonyl-amino, 4-ethoxyphenylcarbonylamino, 4-dimethylaminophenylcarbonylamino, 4-fluorophenyl-carbonylamino, 2,4-difluorophenylcarbonylamino, 4-chlorophenylcarbonylamino, 4-isopropyl-phenylcarbonylamino, 4-trifluoromethoxyphenylcarbonylamino, 3-fluoro-4-methoxy-phenylcarbonylamino, 4-methoxy-2-methylphenylcarbonylamino, 2,4-dimethoxyphenyl-carbonylamino, 4-chloro-2-methoxyphenylcarbonylamino, pyridin-4-ylcarbonylamino, pyridin-3-ylcarbonylamino, 2-methylphenylcarbonylamino, 2,4-dimethylphenyl-carbonylamino, 2,5-dimethylphenylcarbonylamino, 2-methylthiophen-5-ylcarbonylamino, benzylamino, 4-methoxybenzylamino, 4-methylbenzylamino, 2-(4-methoxyphenyl)-ethylaminocarbonylmethyl, 4-chlorobenzylaminocarbonylmethyl, dimethylaminocarbonylmethyl, morpholin-4-ylcarbonylmethyl, N-benzyl-N-methylamino-carbonylmethyl, 2-(phenyl)ethylaminocarbonylmethyl, 4-chlorophenylaminocarbonylmethyl, phenylcarbonylaminomethyl, pyridin-3-ylmethylcarbonylaminomethyl, N-benzoyl-N-(2-hydroxyethyl)aminomethyl, N-benzyl-N-(2-hydroxyethyl)aminomethyl, N-benzyl-N-(2-methoxyethyl)aminomethyl, benzylaminomethyl, 2-indol-3-ylethylaminomethyl, 3,4-methylenedioxyphenylmethylaminomethyl, pyridin-4-ylmethylaminomethyl, pyridin-3-yloxy-methyl, 2-pyridin-3-ylethylaminomethyl, phenoxymethyl, 4-methylphenoxymethyl, 4-chlorophenoxymethyl, 3-phenylpropylaminomethyl, phenylaminomrethyl, 4-methylphenylaminomethyl, or 4-chlorophenylaminomethyl.

7. The compound of claim 3 wherein said compound is represented by Formula Id: 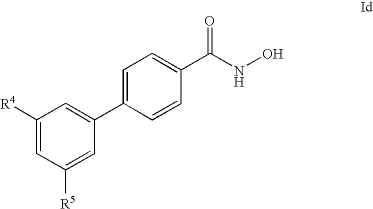
wherein:
R⁴ is carboxy, heterocycloaminoalkyl, —CO-(optionally substituted heterocycloalkyl), —CONR⁶R¹⁰, or —(C₁₋₆alkylene)-Y—R⁷ where Y is —NR⁸— or —O—; R⁶ is hydrogen, alkyl, hydroxyalkyl, optionally substituted phenyl, optionally substituted heteroaryl, optionally substituted phenylalkyl, optionally substituted phenoxyalkyl, optionally substituted heteroaralkyl, or optionally substituted phenylalkenyl; R⁷ is alkyl, optionally substituted phenyl, optionally substituted phenylalkyl, or optionally substituted phenylalkenyl; R⁸ is alkyl, optionally substituted phenylalkyl, hydroxyalkyl, or alkoxyalkyl; and R¹⁰ is hydrogen, alkyl, optionally substituted phenylalkyl, alkoxy, hydroxyalkyl, alkoxyalkyl, carboxyalkyl, cyanoalkyl, aminoalkyl, aminocarbonylalkyl, alkylaminocarbonylalkyl, dialkylaminocarbonylalkyl, or acyl; and
R⁵ is —X—R⁶ or —(C₁₋₆alkylene)-Y—R⁷ where X is —NR⁸—, —NR⁹CO—, —NR¹¹SO₂—, —NR¹³CONR¹⁴—, or —NR¹⁵SO₂NR¹⁶—; Y is —O—, —S—, —SO—, —SO₂—, —NR⁸—, —CO—, —NR⁹CO—, or —CONR¹⁰—; R⁶ is alkyl, optionally substituted phenyl, optionally substituted heteroaryl, optionally substituted phenylatkyl, optionally substituted phenoxyalkyl, optionally substituted phenylaminoalkyl, optionally substituted heteroaralkyl, or optionally substituted phenylalkenyl; R⁷ is alkyl, optionally substituted phenyl, optionally substituted phenylalkyl, optionally substituted phenylalkenyl, or optionally substituted heterocycloalkyl; R⁸, R⁹, R¹¹, R¹³, and R¹⁵ are independently hydrogen, alkyl, or optionally substituted phenylalkyl; and R¹⁰, and R¹⁶ are independently hydrogen, alkyl, optionally substituted phenylalkyl, alkoxy, hydroxyalkyl, or alkoxyalkyl;
or a pharmaceutically acceptable salt thereof provided both R⁶ and R¹⁰ are not simultaneously hydrogen.

8. The compound of claim 7 wherein:
R⁴ is carboxy, heterocycloaminoalkyl, —CO-(optionally substituted heterocycloamino), —CONR⁶R¹⁰, or —(C₁₋₆alkylene)-Y—R⁷ where Y is —NR⁸— or —O—; R⁶ is hydrogen, alkyl, hydroxyalkyl, optionally substituted phenyl, or optionally substituted phenylalkyl; R⁷ is alkyl, optionally substituted phenyl, optionally substituted phenylalkyl, or optionally substituted phenylalkenyl; R⁸ is alkyl; and R¹⁰ is hydrogen, alkyl, alkoxy, hydroxyalkyl, alkoxyalkyl, carboxyalkyl, aminoalkyl, aminocarbonylalkyl, alkylaminocarbonylalkyl, or dialkylaminocarbonylalkyl; and
R⁵ is —X—R⁶ where X is —NHCO—, —NHSO₂—, or —NHCONH— and R⁶ is alkyl, optionally substituted phenyl, optionally substituted heteroaryl, optionally substituted phenylalkyl, optionally substituted phenylaminoalkyl, optionally substituted heteroaralkyl, or optionally substituted phenylalkenyl.

9. The compound of claim 7 wherein:
R⁴ is —CONR⁶R¹⁰ where R⁶ is hydrogen, alkyl, hydroxyalkyl, optionally substituted phenyl, or optionally substituted phenylalkyl; and R¹⁰ is hydrogen, alkyl, alkoxy, hydroxyalkyl, alkoxyalkyl, carboxyalkyl, aminoalkyl, aminocarbonylalkyl, alkylaminocarbonylalkyl, or dialkylaminocarbonylalkyl provided both R⁶ and R¹⁰ are not hydrogen; and
R⁵ is —NHCOR⁶ where R⁶ is optionally substituted phenyl, optionally substituted heteroaryl, optionally substituted phenylalkyl, optionally substituted phenylaminoalkyl, optionally substituted heteroaralkyl, or optionally substituted phenylalkenyl.

10. The compound of claim 7 wherein:
R⁴ is —CO-(optionally substituted heterocycloamino); and
R⁵ is —NHCOR⁶ where R⁶ is optionally substituted phenyl, optionally substituted heteroaryl, optionally substituted phenylalkyl, optionally substituted phenylaminoalkyl, optionally substituted heteroaralkyl, or optionally substituted phenylalkenyl.

11. A compound selected from the group consisting of:
N-hydroxy-4-(3-methoxyphenyl)benzamide;
N-hydroxy-4-(2-methoxyphenyl)benzamide;
N-hydroxy-4-(2,4-dichlorophenyl)benzamide;
N-hydroxy-4-(3,5-dichlorophenyl)benzamide;
N-hydroxy-4-(5-chloro-2-methoxyphenyl)benzamide;
N-hydroxy-4-(3-isopropylphenyl)benzamide;
N-hydroxy-4-(3-nitrophenyl)benzamide;
N-hydroxy-4-(3-methylsulfonylaminophenyl)benzamide;
N-hydroxy-4-(3-aminophenyl)benzamide;
N-hydroxy-4-(3-chlorophenyl)benzamide;
N-hydroxy-4-(3-trifluoromethylphenyl)benzamide;
N-hydroxy-4-(2-trifluoromethylphenyl)benzamide;
N-hydroxy-4-(2,4-difluorophenyl)benzamide;
N-hydroxy-4-(2-fluorophenyl)benzamide;
N-hydroxy-4-(2,4-dimethoxyphenyl)benzamide;
N-hydroxy-4-(3,4-methylenedioxyphenyl)benzamide;
N-hydroxy-4-(3-methylsulfonylphenyl)benzamide;
N-hydroxy-4-(2,3-dichlorophenyl)benzamide;
N-hydroxy-4-(4-thiophen-3-ylphenyl)benzamide;
N-hydroxy-4-(3-cyanophenyl)benzamide;
N-hydroxy-4-(3-phenylsulfonylaminophenyl)benzanfide;
N-hydroxy-4-(2-methylphenyl)benzamide;
N-hydroxy-4-(2,3-difluorophenyl)benzamide;
N-hydroxy-4-(3,5-dimethylphenyl)benzamide;
N-hydroxy-4-(3-hydroxyphenyl)benzamide;
N-hydroxy-4-(3-hydroxymethylphenyl)benzamide;
N-hydroxy-4-(2,6-difluorophenyl)benzamide;
N-hydroxy-4-(2,5-dimethylphenyl)benzamide;
N-hydroxy-4-(2,3-dimethylphenyl)benzamide;
N-hydroxy-4-(2-chlorophenyl)benzamide;
N-hydroxy-4-(4-phenylsulfonylaminophenyl)benzamide;
N-hydroxy-4-[3-(3-chlorophenylsulfonylamino)phenyl]benzamide;
N-hydroxy-4-[3-(4-fluorophenylsulfonylamino)phenyl]benzamide;
N-hydroxy-4-[3-(3,4-dichlorophenylsulfonylamino)phenyl]benzamide;
N-hydroxy-4-[3-(phenylcarbonylamino)phenyl]benzamide;
N-hydroxy-4-[3-(benzylsulfonylamino)phenyl]benzamide;
N-hydroxy-4-[3-(4-chlorophenylsulfonylamino)phenyl]benzamide;
N-hydroxy-4-[3-(3-trifluoromethylphenylsulfonylamino)phenyl]benzamide;
N-hydroxy-4-(2,4-diphenylbenzimidazol-2-yl)benzamide;
N-hydroxy-4-(benzimidazol-2-yl)benzamide;
N-hydroxy-4-[3-(4-methoxyphenylsulfonylamino)phenyl]benzamide;
N-hydroxy-4-[3-(3,4-dichlorophenylcarbonylamino)phenyl]benzamide;
N-hydroxy-4-(1-methylbenzimidazol-2-yl)benzamide;
N-hydroxy-4-[3-(3-methoxyphenylureido)phenyl]benzamide;
N-hydroxy-4-[3-(3,4-dimethoxyphenylcarbonylamino)phenyl]benzamide;
N-hydroxy-4-[3-(2,5-dimethoxyphenylsulfonylamino)phenyl]benzamide;
N-hydroxy-4-[3-(4-trifluoromethoxyphenylsulfonylamino)phenyl]benzamide;
N-hydroxy-4-[3-(3-fluorophenylcarbonylamino)phenyl]benzamide;
N-hydroxy-4-[3-(2,4-dichlorophenylcarbonylamino)phenyl]benzamide;
N-hydroxy-4-[3-(phenylcarbonylamino)phenyl]benzamide;
N-hydroxy-4-[3-(4-methylphenylcarbonylamino)phenyl]benzamide;
N-hydroxy-4-[3-(3-trifluoromethylphenylcarbonylamino)phenyl]benzamide;
N-hydroxy-4-[5-(phenylaminocarbonyl)-3-(phenylcarbonylamino)phenyl]benzamide;
N-hydroxy-4-[5-(phenylaminocarbonyl)-3-(phenylsulfonylamino)phenyl]benzamide;
N-hydroxy-4-(3-benzyloxyphenyl)benzamide;
N-hydroxy-4-[3-(3-methoxybenzyloxy)phenyl]benzamide;
N-hydroxy-4-[3-(3,4-methylenedioxyphenylcarbonylamino)phenyl]benzamide;
N-hydroxy-4-[3-(N-3-methylbutylaminocarbonyl)phenyl]benzamide;
N-hydroxy-4-[3-(N-methylaminocarbonyl)phenyl]benzamide;
N-hydroxy-4-[3-(2-propylcarbonylamino)phenyl]benzamide;
N-hydroxy-4-(5-fluoro-7-phenoxybenzimidazol-2-yl)benzamide;
N-hydroxy-4-[5-fluoro-7-(2-phenylethoxy)benzimidazol-2-yl]benzamide;
N-hydroxy-4-[5-fluoro-7-(2-methoxyphenoxy)benzimidazol-2-yl]benzamide;
N-hydroxy-4-[3-(benzylcarbonylamino)phenyl]benzamide;
N-hydroxy-4-[3-(2-phenylethylcarbonylawino)phenyl]benzamide;
N-hydroxy-4-[5-fluoro-7-(tetydroyfuran-2-ylmethoxyoxy)benzimidazol-2-yl]benzamde;
N-hydroxy-4-{3-[2-(4-trifluorophenyl)ethylcarbonylamino]phenyl}benzamide;
N-hydroxy-4-{3-[2-(4-methoxyphenyl)ethylcarbonylamino]phenyl}benzamide;
N-hydroxy-4-[5-(N-benzylaminocarbonyt)-3-(phenylcarbonylamino)phenyl]benzamide;
N-hydroxy-4-[5-carboxy-3-(phenylcarbonylamino)phenyl]benzamide;
N-hydroxy-4-[5-(N,N-dimethylaminocarbonyl)-3-(phenylcarbonylamino)phenyl]benzamide;
N-hydroxy-4-[5-(morpholin-4-ylcarbonyl)-3-(phenylcarbonylamino)phenyl]benzamide;
N-hydroxy-4-[5-(N,N-dimethylaminocarbonyl)-3-(4-methylphenylcarbonylamino)phenyl]-benzamide;
N-hydroxy-4-[3-(4-methylphenylcarbonylamino)-5-(N-phenylaminocarbonyl)phenyl]-benzamide;
N-hydroxy-4-[3-(3,5-dichlorophenylcarbonylamino)phenyl]benzamide;
N-hydroxy-4-[3-(phenoxymethylcarbonylamino)phenyl]benzamide;
N-hydroxy-4-[5-fluoro-7-(2-methylpropoxy)benzimidazol-2-yl]benzamide;
N-hydroxy-4-[5-fluoro-7-(cyclohexyloxy)benzimidazol-2-yl]benzamide;
N-hydroxy-4-[3-(3-methylbutylcarbonylamino)phenyl]benzamide;
N-hydroxy-4-[3-(thiophen-2-yinethylcarbonylamino)phenyl]benzamide;
N-hydroxy-4-{3-[2-(2,4-dichlorophenyl)ethylcarbonylamino]phenyl}benzamide;
N-hydroxy-4-{3-[2-(3,4-methylenedioxyphenyl)ethylcarbonylamino]phenyl}benzamide;
N-hydroxy-4-[5-(N,N-dimethylaminocarbonyl)-3-(4-methoxyphenylcarbonylamino)phenyl]-benzamide;
N-hydroxy-4-[3-(4-methoxyphenylcarbonylamino)-5-(morpholin-4-ylcarbonyl)phenyl]-benzamide;
N-hydroxy-4-[3-(4-methoxyphenylcarbonylamino)-5-(phenylaminocarbonyl)phenyl]-benzamide;
N-hydroxy-4-[5-(N,N-dimethylaminocarbonyl)-3-(3,4-dimethoxyphenylcarbonylamino)-phenyl]-benzamide;
N-hydroxy-4-[3-(3,4-dimethoxyphenylcarbonylamino)-5-(morpholin-4-ylcarbonyl)phenyl]-benzamide;
N-hydroxy-4-[3-(3,4-dimethoxyphenylcarbonylamino)-5-(phenylaminocarbonyl)phenyl]-benzamide;
N-hydroxy-4-[3-(4-trifluorophenylcarbonylamino)phenyl]benzamide;
N-hydroxy-4-[3-(4-ethoxyphenylcarbonylamino)phenyl]benzamide;
N-hydroxy-4-[3-(4-N,N-dimethylaminophenylcarbonylamino)phenyl]benzamide;
N-hydroxy-4-[3-(4-fluorophenylcarbonylamino)phenyl]benzamide;
N-hydroxy-4-[3-(2,4-difluorophenylcarbonylamino)phenyl]benzamide;
N-hydroxy-4-[3-(4-chlorophenylcarbonylamino)phenyl]benzamide;
N-hydroxy-4-[3-(N,N-dimethylaminocarbonyl)phenyl]benzamide;
N-hydroxy-4-[3-(4-methylphenylcarbonylamino)-5-(piperidin-1-ylcarbonyl)phenyl]-benzamide;
N-hydroxy-4-[3-(4-methylphenylcarbonylamino)-5-(piperazin-1-ylcarbonyl)phenyl]-benzamide;
N-hydroxy-4-[5-(N-methylaminocarbonyl)-3-(4-methylphenylcarbonylamino)phenyl]-benzamide;
N-hydroxy-4-[5-(N-carboxymethyl-N-methylaminocarbonyl)-3-(4-methylphenylcarbonyl-amino)phenyl]benzamide;
N-hydroxy-4-[5-(N-aminocarbonylmethylaminocarbonyl)-3-(4-methylphenylcarbonyl-amino)phenyl]-benzamide;
N-hydroxy-4-{5-[N-(2-N-methylaminoethyl)-N-(methyl)aminocarbonyl]-3-(4-methylphenyl-carbonylamino)phenyl}-benzamide;
N-hydroxy-4-[5-(N-carboxymethylaminocarbonyl)-3-(4-methylphenylcarbonylamino)-phenyl]-benzamide;
N-hydroxy-4-[3-(4-isopropylphenylcarbonylamino)phenyl]benzamide;
N-hydroxy-4-[3-(4-trifluoromethoxyphenylcarbonylamino)phenyl]benzamide;
N-hydroxy-4-[3-(3-fluoro-4-methoxyphenylcarbonylamino)phenyl]benzamide;
N-hydroxy-4-[3-(4-methoxy-2-methylphenylcarbonylamino)phenyl]benzamide;
N-hydroxy-4-[3-(2,4-dimethoxyphenylcarbonylamino)phenyl]benzamide;
N-hydroxy-4-[3-(4-chloro-2-methoxyphenylcarbonylamino)phenyl]benzamide;
N-hydroxy-4-[3-(pyridin-4-ylcarbonylamino)phenyl]benzamide;
N-hydroxy-4-[3-(pyridin-3-ylcarbonylamino)phenyl]benzamide;
N-hydroxy-4-[3-(morpholin-4-ylcarbonylamino)phenyl]benzamide;
N-hydroxy-4-[3-(N-methyl-N-phenylaminocarbonyl)phenyl]benzamide;
N-hydroxy-4-[3-(4-chlorophenylaminocarbonyl)phenyl]benzamide;
N-hydroxy-4-{3-[N-2-(4-methoxyphenyl)ethylaminocarbonyl]phenyl}benzamide;
N-hydroxy-4-[5-(N,N-dimethylaminocarbonyl)-3-(3,4-methylenedioxyphenylcarbonyl-amino)phenyl]benzamide;
N-hydroxy-4-[3-(3,4-methylcnedioxyphenylcarbonylamino)-5-(morpholin-4-ylcarbonyl)-phenyl]benzamide;
N-hydroxy-4-[3-(3,4-methylenedioxyphenylcarbonylamino)-5-(N-phenylaminocarbonyl)-phenyl]benzamide;
N-hydroxy-4-[3-(2,4-dichlorophenylcarbonylamino)-5-(NN-dimethylaminocarbonyl)-phenyl]benzamide;
N-hydroxy-4-[3-(2,4-dichlorophenylcarbonylamino)-5-(N-phenylaminocarbonyl)phenyl]-benzamide;
N-hydroxy-4-[5-(N,N-dimethylaminocarbonyl)-3-(4-chlorophenylcarbonylamino)-phenyl]benzamide;
N-hydroxy-4-[5-(morpholin-4-ylcarbonyl)-3-(4-chlorophenylcarbonylamino)-phenyl]benzamide;
N-hydroxy-4-[3-(N-4-chlorobenzylaminocarbonyl)phenyl]benzamide;
N-hydroxy-4-[3-(N-2-phenylethylaminocarbonyl)phenyl]benzamide;
N-hydroxy-4-[3-(N-2-hydroxyethylaminocarbonyl)phenyl]benzamide;
N-hydroxy-4-[3-(piperidin-1-ylcarbonyl)phenyl]benzamide;
N-hydroxy-4-[5-(N-methyl-N-ethylaminocarbonyl)-3-(4-methylphenylcarbonylamino)-phenyl]benzamide;
N-hydroxy-4-[3-(4-methylphenylcarbonylamino)-5-(4-methylpiperazin-1-ylcarbonyl)-phenyl]-benzamide;
N-hydroxy-4-[5-(3-(RS)-aminocarbonylpiperidin-1-ylcarbonyl)-3-(4-methylphenylcarbonyl-amino) phenyl)benzamide;
N-hydroxy-4-[5-(N,N-diethylaminocarbonyl)-3-(4-methylphenylcarbonylamino)-phenyl]benzamide;
N-hydroxy-4-{5-[N-2-(N-methylamino)ethylaminocarbonyl]-3-(4-methylphenylcarbonyl-amino)-phenyl}benzamide;
N-hydroxy-4-[5-(4-RS-hydroxypiperidin-1-ylcarbonyl)-3-(4-methylphenylcarbonylamino)-phenyl]benzamide;
N-hydroxy-4-[3-(4-methylphenylcarbonylamino)-5-(pyrrolidin-1-ylcarbonyl)phenyl]-benzamide;
N-hydroxy-4-[5-(3-(RS)-hydroxymethylpiperidin-1-ylcarbonyl)-3-(4-methylphenylcarbonyl-amino)phenyl]benzamide;
N-hydroxy-4-[5-(2-(RS)-hydroxymethylpiperidin-1-ylcarbonyl)-3-(4-methylphenylcarbonyl-amino)phenyl]benzamide;
N-hydroxy-4-{5-[N-(2-hydroxyethyl)-N-methylaminocarbonyl]-3-(4-methylphenylcarbonyl-amino)phenyl}benzamide;
Nhydroxy-4-[5-(N-4-chlorobenzylaminocarbonyl)-3-(4-methylphenylcarbonylamino)-phenyl]benzamide;
N-hydroxy-4-[5-(N-3-methylbutylaminocarbonyl)-3-(4-methylphenylcarbonylamino)-phenyl]benzamide;
N-hydroxy-4-[3-(4-methylphenylcarbonylamino)-5-(N-2-phenylethylaminocarbonyl)phenyl]-benzamide;
N-hydroxy-4-{5-[N-2-(4-methoxyphenyl)ethylaminocarbonyl]-3-(4-methylphenylcarbonyl-amino)phenyl}benzamide;
N-hydroxy-4-[3-(2-methylphenylcarbonylamino)phenyl]benzamide;
N-hydroxy-4-[3-(2,4-dimethylphenylcarbonylamino)phenyl]benzamide;
N-hydroxy-4-[3-(2,5-dimethylphenylcarbonylamino)phenyl]benzamide;
N-hydroxy-4-[3-(2-methylthiophen-5-ylcarbonylamino)phenyl]benzamide;
N-hydroxy-4-[3-(4-methylphenylcarbonylamino)-5-(N-phenyl-N-methylaminocarbonyl)phenyl]-benzamide;
N-hydroxy-4-{3-[N-2-(4-methoxyphenyl)ethylaminocarbonyl]phenyl}benzamide;
N-hydroxy-4-[3-(Nchlorobenzylaminocarbonylmethyl)phenyl]benzamide;
N-hydroxy-4-[5-(N-benzylaminocarbonyl)-3-(4-methylphenylcarbonyl-amino)phenyl]benzamide;
N-hydroxy-4-{5-[N-(2-hydroxyethyl)aminocarbonyl]-3-(4-methylphenylcarbonyl-amino)phenyl}benzamide;
N-hydroxy-4-{5-[N,N-bis(2-hydroxyethyl)aminocarbonyl]-3-(4-methylphenylcarbonyl-amino)-phenyl]benzamide;
N-hydroxy-4-[3-(N,N-dimethylaminocarbonylmethyl)phenyl]benzamide;
N-hydroxy-4-[3-(morpholin-4-ylcarbonylmethyl)phenyl]benzamide;
N-hydroxy-4-[5-(4-(RS)-hydroxymethylpiperidin-1-ylcarbonyl)-3-(4-methylphenylcarbonyl-amino)phenyl]benzamide;
N-hydroxy-4-[5-(3-(RS)-ethoxycarbonylpiperidin-1-ylcarbonyl)-3-(4-methylphenylcarbonyl-amino)phenyl]benzamide;
N-hydroxy-4-[3-(N-phenyl-N-methylaminocarbonylmethyl)phenyl]benzamide;
N-hydroxy-4-[3-(N-2-phenylethylaminocarbonylmethyl)phenyl]benzamide;
N-hydroxy-4-[5-(4-(RS)-aminocarbonylpiperidin-1-ylcarbonyl)-3-(4-methylphenylcarbonyl-amino)phenyl]benzamide;
N-hydroxy-4-[5-(2-(RS)-aminocarbonylpyrrolidin-1-ylcarbonyl)-3-(4-methylphenylcarbonyl-amino)phenyl]benzamide;
N-hydroxy-4-[5-(4-(RS)-ethoxycarbonylpiperidin-1-ylcarbonyl)-3-(4-methylphenylcarbonyl-amino)phenyl]benzamide;
N-hydroxy-4-{5-[N-(N-methylaminocarbonylmethyl)aminocarbonyl]-3-(4-methylphenyl-carbonyl-amino)phenyl}benzamide;
N-hydroxy-4-{5-[N-(N-dimethylaminocarbonylmethyl)-N-methylaminocarbonyl]-3-(4-methylphenylcarbonyl-amino)phenyl}benzamide;
N-hydroxy-4-(5-[N-(aminocarbonylmethyl)-N-methylaminocarbonyl]-3-(4-methylphenyl-carbonyl-amino)phenyl}benzamide;
N-hydroxy-4-(5-[N-(2-hydroxy-1-hydroxymethyl)ethylaminocarbonyl]-3-(4-methylphenyl-carbonyl-amino)phenyl}benzamide;
N-hydroxy-4-[3-(2,4-dichlorophenylcarl)onylamino)-5-(pyrrolidin-1-ylcarbonyl)phenyl]-benzamide;
N-hydroxy-4-[3-(N-4-chlorophenylaminocarbonylmethyl)phenyl]benzamide;
N-hydroxy-4-[3-(2,4-dichlorophenylcarbonylamino)-5-(piperidin-1-ylcarbonyl)phenyl]-benzamide;
N-hydroxy-4-[3-(2,4-dichlorophenylcarbonylamino)-5-(2-(S)-hydroxymethylpyrrolidin-1-ylcarbonyl)phenyl]-benzamide;
N-hydroxy-4-[3-(2,4-dichlorophenylcarbonylamino)-5-(2-(R)-hydroxymethylpyrrolidin-1-ylcarbonyl)phenyl]-benzamide;
N-hydroxy-4-(4-benzyloxybenzimidazol-2-yl)benzamide;
N-hydroxy-4-[3-(4-methoxyphenylcarbonylamino)phenyl]benzamide;
N-hydroxy-4-[3-(4-N-benzylaminocarbonyl)phenyl]benzamide;
N-hydroxy-4-[3-(N-2,2,2-trifluoroethylaminocarbonyl)phenyl]benzamide;
N-hydroxy-4-[3-(2,4-dichlorophenylcarbonylamino)-5-(morpholin-4-ylcarbonyl)phenyl]-benzamide;
N-hydroxy-4-[3-(4-methylphenylcarbonylamino)-5-(N-methoxy-N-methylaminocarbonyt)-phenyl]-benzamide;
N-hydroxy-4-[3-(4-methylphenylcarbonylamino)-5-(4-chlorophenylaminocarbonyl)phenyl]-benzamide;
N-hydroxy-4-[3-(benzylcarbonylamino)-5-(pyrrolidin-1-ylcarbonyl)phenyl]-benzamide;
N-hydroxy-4-[3-(2-phenylethylcarbonylamino)-5-(pyrrolidin-1-ylcarbonyl)phenyl]-benzamide;
N-hydroxy-4-[3-(4-methoxy-2-methylphenylcarbonylamino)-5-(pyrrolidin-1-ylcarbonyl)-phenyl]-benzamide;
N-hydroxy-4-[3-(4-methoxy-2-methylphenylcarbonylamino)-5-(piperidin-1-ylcarbonyl)-phenyl]-benzamide;
N-hydroxy-4-[3-(2-phenylethenylenecarbonylamino)-5-(pyffolidin-1-ylcarbonyl)phenyl]-benzamide;
N-hydroxy-4-[3-(2,4-dimethylphenylcarbonylamino)-5-(pyrrolidin-1-ylcarbonyl)phenyl]-benzamide;
N-hydroxy-4-[3-(2,4-dimethylphenylcarbonylamino)-5-(piperidin-1-ylcarbonyl)phenyl]-benzamide;
N-hydroxy-4-[3-(2,4-dimethylphenylcarbonylamino)-5-(diethylaminocarbonyl)phenyl]-benzamide;
N-hydroxy-4-[3-(4-methoxy-2-methylphenylcarbonylamino)-5-(diethylaminocarbonyl)-phenyl]-benzamide;
N-hydroxy-4-[3-(2,4-dichlorophenylcarbonylamino)-5-(dimethylaminomethyl)phenyl]-benzamide;
N-hydroxy-4-[3-(4-methylphenylcarbonylamino)-5-(dimethylaminomethyl)phenyl]-benzamide;
N-hydroxy-4-[3-(phenylsulfonylamino)-5-(propoxymethyl)phenyl]-benzamide;
N-hydroxy-4-[3-(2,4-dichlorophenylcarbonylamino)-5-(piperidin-1-ylmethyl)phenyl]-benzamide;
N-hydroxy-4-[3-(4-methoxyphenylcarbonylamino)-5-(propoxymethyl)phenyl]-benzamide;
N-hydroxy-4-[3-(2-propylcarbonylamino)-5-(propoxymethyl)phenyl]-benzamide;
N-hydroxy-4-[3-(3-phenylureido)-5-(propoxymethyl)phenyl]-benzamide;
N-hydroxy-4-[3-(benzylamino)-5-(propoxymethyl)phenyl]-benzamide;
N-hydroxy-4-[3-(phenylethenylcarbonylamino)-5-(propoxymethyl)phenyl]-benzamide;
N-hydroxy-4-[3-(phenylaminomethylcarbonylamino)-5-(propoxymethyl)phenyl]-benzamide,
N-hydroxy-4-[3-(4-methoxyphenylcarbonylamino)-5-(benzyloxymethyl)phenyl]-benzamide;
N-hydroxy-4-[3-(4-methoxyphenylcarbonylamino)-5-(phenoxymethyl)phenyl]-benzamide;
N-hydroxy-4-[3-(4-methoxyphenylcarbonylamino)-5-(phenylethenylmethyloxymethyl)phenyl]-benzamide;
N-hydroxy-4-[3-(2,4-dichlorophenylcarbonylamino)-5-(pyrrolidin-1-ylmethyl)phenyl]-benzamide;
N-hydroxy-4-[3-(indol-3-ylmethylcarbonylamino)-5-(dimethylaminocarbonyl)phenyl]-benzamide;
N-hydroxy-4-[3-(2,4-dichlorobenzylamino)-5-(dimethylaminocarbonyl)phenyl]-benzamide;
N-hydroxy-4-[3-(pyridin-4-ylmethylcarbonylamino)-5-(dimethylaminocarbonyl)phenyl]-benzamide;
N-hydroxy-4-[3-(2,4-dichlorophenylcarbonylamino)-5-[N-(2-hydroxyethyl)-N-methylaminocarbonyl)phenyl]-benzamide;
N-hydroxy-4-[3-(furan-3-ylcarbonylamino)-5-(dimethylaminomethyl)phenyl]-benzamide;
N-hydroxy-4-[3-(3-phenylureido)phenyl]benzamide;
N-hydroxy-4-[3-(4-methoxyphenylcarbonylamino)phenyl]benzamide;
N-hydroxy-4-[3-(benzylamino)phenyl]benzamide;
N-hydroxy-4-[3-(4-methoxybenzylamino)phenyl]benzamide;
N-hydroxy-4-[3-(4-methylbenzylamino)phenyl]benzamide;
N-hydroxy-4-[3-(phenylcarbonylaminomethyl)phenyl]benzamide;
N-hydroxy-4-[3-(4-chlorophenylaminocarbonylmethyl)phenyl]benzamide;
N-hydroxy-4-[3-(pyridin-3-ylmethylcarbonylaminomethyl)phenyl]benzamide;
N-hydroxyA4-{3-[N-(benzoyl)-N-(2-hydroxyethyl)aminomethyl]phenyl}benzamide;
N-hydroxy-4-{3-[N-(benzyl)-N-(2-hydroxyethyl)aminomethyl]phenyl}benzamide;
N-hydroxy-4-{3-[N-(benzyl)-N-(2-methoxyethyl)aminomethyl]phenyl}benzamide;
N-hydroxy-4-[3-(benzylaminomethyl)phenyl}benzamide;
N-hydroxy-4-[3-(2-indol-3-ylethylaminomethyl)phenyl}benzamide;
N-hydroxy-4-[3-(3,4-methylenedioxybenzylaminomethyl)phenyl}benzamide;
N-hydroxy-4-[3-(pyridin-4-ylmethylaminomethyl)phenyl}benzamide;
N-hydroxy-4-[3-(pyridin-3-yloxymethyl)phenyl}benzamide;
N-hydroxy-4-[3-(2-pyridin-3-ylethylaminomethyl)phenyl}benzamide;
N-hydroxy-4-[3-(phenyloxymethyl)phenyl}benzamide;
N-hydroxy-4-[3-(4-methylphenyloxymethyl)phenyl}benzamide;
N-hydroxy-4-[3-(4-chlorophenyloxymethyl)phenyl}benzamide;
N-hydroxy-4-[3-(3-phenylpropylaminomethyl)phenyl}benzamide;
N-hydroxy-4-[3-(phenylaminomethyl)phenyl}benzamide;
N-hydroxy-4-[3-(4-methylphenylaminomethyl)phenyl}benzamide;
N-hydroxy-4-[3-(4-chlorophenylaminomethyl)phenyl lbenzamide;
N-hydroxy-[5-(morpholin-4-ylcarbonyl)-3-(4-methylphenylcarbonylamino)phenyl]-benzaride; or
N-hydroxy-4-[3-(pyridin-3-ylmethylcarbonylaminomethyl)phenyl]benzamide; or
a pharmaceutically acceptable salt thereof.

12. The compound of claim 11 wherein the compound is selected from the group consisting of:
N-hydroxy-4-(3-methylsulfonylaminophenyl)benzamide;
N-hydroxy-4-(3-phenylsulfonylaminophenyl)benzamide;
N-hydroxy-4-[3-(3-chlorophenylsulfonylamino)phenyl]benzamide;
N-hydroxy-4-[3-(4-fluorophenylsulfonylamino)phenyl]benzamide;
N-hydroxy-4-[3-(3,4-dichlorophenylsulfonylamino)phenyl]benzamide;
N-hydroxy-4-[3-(phenylcarbonylamino)phenyl]benzamide;
N-hydroxy-4-[3-(benzylsulfonylamino)phenyl]benzamide;
N-hydroxy-4-[3-(4-chlorophenylsulfonylamino)phenyl]benzamide;
N-hydroxy-4-[3-(3-trifluoromethylphenylsulfonylamino)phenyl]benzamide;
N-hydroxy-4-[3-(4-methoxyphenylsulfonylamino)phenyl]benzamide;
N-hydroxy-4-[3-(3,4-dichlorophenylcarbonylamino)phenyl]benzamide;
N-hydroxy-4-[3-(3-methoxyphenylureido)phenyl]benzamide;
N-hydroxy-4-[3-(3,4-dimethoxyphenylcarbonylamino)phenyl]benzamide;
N-hydroxy-4-[3-(2,5-dimethoxyphenylsulfonylamino)phenyl]benzamide;
N-hydroxy-4-[3-(4-trifluoromethoxyphenylsulfonylamino)phenyl]benzamide;
N-hydroxy-4-[3-(3-fluorophenylcarbonylamino)phenyl]benzamide;
N-hydroxy-4-[3-(2,4-dichlorophenylcarbonylamino)phenyl]benzamide;
N-hydroxy-4-[3-(phenylcarbonylamino)phenyl]benzamide;
N-hydroxy-4-[3-(4-methylphenylcarbonylamino)phenyl]benzamide;
N-hydroxy-4-[3-(3-trifluoromethylphenylcarbonylamino)phenyl]benzamide;
N-hydroxy-4-[3-(3,4-methylenedioxyphenylcarbonylamino)phenyl]benzamide;
N-hydroxy-4-[3-(2-propylcarbonylamino)phenyl]benzamide;
N-hydroxy-4-[3-(benzylcarbonylamino)phenyl]benzamide;
N-hydroxy-4-[3-(2-phenylethylcarbonylamino)phenyl]benzamide;
N-hydroxy-4 {3-[2-(4-trifluorophenyl)ethylcarbonylamino]phenyl}benzamide;
N-hydroxyA-{3-[2-(4-methoxyphenyl)ethylcarbonylamino]phenyl}benzamide;
N-hydroxy-4-[3-(3,5-dichlorophenylcarbonylamino)phenyl]benzamide;
N-hydroxy-4-[3-(phenoxymethylcarbonylamino)phenyl]benzamide;
N-hydroxy-4-[3-(3-methylbutylcarbonylamino)phenyl]benzamide;
N-hydroxy-4-[3-(thiophen-2-ylmethylcarbonylamino)phenyl]benzamide;
N-hydroxy-4-{3-[2-(2,4-dichlorophenyl)ethylcarbonylamino]phenyl}benzamide;
N-hydroxy-4-{3-[2-(3,4-methylenedioxyphenyl)ethylcarbonylamino]phenyl]benzamide;
N-hydroxy-4-[3-(4-trifluorophenylcarbonylamino)phenyl]benzamide;
N-hydroxy-4-[3-(4-ethoxyphenylcarbonylamino)phenyl]benzamide;
N-hydroxy-4-[3-(4-N,N-dimethylaminophenylcarbonylamino)phenyl]benzamide;
N-hydroxy-4-[3-(4-fluorophenylcarbonylamino)phenyl]benzamide;
N-hydroxy-4-[3-(2,4-difluorophenylcarbonylamino)phenyl]benzamide;
N-hydroxy-4-[3-(4-chlorophenylcarbonylamino)phenyl]benzamide;
N-hydroxy-4-[3-(N,N-dimethylaminocarbonyl)phenyl]benzamide;
N-hydroxy-4-[3-(4-isopropylphenylcarbonylamino)phenyl]benzamide;
N-hydroxy-4-[3-(4-trifluoroinethoxyphenylcarbonylamino)phenyl]benzamide;
N-hydroxy-4-[3-(3-fluoro-4-methoxyphenylcarbonylamino)phenyl]benzamide;
N-hydroxy-4-[3-(4-methoxy-2-methylphenylcarbonylamino)phenyl]benzamide;
N-hydroxy-4-[3-(2,4-dimethoxyphenylcarbonylamino)phenyl]benzamide;
N-hydroxy-4-[3-(4-chloro-2-methoxyphenylcarbonylamino)phenyl]benzamide;
N-hydroxy-4-[3-(pyridin-4-ylcarbonylamino)phenyl]benzamide;
N-hydroxy-4-[3-(pyridin-3-ylcarbonylamino)phenyl]benzamide;
N-hydroxy-4-[3-(2-methylthiophen-5-ylcarbonylamino)phenyl]benzamide;
N-hydroxy-4-[3-(N-4-chlorobenzylaminocarbonylmethyl)phenyl]benzamide;
N-hydroxy-4-[3-(N,N-dimethylaminocarbonylmethyl)phenyl]benzamide;
N-hydroxy-4-[3-(morpholin-4-ylcarbonylmethyl)phenyl]benzamide;
N-hydroxy-4-[3-(N-phenyl-N-methylaminocarbonylmethyl)phenyl]benzamide;
N-hydroxy-4-[3-(N-2-phenylethylaminocarbonylmethyl)phenyl]benzamide;
N-hydroxy-4-[3-(3-phenylureido)phenyl]benzamide;
N-hydroxy-4-[3-(4-methoxyphenylcarbonylamino)phenyl]benzamide;
N-hydroxy-4-[3-(benzylamino)phenyl]benzamide;
N-hydroxy-4-[3-(4-methoxybenzylamino)phenyl]benzamide;
N-hydroxy-4-[3-(4-methylbenzylamino)phenyl]benzamide;
N-hydroxy-4-[3-(phenylcarbonylaminomethyl)phenyl]benzamide;
N-hydroxy-4-[3-(4-chlorophenylaminocarbonyhmethyl)phenyl]benzamide;
N-hydroxy-4-[3-(benzylcarbonylaminomethyl)phenyl]benzamide;
N-hydroxy-4-{3-[N-(benzoyl)-N-(2-hydroxyethyl)aminomethyl]phenyl}benzamide;
N-hydroxy-4-{3-[N-(benzyl)-N-(2-hydroxyethyl)aminomethyl]phenyl}benzamide;
N-hydroxy-4-{3-[N-(benzyl)-N-(2-methoxyethyl)aminomethyl]phenyl}benzamide;
N-hydroxy-4-[3-(benzylaminomethyl)phenyl}benzamide;
N-hydroxy-4-[3-(2-indol-3-ylethylaminomethyl)phenyl}benzamide;
N-hydroxy-4-[3-(3,4-methylenedioxybenzylaminomethyl)phenyl}benzamide;
N-hydroxy-4-[3-(pyridin-4-ylmethylaminomethyl)phenyl}benzamide;
N-hydroxy-4-[3-(pyridin-3-yloxymethyl)phenyl}benzamide;
N-hydroxy-4-[3-(2-pyridin-3-ylethylaminomethyl)phenyl}benzamide;
N-hydroxy-4-[3-(phenyloxymethyl)phenyl}benzamide;
N-hydroxy-4-[3-(4-methylphenyloxymethyl)phenyl}benzamide;
N-hydroxy-4-[3-(4-chlorophenyloxymethyl)phenyl}benzamide;
N-hydroxy-4-[3-(3-phenylpropylaminomethyl)phenyl}benzamide;
N-hydroxy-4-[3-(phenylarainomethyl)phenyl}benzamide;
N-hydroxy-4-[3-(4-methylphenylaminomethyl)phenyl}benzamide;
N-hydroxy-4-[3-(4-chlorophenylaminomethyl)phenyl}benzamide;
N-hydroxy-4-[3-(2-phenylethylaminocarbonylmethyl)phenyl]benzamide;
N-hydroxy-4-[3-(pyridin-3-ylmethylcarbonylaminomethyl)phenyl]benzamide;
N-hydroxy-4-[3-(2-methylphenylcarbonylamino)phenyl]benzamide;
N-hydroxy-4-[3-(2,4-dimethylphenylcarbonylamino)phenyl]benzamide;
N-hydroxy-4-[3-(2,5-dimethylphenylcarbonylamino)phenyl]benzaniide; and
N-hydroxy-4-[3-(4-methoxyphenylcarbonylamino)phenyl]benzamide; or
a pharmaceutically acceptable salt thereof.

13. The compound of claim 11 wherein the compound is selected from the group consisting of:
N-hydroxy-4-[5-(phenylaminocarbonyl)-3-(phenylcarbonylamino)phenyl]benzamide;
N-hydroxy-4-[5-(phenylaminocarbonyl)-3-(phenylsulfonylamino)phenyl]benzamide;
N-hydroxy-4-[5-(N-benzylaminocarbonyl)-3-(phenylcarbonylamino)phenyl]-benzamide;
N-hydroxy-4-[5-carboxy-3-(phenylcarbonylamino)phenyl]benzamide;
N-hydroxy-4-[5-(N,N-dimethylaminocarbonyl)-3-(phenylcarbonylamino)phenyl]-benzamide;
N-hydroxy-4-[5-(morpholin-4-ylcarbonyl)-3-(phenylcarbonylamino)phenyl]-benzamide;
N-hydroxy-4-[5-(N,N-dimethylaminocarbonyl)-3-(4-methylphenylcarbonylamino)-phenyl]-benzamide;
N-hydroxy-4-[3-(4-methylphenylcarbonylamino)-5-(N-phenylaminocarbonyl)-phenyl]-benzamide;
N-hydroxy-4-[5-(N,N-dimethylaminocarbonyl)-3-(4-methoxyphenylcarbonylamino)-phenyl]-benzamide;
N-hydroxy-4-[3-(4-methoxyphenylcarbonylamino)-5-(morpholin-4-ylcarbonyl)-phenyl]-benzamide;
N-hydroxy-4-[3-(4-methoxyphenylcarbonylamino)-5-(phenylaminocarbonyl)phenyl]-benzamide;
N-hydroxy-4-[5-(N,N-dimethylaminocarbonyl)-3-(3,4-dimethoxyphenyl-carbonylamino)-phenyl]-benzamide;
N-hydroxy-4-[3-(3,4-dimethoxyphenylcarbonylamino)-5-(morpholin-4-ylcarbonyl)-phenyl]-benzamide;
N-hydroxy-4-[3-(3,4-dimethoxyphenylcarbonylamino)-5-(phenylaminocarbonyl)-phenyl]-benzamide;
N-hydroxy-4-[3-(4-methylphenylcarbonylamino)-5-(piperidin-1-ylcarbonyl)phenyl]-benzamide;
N-hydroxy-4-[3-(4-methylphenylcarbonylamino)-5-(piperazin-1-ylcarbonyl)phenyl]-benzamide;
N-hydroxy-4-[5-(N-methylaminocarbonyl)-3-(4-methylphenylcarbonylamino)-phenyl]-benzamide;
N-hydroxy-4-[5-(N-carboxymethyl-N-methylaminocarbonyl)-3-(4-methylphenyl-carbonylamino)phenyl]benzamide;
N-hydroxy-4-[5-(N-aminocarbonylmethylaminocarbonyt)-3-(4-methylphenyl-carbonyl-amino)phenyl]-benzamide;
N-hydroxy-4-{5-[N-(2-N-methylaminocarbonylethyl)-N-(methyl)aminocarbonyl]-3-(4-methylphenylcarbonylamino)phenyl}-benzamide;
N-hydroxy-4-[5-(N-carboxymethylaminocarbonyl)-3-(4-methylphenylcarbonyl-amino)-phenyl]-benzamide;
N-hydroxy-4-[5-(N,N-dimethylaminocarbonyl)-3-(3,4-methylenedioxyphenyl-carbonyl-amino)phenyl]benzamide;
N-hydroxy-4-[3-(3,4-methylenedioxyphenylcarbonylamino)-5-(morpholin-4-ylcarbonyl)-phenyl]benzamide;
N-hydroxy-4-[3-(3,4-methylenedioxyphenylcarbonylamino)-5-(N-phenylamino-carbonyl)-phenyl]benzamide;
N-hydroxy-4-[3-(2,4-dichlorophenylcarbonylamino)-5-(N,N-dimethylamino-carbonyl)-phenyl]benzamide;
N-hydroxy-4-[3-(2,4-dichlorophenylcarbonylamino)-5-(N-phenylaminocarbonyl)-phenyl]-benzamide;
N-hydroxy-4-[5-(N₉N-dimethylaminocarbonyl)-3-(4-chlorophenylcarbonylamino)-phenyl]benzamide;
N-hydroxy-4-[5-(morpholin-4-ylcarbonyl)-3-(4-chlorophenylcarbonylamino)-phenyl]benzamide;
N-hydroxy-4-[5-(N-methyl-N-ethylaminocarbonyl)-3-(4-methylphenylcarbonyl-amino)-phenyl]benzamide;
N-hydroxy-4-[3-(4-methylphenylcarbonylamino)-5-(4-methylpiperazin-1-yl-carbonyl)-phenyl]-benzamide;
N-hydroxy-4-[5-(3-(RS)-aminocarbonylpiperidin-1-ylcarbonyl)-3-(4-methylphenyl-carbonyl-amino) phenyl]benzamide;
N-hydroxy-4-[5-(N,N-diethylaminocarbonyl)-3-(4-methylphenylcarbonylamino)-phenyl]benzamide;
N-hydroxy-4-{5-[N-2-(N-methylamino)ethylaminocarbonyl]-3-(4-methylphenyl-carbonylamino)-phenyl}benzamide;
N-hydroxy-4-[5-(4-hydroxypiperidin-1-ylcarbonyl)-3-(4-methylphenylcarbonyl-amino)-phenyl]benzamide;
N-hydroxy-4-[3-(4-methylphenylcarbonylamino)-5-(pyrrolidin-1-ylcarbonyl)phenyl]-benzamide;
N-hydroxy-4-[5-(3-(RS)-hydroxymethylpiperidin-1-ylcarbonyl)-3-(4-methylphenyl-carbonyl-amino)phenyl]benzamide;
N-hydroxy-4-[5-(2-(RS)-hydroxymethylpiperidin-1-ylcarbonyl)-3-(4-methylphenyl-carbonyl-amino)phenyl]benzamide;
N-hydroxy-4-{5-[N-(2-hydroxyethyl)-N-methylaminocarbonyl]-3-(4-methylphenyl-carbonyl-amino) phenyl}benzamide;
N-hydroxy-4-[5-(N-4-chlorobenzylaminocarbonyl)-3-(4-methylphenylcarbonyl-amino)-phenyl]benzamide;
N-hydroxy-4-[5-(N-3-methylbutylaminocarbonyl)-3-(4-methylphenylcarbonyl-amino)-phenyl]benzamide;
N-hydioxy-4-[3-(4-methylphenylcarbonylamino)-5-(N-2-phenylethylaminocarbonyl)-phenyl]-benzamide;
N-hydroxy-4-{5-[N-2-(4-methoxyphenyl)ethylaminocarbonyl]-3-(4-methylphenyl-carbonyl-amino)phenyl}benzamide;
N-hydroxy-4-[3-(4-methylphenylcarbonylamino)-5-(N-phenyl-N-methylamino-carbonyl)phenyl]-benzamide;
N-hydroxy-4-[5-(N-benzylaminocarbonyl)-3-(4-methylphenylcarbonyl-amino)-phenyl]benzamide;
N-hydroxy-4-{5-[N-(2-hydroxyethyl)aminocarbonyl]-3-(4-methylphenylcarbonyl-amino)phenyl}benzamide;
N-hydroxy-4-{5-[N,N-bis(2-hydroxyethyl)aminocarbonyl]-3-(4-methylphenyl-carbonyl-amino)-phenyl]benzamide;
N-hydroxy-4-[5-(4-(RS)-hydroxymethylpiperidin-1-ylcarbonyl)-3-(4-methylphenyl-carbonyl-amino)phenyl]benzamide;
N-hydroxy-4-[5-(3-(RS)-ethoxycarbonylpiperidin-1-ylcarbonyl)-3-(4-methylphenyl-carbonyl-amino)phenyl]benzamide;
N-hydroxy-4-[5-(4-(RS)-aminocarbonylpiperidin-1-ylcarbonyl)-3-(4-methylphenyl-carbonyl-amino)phenyl]benzamide;
N-hydroxy-4-[5-(2-(RS)-aminocarbonylpyrrolidin-1-ylcarbonyl)-3-(4-methylphenyl-carbonyl-amino)phenyl]benzamide;
N-hydroxy-4-[5-(4-(RS)-ethoxycarbonylpiperidin-1-ylcarbonyl)-3-(4-methylphenyl-carbonyl-amino)phenyl]benzamide;
N-hydroxy-4-{5-[N-(N-methylaminocarbonylmethyl)aminocarbonyl]-3-(4-methylphenyl-carbonyl-amino)phenyl}benzamide;
N-hydroxy-4-[3-(benzylcarbonylamino)-5-(pyrrolidin-1-ylcarbonyl)phenyl]-benzamide;
N-hydroxy-4-[3-(2-phenylethylcarbonylamino)-5-(pyrrolidin-1-ylcarbonyl)phenyl]-benzamide;
N-hydroxy-4-[3-(4-methoxy-2-methylphenylcarbonylamino)-5-(pyrrolidin-1-yl-carbonyl)-phenyl]-benzamide;
N-hydroxy-4-[3-(4-methoxy-2-methylphenylcarbonylamino)-5-(piperidin-1-yl-carbonyl)-phenyl]-benzamide;
N-hydroxy-4-[3-(2-phenylethenylenecarbonylamino)-5-(pyrrolidin-1-ylcarbonyl)-phenyl]-benzamide;
N-hydroxy-4-[3-(2,4-dimethylphenylcarbonylamino)-5-(pyrrolidin-1-ylcarbonyl)-phenyl]-benzamide;
N-hydroxy-4-[3-(2,4-dimethylphenylcarbonylamino)-5-(piperidin-1-ylcarbonyl)-phenyl]-benzamide;
N-hydroxy-4-[3-(2,4-dimethylphenylcarbonylamino)-5-(diethytaminocarbonyl)phenyl]-benzamide;
N-hydroxy-4-[3-(4-methoxy-2-methylphenylcarbonylamino)-5-(diethylamino-carbonyl)-phenyl]-benzamide;
N-hydroxy-4-[3-(2,4-dichlorophenylcarbonylamino)-5-(dimethylaminomethyl)phenyl]-benzamide;
N-hydroxy-4-[3-(4-methylphenylcarbonylamino)-5-(dimethylaminomethyl)phenyl]-benzamide;
N-hydroxy-4-[3-(phenylsulfonylamino)-5-(propoxymethyl)phenyl]-benzamide;
N-hydroxy-4-[3-(2,4-dichlorophenylcarbonylamino)-5-(piperidin-1-ylmethyl)phenyl]-benzamide;
N-hydroxy-4-[3-(4-methoxyphenylcarbonylamino)-5-(propoxymethyl)phenyl]-benzamide;
N-hydroxy-4-[3-(2-propylcarbonylamino)-5-(propoxymethyl)phenyl]-benzamide;
N-hydroxy-4-[3-(3-phenylureido)-5-(propoxymethyl)phenyl]-benzamide;
N-hydroxy-4-[3-(benzylamino)-5-(propoxymethyl)phenyl]-benzamide;
N-hydroxy-4-[3-(phenylethenylcarbonylamino)-5-(propoxymethyl)phenyl]-benzamide;
N-hydroxy-4-[3-(phenylaminomethylcarbonylamino)-5-(propoxymethyl)phenyl]-benzamide;
N-hydroxy-4-[3-(4-methoxyphenylarbonylamino)-5-(benzyloxymethyl)phenyl]-benzamide;
N-hydroxy-4-[3-(4-methoxyphenylcarbonylamino)-5-(phenoxymethyl)phenyl]-benzamide;
N-hydroxy-4-[3-(4-methoxyphenylcarbonylamino)-5-(phenylethenylmethyloxy-methyl)-phenyl]-benzamide;
N-hydroxy-4-[3-(2,4-dichlorophenylcarbonylamino)-5-(pyrrolidin-1-ylmethyl)phenyl]-benzamide;
N-hydroxy-4-[3-(indol-3-ylmethylcarbonylamino)-5-(dimethylaminocarbonyl)phenyl]-benzamide;
N-hydroxy-4-[3-(2,4-dichlorobenzytamino)-5-(dimethylaminocarbonyl)phenyl]-benzamide;
N-hydroxy-4-[3-(pyridin-4-ylmethylcarbonylamino)-5-(dimethylaminocarbonyl)-phenyl]-benzamide;
N-hydroxy-4-[3-(2,4-dichlorophenylcarbonylamino)-5-[N-(2-hydroxyethyl)-N-methylaminocarbonyl)phenyl]-benzamide;
N-hydroxy-4-[3-(furan-3-ylcarbonylamino)-5-(dimethylaminomethyl)phenyl]-benzamide;
N-hydroxy-[5-(morpholin-4-ylcarbonyl)-3-(4-methylphenylcarbonylamino)phenyl]-benzamide;
N-hydroxy-[5-(morpholin-4-ylcarbonyl)-3-(2,4-dichlorophenylcarbonylamino)-phenyl]-benzamide;
N-hydroxy-[5-(N-methoxy-N-methylaminocarbonyl)-3-(4-methylphenylcarbonyl-amino)-phenyl]benzamide; and
N-hydroxy-[5-(4-chlorophenylaminocarbonyl)-3-(4-methylphenylcarbonylamino)-phenyl]benzamide; or
a pharmaceutically acceptable salt thereof.

14. A pharmaceutical composition comprising a therapeutically effective amount of a compound of claim 1 or 2 or pharmaceutically acceptable salts thereof and a pharmaceutically acceptable excipient.

15. A method for treating a disease in an animal mediated by HADC which method comprises administering to the animal a pharmaceutical composition comprising a therapeutically effective amount of a compound of Formula I: 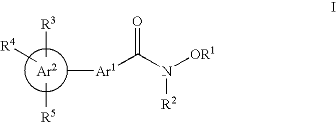
wherein:
R¹ is hydrogen or alkyl;
R² is hydrogen;
Ar¹ is phenylene or a six membered heteroarylene ring containing one or two nitrogen ring atoms, the rest of the ring atoms being carbon; wherein said Ar¹ group is optionally substituted with one or two groups independently selected from alkyl, halo, hydroxy, alkoxy, trifluoromethoxy, or trifluoromethyl;
Ar² is aryl, heteroaryl, cycloalkyl or heterocycloalkyl;
R³ is hydrogen, alkyl, halo, hydroxy, or alkoxy; and
R⁴ and R⁵ are independently selected from the group consisting of hydrogen, alkyl, halo, haloalkyl, nitro, cyano, carboxy, carboxyalkyl, alkoxycarbonyl, optionally substituted phenyl, optionally substituted heteroaryl, optionally substituted heterocycloalkyl, cycloalkyl, heterocycloaminoalkyl, —X—R⁶, or —(C₁₋₆alkylene)-Y—R⁷ where X and Y are independently —O—, —S—, —SO—, —SO₂—, —NR⁸—, —CO—, —NR⁹CO—, —CONR¹⁰—, —NR¹¹SO₂—, —SO₂NR¹²—, —NHC(O)O—, —OC(O)NH—, —NR¹³CONR¹⁴—, or —NR¹⁵SO₂NR¹⁶— where R⁶ and R⁷ are independently hydrogen, alkyl, hydroxyalkyl, optionally substituted phenyl, optionally substituted heteroaryl, optionally substituted heterocycloalkyl, cycloalkyl, optionally substituted phenylalkyl, optionally substituted phenoxyalkyl, optionally substituted phenylaminoalkyl, optionally substituted heteroaralkyl, optionally substituted heteroaryloxyalkyl, optionally substituted heterocycloalkylalkyl, or cycloalkylalkyl, R⁸, R⁹, R¹¹, R¹³, and R¹⁵ are independently hydrogen, alkyl, hydroxyalkyl, alkoxyalkyl, or optionally substituted phenylalkyl; R¹⁰, R¹², R¹⁴, and R¹⁶ are independently hydrogen, alkyl, optionally substituted phenylalkyl, alkoxy, hydroxyalkyl, haloalkyl, alkoxyalkyl, carboxyalkyl, cyanoalkyl, aminoalkyl, aminocarbonylalkyl, alkylaminocarbonylalkyl, dialkylaminocarbonylalkyl, or acyl or R⁴ and R⁵ together form methylenedioxy; and individual isomers, mixtures of isomers; or a pharmaceutically acceptable salt thereof and a pharmaceutically acceptable excipient.

16. The method of claim 15 wherein the disease is cancer and the animal is a human.

17. A method for treating cancer which method comprises administering to the animal a pharmaceutical composition comprising a therapeutically effective amount of a compound of claim 1 or 2 and a pharmaceutically acceptable excipient in combination with one or more compound(s) independently selected from an estrogen receptor modulator, an androgen receptor modulator, retinoid receptor modulator, a cytotoxic agent, another antiproliferative agent, a prenyl-protein transferase inhibitor, an HMG-CoA reductase inhibitor, an HIV protease inhibitor, a reverse transcriptase inhibitor, or an angiogenesis inhibitor.

18. A method for treating cancer which method comprises administering to the animal a pharmaceutical composition comprising a therapeutically effective amount of a compound of claim 1 or 2 and a pharmaceutically acceptable excipient in combination with radiation therapy and one or more compound(s) independently selected from an estrogen receptor modulator, an androgen receptor modulator, retinoid receptor modulator, a cytotoxic agent, another antiproliferative agent, a prenyl-protein transferase inhibitor, an HMG-CoA reductase inhibitor, an HIV protease inhibitor, a reverse transcriptase inhibitor, or an angiogenesis inhibitor.
